(19) United States
(12) Patent Application Publication
(10) Pub. No.: US 2024/0374827 A1
(43) Pub. Date: Nov. 14, 2024

(54) AUTO INJECTOR WITH MEASUREMENT OF BATTERY CAPACITY LEFT BEFORE RE-CHARGING IS REQUIRED

(71) Applicant: Ascendis Pharma A/S, Hellerup (DK)

(72) Inventors: Henrik Egesborg, Hellerup (DK); Abel Arturo Arredondo Zamudio, Struer (DK); Kurt Stæcker Jensen, Struer (DK); John Nørskov Christensen, Struer (DK)

(21) Appl. No.: 18/684,567

(22) PCT Filed: Sep. 29, 2022
(86) PCT No.: PCT/EP2022/077091

§ 371 Date: Feb. 16, 2024

(30) Foreign Application Priority Data
Sep. 30, 2021 (EP) 21200271.1

(51) Int. Cl. A61M 5/24; G01R 31/3835

(52) U.S. Cl. CPC A61M 5/2422; G01R 31/3835; A61M 2205/3368; A61M 2205/587; A61M 2205/6018; A61M 2205/8206

(57) ABSTRACT

Described herein is an auto injector for administering a medicament, the auto injector comprising a function, which initiate the auto injector process only if the auto injector battery is sufficiently charged.

The present disclosure relates to an auto injector, such as an electronic auto injector, and a system comprising an auto injector and a cartridge. The auto injector comprising a function, which allows a user to initiate the auto injector process only if the auto injector battery is sufficiently charged.

### BACKGROUND

Auto injectors, such as electronic auto injectors, have been developed and are widely used to aid the administering of fluid or medicaments to the body. The most common type of auto-injection devices adapted to receive a drug filled cartridge (also termed reservoir or container) and expel a dose therefrom are generally elongated e.g. pen-formed for being hold in a user's one hand and utilizes a so-called cartridge holder adapted to receive and mount a cartridge in the device. Correspondingly, most pen-formed drug delivery devices comprise a generally cylindrical cartridge holder for receiving and holding a generally cylindrical drug-filled cartridge in a mounted position, the cartridge comprising a proximally facing and axially displaceable piston, and a main body with a housing in which a drug expelling mechanism is arranged, the mechanism comprising an axially displaceable piston rod adapted to engage the piston of a mounted cartridge to thereby expel a dose of drug from the cartridge. Between the cartridge holder and the main body coupling means are provided allowing a user to remove the cartridge holder from the main body and reattach it when a used cartridge has been exchanged with a new cartridge. The cartridge is inserted in the cartridge holder by axial movement through a proximal opening. Conventionally, the coupling means are in the form of a threaded connection or a bayonet coupling.

However, utilizing electronic means causes a risk of the electric current passing through the body, thereby causing electric shock in the user. Especially since conventional hypodermic needles are made of metal and thus electrically conductive. Electric shock may cause serious injury, which may be potentially life threatening, especially if the device is coupled to the main power grid.

Safety is an important issue, especially in the field of medical devices, such as auto injectors. Therefore, precautionary measures are needed to prevent or reduce the risk of causing electrical shock in users of an auto injector. Furthermore, precautionary measures are subject of industry standards, such as ISO 11608 and IEC 60601 relating to needle-based injection systems for medical use and medical electrical equipment.

WO2017114912 describes an auto injector with a charger safety function, which ensures that the opening in the auto injector housing allowing for charging of the battery inside the auto injector cannot be access when a cartridge is inserted into the auto injector.

Some auto injectors further include features, which ensure that a cartridge remains locked inside the auto injector once an auto injector process has been initiated. The cartridge may be locked by movement of a plunger rod into the cartridge, e.g. for expelling of medicament. An example of such locking solution is described in WO2017114906.

If combining the cartridge locking of WO2017114906 and the charger safety function of WO2017114912, a situation may therefore occur where there will not be enough battery power to drive the plunger rod to performed medicament delivery and afterwards return to a position, where the cartridge is unlocked from the auto injector, thereby allowing the cartridge to be removed from the auto injector after use, if the auto injector battery runs out of power midway through an medicament delivery process. If the auto injector runs out of power, it is rendered unusable forever, because the cartridge cannot be released from the auto injector without battery power and for safety reasons, the device cannot be charged when a cartridge is inserted.

US20180236181A1 discloses an auto injector comprising a main control unit, which determines whether a battery has sufficient charge to complete a full drug delivery process including a warming process of the drug prior the initiation of the drug delivery process. If the battery has enough charge, the auto injector device may prompt the user to initiate the drug delivery process. Alternatively, if the battery does not have enough charge, the auto injector device may display a request message to charge the battery prior to initiation of the drug delivery process. The auto injector needs more energy at lower temperatures to perform a full drug delivery process due to the increased viscosity of the drug solution. Therefore, the auto injector comprises a heating unit. If the auto injector determines that the temperature is below a predefined threshold, the auto injector automatically operates the heating unit to warm up the drug solution to a suitable operating temperature prior drug administration.

There is therefore a need for an auto injector, where it is ensured that the battery does not run out of power before the cartridge may be removed from the auto injector allowing the auto injector battery to be recharged. There is also a need for a more compactly built auto injector with a reduced number of functional parts, e.g. a heating unit and that ensures that the auto injector does not run out of power during a drug delivery process, even at lower temperatures.

### SUMMARY

In auto injectors with re-chargeable batteries and a user interface with a module instructing the user to re-charge the battery when the remaining amount of battery voltage is at or lower than predetermined minimum battery voltage value, the minimum battery voltage value will often be provided at room temperature. This is, however, problematic when using the auto injector for delivery of a highly viscous medicament, as the lower the temperature of the medicament is, the higher the viscosity of the liquid medicament becomes. This effect is seen more pronounced when the temperature is below 15 degrees Celsius for many highly viscus liquids. Since the force needed to expel a liquid medicament through a small cartridge outlet increases as the viscosity of the medicament increases, more battery voltage is needed to expel a highly viscous medicament through a thin needle than to expel a liquid of low viscosity. A situation may therefore occur where the voltage drops to zero and the battery runs out of power before the auto injector process, e.g. a medicament expelling process, is completed if the temperature of the medicament is lower than the temperature, which has been used for defining the lower predetermined value.

Further, the performance of the battery depends on the temperature. Thus, the lower the temperature of the battery is, the faster the battery runs out of power and need a recharging.

Disclosed herein is therefore an auto injector for administering a medicament, the auto injector comprising:
a cartridge receiver configured to receive a cartridge, the cartridge comprising a cartridge outlet, a cartridge compartment containing the medicament, and a first stopper;
a plunger rod configured to move the first stopper inside the cartridge compartment for expelling the medicament through the cartridge outlet;
a drive module configured to move the plunger rod from a retracted plunger rod position to an extended plunger rod position;
a temperature sensor configured to measure a temperature of the auto injector;
a re-chargeable battery configured to power at least the drive module when moving the plunger rod;
a battery calculation module configured to calculate a residual electrical battery voltage level of the re-chargeable battery;
a processing unit coupled to the temperature sensor, the battery calculation module, and the drive module.

The residual electrical battery voltage level of the re-chargeable battery may provide a measure of the remaining residual electrical battery power.

The processing unit may be configured to:
receive the measured temperature from the temperature sensor;
receive the calculated residual electrical battery voltage level of the re-chargeable battery from the battery calculation module;
obtain a predefined threshold value indicative of the minimum electrical battery voltage level needed for performing an auto injector process at the temperature measured by the temperature sensor;
compare the calculated residual electrical battery voltage level with the obtained predefined threshold value.

The processing unit may further be configured to initiate the auto injector process only if the calculated residual electrical battery voltage level is larger than the predefined threshold value.

Disclosed herein is also a method for ensuring that a re-chargeable battery in an auto injector contains sufficient voltage to allow for the auto injector to perform an auto injector process, wherein the auto injector comprises:
a cartridge receiver configured to receive a cartridge containing medicament;
a temperature sensor configured to measure a temperature of the auto injector;
a re-chargeable battery configured to power at least a drive module for moving a plunger rod in the auto injector;
a battery calculation module configured to calculate a residual electrical battery voltage level of the re-chargeable battery;
a processing unit coupled to the temperature sensor, the battery calculation module, and the drive module, the processing unit being configured to perform the method.

The method comprises:
receiving the measured temperature from the temperature sensor;
receiving the calculated residual electrical battery voltage level of the re-chargeable battery from the battery calculation module;
obtaining a predefined threshold value indicative of the minimum electrical battery voltage level needed for performing the auto injector process at the measured temperature;
comparing the calculated residual electrical battery voltage level with the obtained predefined threshold value.

The method may further comprise initiating the auto injector process only if the calculated residual electrical battery voltage level is larger than the predefined threshold value.

Disclosed herein is additionally a system comprising an auto injector and a cartridge comprising a cartridge outlet, a cartridge compartment containing the medicament, and a first stopper.

By predefined threshold value is meant a measure of the minimum electrical battery voltage needed to perform an auto injector process. The predefined threshold value may also be obtained indirectly by calculating an estimated count of the remaining injection cycles, which a user is able to perform without a recharging of the battery.

Through the introducing of a check for the remaining battery voltage level at the time of activating the auto injector, the processing unit will be able to deny activating the auto injector process of advancing the plunger rod, which may lock the cartridge inside the auto injector, in case the battery voltage level is below an acceptable limit. This ensures that the cartridge is not stuck in the auto injector without an option of recharging the battery to allow for movement of the plunger rod to a position where the cartridge is unlocked and removable from the auto injector again. The auto injector according to the above, accounts for the temperature effect on the viscosity of the medicament and for the temperature dependent battery performance. The measurement of the temperature sensor ensures that the medicament viscosity correlated to the battery voltage needed to reconstitute and injection the medicament, can be taken into account when calculating the lowest acceptable battery voltage level needed to carry out the auto injector process.

By the above auto injector, system and method are advantageously obtained an auto injector, where it is ensured that the battery is sufficiently charged before an auto injector process is allowed to be initiated. Thus, it may be ensured that a full auto injector process may be completed before an additional re-charging of the battery is required independently of at which temperature the auto injector is used for performing the medicament delivery process.

Thus, it is further ensured that a cartridge is not stuck in the auto injector mid-way during an auto injector process, such as a medicament delivery process, as a consequence of the battery running out of power during the process.

It is envisaged that any embodiments or elements as described in connection with any one aspect may be used with any other aspects or embodiments, mutatis mutandis.

By temperature of the auto injector is meant a temperature measured by the auto injector temperature sensor anywhere inside the auto injector. In one or more examples, the temperature of the auto injector is one or more of:
an ambient temperature; and/or
a temperature near the medicament in the cartridge; and/or
a temperature indicative of a temperature of the medicament in the cartridge; and/or
a temperature of the auto injector near the battery; and/or
a temperature indicative of the temperature of the battery; and/or
any combination of the above.

Most often, the temperature of the auto injector will be the ambient temperature, possibly measured near the battery inside the auto injector. The ambient temperature may also characterize the battery's capability to drive the auto injector system, such that at low temperatures, the battery may be limited in supporting the auto injector process.

In one or more examples, the lowest acceptable battery voltage level is programmed into a control system in the auto injector. The lowest acceptable battery voltage level may therefore be obtained/calculated by the control system after obtaining the measured temperature. The lowest acceptable battery voltage level may be higher for lower temperatures.

In one or more examples, the processing unit may further be configured to instruct the user to recharge the battery if the calculated residual electrical battery voltage level is smaller than the predefined threshold value needed to perform the auto injector process at the measured temperature.

In one or more examples, the temperature sensor, the battery calculation module, and the processing unit are configured to perform the steps outlined above and to instruct the user to recharge the battery if the calculated residual electrical battery voltage level is smaller than the predefined threshold value needed to perform the auto injector process at the measured temperature without a cartridge being received in the auto injector. Thus, the auto injector performs a test of the battery capacity at the measured temperature and indicates to the user that the battery needs to be recharged before initiation of a medicament delivery auto injector process may occur. Alternatively, the cartridge may be inserted into the auto injector while the processing unit checks whether there is enough electrical battery voltage level to perform the auto injector process at the measured temperature. However, if there is not enough electrical battery voltage level to perform the auto injector process, the plunger rod will not be allowed to move from the retracted position to initiate the auto injector process.

The auto injector process may be one or more of:
a first plunger rod movement process, where the plunger rod is moved from the retracted plunger rod position to a locking plunger rod position, where a cartridge is locked inside the auto injector;
a second plunger rod movement process, where the plunger rod is moved from the locking plunger rod position to a first stopper plunger rod position bringing the plunger rod in contact with the first stopper;
a third plunger rod movement process, where the plunger rod moves the first stopper to cause a second stopper inside the cartridge to move to a bypass position allowing for mixing of medicament components positioned in separate sub-compartments upon delivery of the cartridge;
a fourth plunger rod movement process, where the plunger rod moves the first stopper to contact the second stopper for mixing of medicament components the first cartridge sub-compartment and the second cartridge sub-compartment;
a fourth plunger rod movement process, where the plunger rod is moved to the extended plunger rod position, where at the extended plunger rod position, medicament has been expelled from the cartridge, such as fully expelled from the cartridge;
a re-setting of the auto injector to an original position where the cartridge can be removed from the auto injector;
a full medicament delivery process comprising a combination of the above processes.

The fourth plunger rod movement process may also be referred to as a medicament reconstitution process.

The fifth plunger rod movement process may also be referred to as a medicament expelling process.

During the re-setting of the auto injector, the plunger rod moves to the retracted position.

When the cartridge is removed from the auto injector, inserting a new cartridge in the auto injector is possible.

By reconstitution process is meant a process of mixing a dry medicament component with a liquid medicament component to obtain a mixed medicament ready for delivery to the patient. A patient may be a human or animal, such as a cat, dog, horse, cow, sheep or pig.

The dry medicament component may for example be a lyophilized medicament component.

The reconstitution is normally performed just prior to the delivery of the medicament.

By medicament expelling process is meant the process of the moving the plunger rod from the retracted plunger rod position to the plunger rod extended position whereby medicament is expelled from the cartridge compartment through the cartridge outlet. The delivery of medicament to the patient may occur during this process.

The auto injector process may be a full injection cycle including at least a first plunger rod movement process, the medicament reconstitution process, the medicament expelling process, and a re-setting of the auto injector to an original position where the cartridge can be removed from the auto injector thereby allowing for insertion of a new cartridge in the auto injector.

In one or more examples, the predefined threshold value indicative of the electrical voltage needed for performing the auto injector process is set to a first fixed threshold value for temperatures above a predetermined threshold temperature, and to a second fixed threshold value for temperatures at or below the predetermined threshold temperature. In this manner, the auto injector makes a distinction between the battery voltage needed at high temperature and at low temperature. The first fixed threshold value may be indicative of the minimum electrical battery voltage level needed for performing an auto injector process at the predetermined threshold temperature. This ensures that if the auto injector is used at a temperature higher than the predetermined threshold temperature, there will always be sufficient battery voltage to perform the process, since the battery voltage needed to perform the auto injector process decreases with increasing temperature.

The second threshold value may be two times higher than the first threshold value. Thus, the second threshold value may be set to be equal to twice of the minimum electrical battery voltage level needed for performing an auto injector process at the predetermined threshold temperature. Thereby a higher requirement to the battery voltage level left in the battery is used when the temperature sensor measures low temperatures below the predetermined threshold temperature.

The second threshold value may be between 3000 MV and 4500 MV, such as between 3500 MV and 4000 MV, such as between 3800 MV and 3900 MV, such as 3850 MV.

The predetermined threshold temperature may be at or below 15 degrees Celsius, such as at or below 14 degrees Celsius, such as at or below 13 degrees Celsius, such as at or below 12 degrees Celsius. Depending on the viscosity of the medicament in the cartridge, adapted to be received in the auto injector, the predetermined threshold temperature may be higher than 15 degrees Celsius.

In one or more examples, the predefined threshold value increases when the measured temperature decreases. The predefined threshold value thereby take into account that the viscosity of the medicament increases with decreasing temperature, which is why a higher battery voltage level is needed to expel the medicament from the cartridge. Also, at lower temperatures, a higher battery voltage consumption will be present. These factors increase the battery voltage level needed to perform the auto injector process at lower temperatures.

In one or more examples, the processing unit is further configured to prevent initiating of the auto injector process if the measured temperature is at or below the predetermined threshold temperature.

In one or more examples, the initiation of the auto injector process only occurs if the calculated residual electrical battery voltage level is larger than the predetermined threshold value by at least a predetermined tolerance value. The tolerance value may also be seen as a safety margin to ensure that enough battery voltage is available should the temperature decrease further during the time, in which the auto injector perform the auto injector process.

The predetermined tolerance value may be at least 5%, such as at least 10%, such as at least 15%, such as at least 20%, such as at least 30%, such as at least 40%, such as at least 50%, such as at least 60%, such as at least 70%, such as at least 80%, such as at least 90%, such as at least 100% larger than the minimum electrical battery voltage level needed to perform the auto injector process at the measured temperature. Even higher tolerance values could also be imagined.

The predetermined tolerance value may increase with decreasing temperature. This accounts for situations where the battery voltage consumption is not increasing in a predictable manner as the temperature decreases.

In one or more examples, the cartridge further comprises a cartridge code feature, the cartridge code feature comprising information indicative of at least the medicament viscosity at at least one predefined temperature, and wherein the auto injector further comprises a cartridge code sensor coupled to the processing unit and configured to receive the information indicative of at least the medicament viscosity at at least one predefined temperature from the cartridge code feature when a cartridge is received in the auto injector. An examples of an information indicative of at least the medicament viscosity at at least one predefined temperature, is an information of the concentration of the medicament in the cartridge combined with an information of the type of medicament in the cartridge. When having information on both the medicament type and the concentration, the viscosity at at least one temperature, such as room temperature, may be known.

Different tolerance values may further be obtained based on the information of the medicament type in the cartridge. Thus, in situations where different medicaments are to be handled by the auto injector, the calculation of lowest acceptable battery voltage level may additionally include the knowledge about the medicament viscosity and/or the temperature dependent viscosity of the medicament. Possibly, this information about the possible medicaments to be delivered using the auto injector is provided in the cartridge code read by the auto injector upon loading the cartridge.

The code sensor may be configured to read a cartridge code feature, such as a cartridge code feature of the cartridge and/or attached to the cartridge. The code sensor may be configured to transmit a code signal indicative of the cartridge code feature. The code sensor may be configured to read the cartridge code feature in a plurality of positions. The cartridge code sensor may be movable. The cartridge code sensor may comprise a plurality of sensors, such as a plurality of transmitters and/or receivers.

The code sensor may comprise an optical sensor. The code sensor may comprise an optical sensor comprising a transmitter and a receiver, such as a light transmitter and a light receiver. The code sensor may be configured to read the cartridge code feature. The code sensor may be configured to read QR codes, bar codes, color codes, and/or any combination hereof. Accordingly, the cartridge code feature may be a QR code, bar code, color code and/or any combination hereof.

The processing unit may be coupled to the code sensor. The processing unit may be configured to receive from the code sensor a code signal indicative of the cartridge code feature.

In one or more examples, the predefined threshold value indicative of the minimum electrical battery voltage level needed for performing the auto injector process at the measured temperature is also depending on the medicament viscosity, and wherein the processing unit is further configured to:
receive information indicative of at least the medicament viscosity at at least one predefined temperature from the cartridge code feature;
obtain the predefined threshold value indicative of the minimum value of the electrical voltage needed for performing the auto injector process based both on the temperature as measured by the temperature sensor and on the information indicative of at least the medicament viscosity at at least one predefined temperature from obtained from the cartridge code feature.

The medicament viscosity is temperature dependent. The viscosity of medicament at any specific temperature may therefore be stored in the cartridge code feature as a curve.

In one or more examples, the auto injector further comprises a user interface coupled to the processing unit, wherein the processing unit is configured to instruct a user via the user interface to re-charge the battery if:
A) the difference between the calculated residual electrical battery voltage level and the predefined threshold value indicative of the minimum electrical battery voltage level needed for performing the auto injector process at the measured temperature is smaller than the predetermined tolerance value, or
B) the calculated residual electrical battery voltage level is smaller than the predefined threshold value at the measured temperature.

The user interface may comprise a plurality of LEDs including a first LED, wherein the user interface instructs the user to re-charge the battery by flashing the first LED. By flashing is meant that the first LED may continuously emit light having a first color. Alternative, the first LED may blink. The color of the light of the first LED may be red to indicate that the user need to pay attention to the battery level.

The first LED may flash until:
A) the difference between the calculated residual electrical battery voltage level and the predefined threshold value indicative of the minimum electrical battery voltage level needed for performing the auto injector process at the measured temperature is larger than the predetermined tolerance value, or
B) the calculated residual electrical battery voltage level is larger than the predefined threshold value at the measured temperature.

After the battery has been sufficiently re-charged, the first LED may stop flashing and a second LED may be turned on, e.g. emitting light having a different color than that of the first LED, e.g. a green color compared to a red color of the first LED. The flashing of the second color is meant to indicate to the user, that the battery has been sufficiently recharged for performing the auto injector process at the temperature measured by the temperature sensor.

In one or more examples, the battery calculation module is configured for calculating at least one of:
a time since the re-chargeable battery was last charged;
an age of the re-chargeable battery; and
an electrical voltage used since the re-chargeable battery was last charged; wherein the processing unit is configured to obtain an updated predefined threshold value indicative of the minimum electrical battery voltage level needed for performing the auto injector process at the measured temperature based on at least one of:
the time since the re-chargeable battery was last charged;
the age of the re-chargeable battery; and
the electrical voltage used since the re-chargeable battery was last charged.

When a battery ages, the performance often decreases. Taking the battery age into consideration may thus be relevant to ensure that the battery will provide enough voltage level to the drive module to allow the full auto injector process to be performed. Also, if it has been a long time since the battery was re-charged the last time, the voltage level available in the battery, may have decreased. Taking this into consideration together with a recording of previous voltage level consumptions, may also assist the battery calculation module in performing the most correct calculation of the remaining battery voltage level capacity in the battery.

A start-up method for determining whether battery voltage level is sufficiently high for the processing unit to allow the auto injector process to proceed may be run when the auto injector is turned on. The method comprises measuring the temperature of the auto injector, e.g. the ambient temperature, and/or the temperature close to the re-chargeable battery and/or the temperature indicative of the medicament temperature, by means of the temperature sensor. A measure of the battery voltage level is also measured in the same sequence by means of the battery calculation module.

The method further comprises determining if the temperature is above a predetermined threshold temperature, such as 15 degrees Celsius or such as 12 degrees Celsius. If the temperature is above the predetermined threshold temperature, the processing unit determines if there is sufficient electrical battery voltage level to perform an auto injector process. Such auto injector process may be at least one injection cycle. A tolerance value may possibly be added such that the sufficient electrical battery voltage level is set to include a threshold value. The determination is made by comparing the measured battery voltage level indicative of the residual battery voltage level, with the predefined threshold value needed to perform the auto injector process at the measured temperature.

If there is sufficient electrical battery voltage level to perform the auto injector process, the processing unit communicates to the user that the auto injector is ready for use, i.e. the user may perform the auto injector process.

If there is not sufficient electrical battery voltage level to perform the auto injector process, the processing unit communicates to the user that the battery needs to be recharged before the auto injector is ready for use, i.e. before the user may perform the auto injector process. Before the auto injector process can be initiated, the method is repeated.

If the temperature measured in the first step is determined to be below the predetermined threshold temperature, the processing unit determines if there is sufficient electrical battery voltage level to perform not one, but two auto injector processes, e.g. two full injection cycles. If there is sufficient electrical battery voltage level to perform two auto injector processes, the processing unit communicates to the user that the auto injector is ready for use, i.e. the user may perform the auto injector process.

If there is not sufficient electrical battery voltage level to perform two auto injector processes, the processing unit communicates to the user a need to re-charge the battery before the auto injector is ready for use, i.e. before the user may perform the auto injector process. Before the auto injector process can be initiated, the method is repeated. The temperature is normally not measured again till the devices makes a new self-test validation, i.e. runs the method again.

In addition or as an alternative, if the temperature is below the threshold temperature, e.g. below 10-15 degrees Celsius such as below 15 degrees Celsius, when the auto injector is turned on, the auto injector may be configured to prevent performing of any auto injector processes until the temperature is increased to a level threshold temperature, e.g. above the 15 degrees Celsius. The threshold temperature depends on the medicament which the auto injector is to deliver.

The reason for the auto injector to prevent an auto injector process to be performed at too low temperatures is that the medicament viscosity is too high and therefore the auto injector process requires more energy than the battery capacity calculation presumes. Further, the battery capacity decreases at lower temperatures.

In one or more examples, the auto injector further comprises a temperature control unit coupled to the processing unit, wherein the temperature control unit is configured to heat the auto injector, and wherein the processing unit is further configured to:
obtain the measured temperature measured by the temperature sensor;
initiate a heating of the auto injector if the measured temperature is below a predetermined heating-initiation temperature;
obtain a second measure of measured temperature measured by the temperature sensor;
stop the heating of the auto injector when the second temperature is above a predetermined heating-stop temperature and/or after the auto injector has been heated for a predetermined time.

By predetermined heating-stop temperature is meant a preset temperature, at which the temperature of the auto injector is considered acceptably high for the auto injector process to be performed. At the predetermined/preset heating-stop temperature, the viscosity of the medicament and/or the battery will normally be at temperature, where the amount of battery voltage required to perform the auto injector process is within what is considered normal/acceptable.

The heating of the auto injector may be a more local heating, such as a local heating of the cartridge/the medicament in the cartridge when a cartridge is inserted in the auto injector.

Thus, in one or more examples, the auto injector further comprises a temperature control unit coupled to the processing unit, wherein the temperature control unit is configured to heat the cartridge compartment containing the medicament when a cartridge is received in the auto injector, and wherein the processing unit is further configured to:
obtain a medicament temperature measured by the temperature sensor or a second temperature sensor;
initiate a heating of the cartridge compartment containing the medicament if the medicament temperature is below a predetermined heating-initiation temperature;
stop the heating of the cartridge compartment containing the medicament when the medicament temperature is above a predetermined heating-stop temperature and/or after the cartridge compartment containing the medicament has been heated for a predetermined time.

The cartridge may be made of glass, and/or polymer.

The cartridge may comprise a cartridge outlet, e.g. at a first cartridge end. The cartridge outlet may be configured for fluid communication with the compartment, e.g. at the first cartridge end. The cartridge may be configured to expel medicament through the cartridge outlet. The cartridge outlet may be configured to be coupled with a needle, such as a hypodermic needle, to provide the medicament to be expelled through the needle.

The cartridge may be a dual chamber cartridge. The cartridge compartment may have a first cartridge sub-compartment and a second cartridge sub-compartment. The first cartridge sub-compartment may be between the first stopper and the second stopper. The second cartridge sub-compartment may be between the second stopper and the cartridge outlet and/or the third stopper.

The first cartridge sub-compartment may contain a first medicament component of the medicament. The second cartridge sub-compartment may contain a second medicament component of the medicament. Each of the first medicament component and/or second medicament component may be a dry composition, a fluid, a liquid, a gel, a gas, and/or any combination thereof. The first medicament component and/or the second medicament component may be solute, such as a dry composition. The first medicament component and/or the second medicament component may be a solvent, such as a fluid composition, such as a liquid composition. The second medicament component may be a dry composition and the first medicament component may be a fluid composition, e.g. water or ethanol or saline solution or buffer solution or preservative solution. The second medicament component may be a solute. The first medicament component may be a solvent. It is envisaged that the medicament may be any medicament being injectable via a hypodermic needle, for example after reconstitution of the medicament.

The medicament may be a growth hormone. The medicament may be human growth hormone. Thus, the medicament may be made for the treatment of human growth hormone, hGH, however, this is only an exemplary use of the auto injector. The medicament may be a depot version, or hGH prodrug, such as a long-acting version, of human growth hormone. The medicament may be lonapegsomatropin. The second medicament component may be a dry composition of human growth hormone.

The cartridge may have a bypass section, e.g. for providing fluid communication between the first cartridge sub-compartment and the second cartridge sub-compartment, e.g. when the second stopper is positioned in the bypass section. The cartridge may have a plurality of bypass sections providing fluid communication between neighbouring cartridge sub-compartments, e.g. when a stopper separating the neighbouring cartridge sub-compartment is positioned in the respective bypass section.

The disclosed auto injector may be a reusable auto injector. A reusable auto injector may be especially useful when the cartridge comprises a plurality of sub-compartments. For example, an auto injector for a multi compartment or multi chamber cartridge may be more advanced, and therefore it may be beneficial to allow the auto injector to be used more than one time. For example, the auto injector may provide automated processes for mixing medicament components, such as for mixing medicament components initially provided in different sub-compartments of the cartridge.

The cartridge outlet may be an injection needle having an inner needle diameter. The predefined threshold value indicative of the electrical voltage needed for performing the auto injector process at the measured temperature may further depend on the inner needle diameter. The thinner the injection needle is, the higher is the force needed to eject the medicament from the cartridge compartment through the needle. Thinner injection needles are often desired since they introduce a lower amount of discomfort for the user. Also, there is normally a requirement for the auto injector to eject the medicament in a relative short amount of time, such as 15 seconds, 14 seconds, 13 seconds, 12 seconds, 11 seconds, 10 seconds or less. The combination of a thin injection needle and a short ejection time, adds requirements to the plunger rod force and thereby the battery voltage level needed for the drive module to drive the plunger rod. This information may therefore be included in the processing unit when it obtains the predefined threshold value.

In one or more examples, the inner needle diameter is between 145 μm and 160 μm, such as between 146 μm and 159 μm, such as between 147 μm and 158 μm, such as between 148 μm and 157 μm, such as between 149 μm and 156 μm, such as between 150 μm and 155 μm, such as between 151 μm and 154 μm, such as between 152 μm and 153 μm, such as e.g. 153 μm. The needle may for example be a 31 gauge needle.

The cartridge may comprise a first stopper movable inside the cartridge compartment, e.g. in a first stopper direction towards the first cartridge end. For example, the medicament may be expelled through the cartridge outlet upon movement of the first stopper, e.g. in the first stopper direction. Air may further be expelled from the cartridge, such as from the cartridge compartment, through the cartridge outlet.

The cartridge may comprise a cartridge back face, e.g. at a second cartridge end, such as opposite the cartridge outlet. The cartridge back face may comprise a cartridge back end opening. The cartridge back end opening may provide access for the plunger rod to the first stopper.

The cartridge receiver may be configured to receive the cartridge through a cartridge receiver opening. Thus, the cartridge may be inserted in the cartridge receiver through the cartridge receiver opening. The cartridge receiver may be configured to receive the cartridge through a cartridge receiver opening in a cartridge receiving direction. The cartridge receiving direction may be along the longitudinal axis.

In one or more examples, movement of the plunger rod from the retracted plunger rod position to a locking plunger rod position positioned between the retracted plunger rod position and the extended plunger rod position locks the cartridge inside the auto injector, wherein the processing unit is further configured for preventing movement of the plunger rod to the locking plunger rod position if:
the calculated residual electrical voltage of the re-chargeable battery is smaller than the value of the electrical voltage needed for performing the auto injector process at the measured temperature, or
the difference between the calculated residual electrical voltage of the re-chargeable battery and the value of the electrical voltage needed for performing the auto injector process at the measured temperature is smaller than the predetermined tolerance value.

In one or more examples, the auto injector further comprises:
a housing accommodating the cartridge receiver, the plunger rod, the drive module, the temperature sensor, the re-chargeable battery, the battery calculation module and the processing unit;
a connector opening in the housing allowing the auto injector to be connectable to an electrical voltage supply for re-charging the re-chargeable battery.

The electrical voltage supply may be a main power socket, a USB port, a laptop, and/or an external battery. The housing may accommodate a first electrical connector accessible via a connector opening in the housing. The first electrical connector may accept a second electrical connector of the electrical voltage supply.

The auto injector may further comprise:
an elongated ejector comprising an ejector member movable along a longitudinal axis between a first ejector position and a second ejector position and being configured to follow movement of the cartridge along the longitudinal axis when the cartridge is received in the cartridge receiver;
a blocking member coupled to the ejector member, the blocking member being configured to move between a blocking position wherein the connector opening is blocked and a non-blocking position wherein the connector opening is not blocked, wherein the blocking member is in the blocking position when the ejector member is in the second ejector position, and wherein the blocking member is in the non-blocking position when the ejector member is in the first ejector position.

This provides blocking of the connector opening, thereby preventing connection to an external electrical power supply, such as the main grid, when a cartridge is received in the auto injector. This further provides restriction of insertion of a cartridge if the auto injector is connected to an external electrical power supply, such as the main grid. The safety mechanism thereby provided in an auto injector reduces the risk of serious electric shock in users of the auto injector. This may prevent simultaneous presence of a needle and connection to an external electrical power supply.

Thus, a safety mechanism which, independently of the user chosen sequence, may thereby prevent simultaneous connection to an external electrical power supply, such as the main grid, e.g. via a charger, and usage of the auto injector for administering medicament.

Since the ejector member is configured to follow movement of the cartridge along the longitudinal axis, the insertion of the cartridge is determinant for whether or not the connector opening is blocked or not. Thereby a safety feature is provided, which is in particular advantageous for an auto injector for exchangeable cartridges, such as disposable cartridges and/or where a needle is attached to the cartridge prior to insertion of the cartridge into the auto injector.

The housing may have a connector opening. The connector opening may be a hole in the housing. The connector opening may be configured to allow passage of the second electrical connector, such as to allow access to the first electrical connector. The connector opening may be sized to the first and/or second electrical connector.

The battery may be configured to be charged by connection of the first electrical connector and the second electrical connector. The rechargeable battery of the auto injector may be a Li-ion battery or a NiCd battery or a NiMH battery.

The first electrical connector may accept the second electrical connector. The second electrical connector may electrically connect the first electrical connector to the electrical power supply. Connection of the first electrical connector and the second electrical connector may provide charging of the battery, such as by providing electrical power from the electrical power supply to the battery. The first electrical connector and/or the second electrical connector may be a USB compliant connector. The first electrical connector may be a female connector. The second electrical connector may be a male connector.

The auto injector may comprise an ejector comprising the ejector member. The ejector may be configured to eject the cartridge from the cartridge receiver.

The ejector member may have an ejector abutment face. The ejector abutment face may be configured to abut a face of the cartridge, such as the cartridge back face. The cartridge back face may abut the ejector abutment face upon insertion of the cartridge in the cartridge receiver. The ejector member may be moved towards the second ejector position, such as in the receiving direction, by insertion of the cartridge in the cartridge receiver, e.g. by movement of the cartridge back face in the receiving direction causing movement of the ejector abutment face in the receiving direction.

In one or more examples, in the first ejector position the cartridge is not in the auto injector. Thus, the ejector member may be in the first ejector position when the cartridge is not received in the cartridge receiver. The ejector member may be in the second ejector position when the cartridge is received in the cartridge receiver.

In one or more examples, the ejector member is spring-biased. Thus, the ejector member may be an ejector resilient member. When the cartridge is received within the auto injector, the ejector resilient member may be compressed and the blocking member is moved to the blocking position.

The auto injector and/or the ejector of the auto injector may comprise an ejector resilient member. The ejector resilient member may be configured to exert a force on the ejector member. The ejector resilient member may be configured to bias the ejector member towards the first ejector position, e.g. opposite the receiving direction.

The blocking member may be configured to close and/or block the connector opening. The blocking member is configured to move between a blocking position and a non-blocking position. In the blocking position the connector opening is blocked, e.g. access to the first electrical connector, such as for the second electrical connector, is prevented and/or restricted, and a non-blocking position wherein the connector opening is not blocked, e.g. access to the first electrical connector, such as for the second electrical connector, is allowed and/or not prevented and/or not restricted.

The blocking member may be movable by a translational movement between the blocking position and the non-blocking position. Alternatively or additionally, the blocking member may be movable by a rotational movement between the blocking position and the non-blocking position. The blocking member may be movable between the blocking position and the non-blocking position along the longitudinal axis. Alternatively, the blocking member may be movable between the blocking position and the non-blocking position perpendicular to the longitudinal axis. For example, the blocking member may be rotationally moved around the longitudinal axis between the blocking position and the non-blocking position.

The blocking member may be a door, such as a sliding door. The blocking member, e.g. in the blocking position, may completely block the connector opening. Alternatively, the blocking member, e.g. in the blocking position, may partially block the connector opening.

The blocking member may be configured to block the connector opening when a cartridge is received in the cartridge receiver. Alternatively or additionally, the blocking member may be configured to prevent insertion of a cartridge in the cartridge receiver when the first electrical connector and the second electrical connector are connected, such as when an electrical connector, such as the second electrical connector, is inserted through the connector opening. For example, the blocking member may be prevented to move to the blocking position if the first electrical connector is coupled to the second electrical connector. For example, the movement of the blocking member may be prevented by the first and/or second electrical connector, e.g. the first and/or second electrical connector may obstruct the path of movement of the blocking member towards the blocking position.

Insertion of the cartridge in the cartridge receiver may cause movement of the blocking member. For example, the blocking member may be coupled to the ejector member, such as to translate movement of the ejector member to the blocking member. Insertion of the cartridge in the cartridge receiver may move the ejector member, and movement of the ejector member may cause movement of the blocking member. Thus, insertion of the cartridge in the cartridge receiver may cause movement of the blocking member. Alternatively or additionally, the ejector member may be prevented to move to the second ejector position if the blocking member is prevented to move to the blocking position, e.g. if the first electrical connector is coupled to the second electrical connector. Thus, insertion of the cartridge in the cartridge receiver may be prevented if the first electrical connector is coupled to the second electrical connector.

The blocking member may comprise a first blocking coupling member. The ejector member may comprise a second blocking coupling member. The first blocking coupling member and the second blocking coupling member may be in engagement to translate movement of the ejector member to the blocking member. The first blocking coupling member may comprise a slot and/or a protrusion. The second blocking coupling member may comprise a protrusion and/or a slot. The second blocking coupling member and the first blocking coupling member may be movably connected. The second blocking coupling member and/or the first blocking coupling member may allow an amount of clearance, such that only part of the movement of the ejector is translated to movement of the blocking member.

Movement of the ejector member from a third ejector position to the second ejector position may move, and/or cause movement of, the blocking member from the non-blocking position to the blocking position. The third ejector position may be between the first ejector position and the second ejector position. For example, the ejector member may move from the first ejector position towards the second ejector position, such as upon insertion of a cartridge in the cartridge receiver, and from the third ejector position, located between the first ejector position and the second ejector position, the movement of the ejector member is transmitted to the blocking member, such that the blocking member moves towards the blocking position.

Alternatively or additionally, movement of the ejector member from a fourth ejector position to the first ejector position moves the blocking member from the blocking position to the non-blocking position. The fourth ejector position may be between the first ejector position and the second ejector position. The fourth ejector position may be the third ejector position. For example, the ejector member may move from the second ejector position towards the first ejector position, such as upon removal of the cartridge from the cartridge receiver, and from the fourth ejector position, located between the first ejector position and the second ejector position, the movement of the ejector member is transmitted to the blocking member, such that the blocking member moves towards the non-blocking position.

The second blocking coupling member comprising a slot and/or a protrusion and the first blocking coupling member comprising a protrusion and/or a slot may allow an amount of clearance and facilitate such exemplified transmission of movement.

The blocking member and/or the first blocking coupling member of the blocking member, may comprise a first blocking member stop and a second blocking member stop. For example, the first blocking coupling member may comprise a slot comprising the first blocking member stop and the second blocking member stop. The second blocking coupling member may comprise a protrusion arranged to catch the first blocking member stop by movement in one direction along the longitudinal axis, and arranged to catch the second blocking member stop by movement in another direction along the longitudinal axis. For example, the second blocking coupling member may catch the first blocking member stop upon movement of the ejector member towards the first ejector position, such as upon removal of the cartridge from the cartridge receiver. The second blocking coupling member may catch the second blocking member stop upon movement of the ejector member towards the second ejector position, such as upon insertion of the cartridge in the cartridge receiver.

Alternatively or additionally, the ejector member, and/or the second blocking coupling member of the ejector member, may comprise a first blocking member stop and a second blocking member stop. For example, the second blocking coupling member may comprise a slot comprising the first blocking member stop and the second blocking member stop. The first blocking coupling member may comprise a protrusion arranged to catch the first blocking member stop by movement in one direction along the longitudinal axis, and arranged to catch the second blocking member stop by movement in another direction along the longitudinal axis. For example, the first blocking coupling member may catch the first blocking member stop upon movement of the ejector member towards the first ejector position, such as upon removal of the cartridge from the cartridge receiver. The first blocking coupling member may catch the second blocking member stop upon movement of the ejector member towards the second ejector position, such as upon insertion of the cartridge in the cartridge receiver.

Providing such non-fixed coupling between the ejector member and the blocking member provides for a shorter device, as it converts a long sliding movement, e.g. of the ejector member, to a shorter one, e.g. of the blocking member.

Alternatively, the first blocking coupling member and the second blocking coupling member may be fixedly connected. For example, the ejector member and the blocking member are fixedly connected with respect to movement along the longitudinal axis.

Movement of the ejector member to the second ejector position may require movement of the blocking member to the blocking position. For example, if the blocking member is prevented from moving to the blocking position, e.g. if the second electrical connector is coupled to the first electrical connector, the movement of the ejector member to the second ejector position is restricted and/or impossible. Thereby it may be prevented that a cartridge is received by the cartridge receiver if the second electrical connector is connected, e.g. if a charger is connected to the auto injector to charge the battery.

In one or more examples, the auto injector further comprises an ejector lock configured for rotating at least a fraction of a revolution from an initial angular position to a first angular position when the plunger rod moves from the retracted plunger rod position towards the extended plunger rod position, wherein the rotation of the ejector lock retains the ejector member in a longitudinal and rotational position.

The ejector lock may be configured to restrict movement of the ejector member, such as along the longitudinal axis.

In one or more examples, the ejector member comprises an ejector support face that supports the cartridge and the cartridge holder if the cartridge is received in the cartridge receiver, wherein when the rotation of the ejector lock retains the ejector member in a longitudinal and/or rotational position, the cartridge and the cartridge holder are also retained in a longitudinal and/or rotational position.

In one or more examples, the blocking member remains in the blocking position during the auto injector process.

The drive module may be configured to receive electrical power from the battery. The drive module may be electrically connected to the battery for receiving electrical power. The drive module may be accommodated by the housing. The drive module may comprise a motor, such as an electro-mechanical motor, such as a DC motor, e.g. a DC motor with or without brushes. The drive module may comprise a solenoid motor. The drive module may comprise a shape memory metal engine. The drive module may comprise an arrangement of springs configured to actuate the plunger rod. The drive module may comprise a pressurized gas configured to actuate the plunger rod.

The cartridge receiver may comprise a cartridge receiver compartment configured to receive a cartridge assembly, with at least one cartridge retention member, when inserted through a cartridge receiver opening along a longitudinal axis in a receiving direction; wherein the cartridge receiver has a passage through which the at least one cartridge retention member travels at least in the receiving direction, and a member preventing movement beyond a retention position in a direction opposite of the receiving direction.

The elongated ejector may comprise a longitudinal ejector slot extending towards the ejector support face from an ejector rest portion. The elongated ejector may be suspended to move along the longitudinal direction and may be spring-loaded in the direction opposite of the receiving direction.

The ejector lock may be supported for turning at least a fraction of a revolution and maintained in a longitudinal position relative to the housing. The ejector lock may have an ejector lock support portion that is configured to align with and slide along the longitudinal ejector slot at a first angle and to be brought to align with the ejector rest portion at a second angle. Thereby the ejector rest portion and the ejector lock support member in combination may form a stop that is disengaged at the first angle and engaged at the second angle.

Thus, it is appreciated that, at the second angle, the ejector lock by its turning effectively introduces a stop, by its support member receiving the rest portion. The stop prevents further movement of the rest portion beyond the support member in the receiving direction, if the rest portion is not otherwise prevented from landing on the support member. Thus, the stop contributes to prevent the cartridge from movement beyond a stop position, relative to the housing, in the receiving direction.

At least when the needle of the cartridge assembly penetrates a patient's skin, a force is transferred from the needle to the cartridge and works to push the cartridge backwards in the receiving direction against the stop when it is engaged. The stop at least contributes to maintaining the position of the cartridge since otherwise precise dose administering may be obstructed.

When the ejector, by means of the stop, sits at the stop position, its ejector support face supports that an end portion of the cartridge or cartridge assembly may rest thereon to prevent unintended movement in the receiving direction.

The auto injector enables convenient front-loading of a cartridge accommodated in a cartridge assembly. Since the needle on the cartridge assembly may be protected by a needle cover reliably attached to the cartridge assembly, there is no increased risk of being injured by the needle while loading the auto injector with a cartridge accommodated in a cartridge assembly.

The auto injector enables convenient front-loading by: overcoming the spring-loaded bias when the cartridge meets the support face of the ejector, guiding the cartridge retention members from the passage to a position where the cartridge is prevented from moving out of the cartridge receiver, and turning the ejector lock to prevent movement beyond the stop in the receiving direction such that the cartridge maintains its position in the housing when a pressure on the needle in the receiving direction is at least partially transferred to the cartridge. It is important that the cartridge maintains its position since otherwise precise dose administering is obstructed.

It is appreciated that various distances should be dimensioned such that the support face of the ejector lock abuts the rest portion of the ejector when the cartridge sits in the retention position. Thereby the cartridge is locked or is in a locked position being prevented both from a forward movement and a backward movement, wherein the backward movement is the same as the receiving direction. The cartridge may be locked when the stop is engaged i.e. when the ejector lock is at the second angle, whereas the cartridge may be unlocked when the stop is disengaged i.e. when the ejector lock is at the first angle.

In one or more examples, the ejector lock support member extends axially from a wall of the ejector lock, e.g. in the form of a pin, to support the ejector at a transversely extending ejector rest portion. In one or more examples, the ejector lock support member extends transversely along a rim or edge of the ejector lock to support the ejector at transversely extending ejector rest portion or at an axially extending rest portion.

In one or more examples, one or more of the ejector lock support portion, the ejector rest portion and the ejector slot are recessed into the ejector lock or the ejector.

It is appreciated that the ejector lock is supported e.g. in a bearing that allows the lock to turn or be turned, at least a fraction of a revolution, while preventing a longitudinal movement.

In one or more examples, the ejector comprises an ejector rod with an ejector support face; wherein the ejector rod has an ejector rod bore to form a longitudinal passage, and wherein the ejector support face is arranged at one end of the ejector rod and has a disc shape or an annular shape. Thereby the disc shape or annular shape may form the support for the cartridge to rest thereon all around its end portion periphery. An aperture of the bore is located in a centre portion of the ejector abutment face. The bore gives room for a plunger that at least over some displacements thereof may move, independently of the ejector, to move a first stopper of the cartridge to expel at least a portion of the medicament from the cartridge.

In one or more examples, the ejector rod comprises an ejector collar arranged about the ejector support face. The ejector collar may have an inner diameter which is larger than an outer diameter of an end portion of the cartridge or cartridge assembly such that when the end portion of the cartridge or cartridge assembly abuts the ejector support face, the collar accommodates the end portion of the cartridge or cartridge assembly in a centralised position relative to the ejector rod. Thereby the cartridge or cartridge assembly can be guided to abut robustly on the ejector support face. In one or more examples, the ejector collar has an inner chamfer face which improves guiding of the cartridge or cartridge assembly towards the ejector support face.

In one or more examples, the ejector comprises an ejector rod configured with one or more ejector cut-outs to form one or more ejector cogs between the ejector cut-outs; and wherein the ejector lock is configured with one or more ejector lock cogs between one or more ejector lock cut-outs, respectively. Thereby the one or more ejector cogs may abut with the one or more ejector lock cogs to form the stop when engaged. The stop is engaged by aligning the cogs of the ejector and the ejector lock. Turning of the ejector lock at least a portion of a revolution about the longitudinal axis may disengage the stop whereby the ejector cogs can be accumulated in the ejector lock cut-outs. In this way the cogs and the cut-outs form complementary cogs and cut-outs.

The one or more cogs of the ejector rod and the one or more complementary cut-outs of the ejector lock are arranged at angular ranges about the longitudinal axis such that a cog can be accommodated in its entirety or partially by a complementary cut-out. A cog may extend over e.g. 45 degrees and a complementary cut-out may extend over 45 degrees plus an angular range to allow a clearance when the ejector rod and thus the cog moves in the longitudinal direction in or out of the cut-out of the ejector lock.

The angles at which cut-outs, cogs and complementary cogs and cut-outs are located implicitly define the first angular position where the ejector lock and ejector rod mutually are angularly positioned to allow movement of the cartridge assembly in the receiving direction, and the second angular position where the ejector lock and ejector rod mutually are angularly positioned to restrict movement of the cartridge assembly in the receiving direction at least restricted from moving beyond a predefined longitudinal position.

In the second angular position, at least one cog of the ejector rod abuts end-to-end at least one cog of the ejector lock; whereas in the first angular position, the at least one cog of the ejector rod is accommodated in a complementary cut-out.

The ejector cogs have end portions denoted an ejector rest portion and the ejector lock cogs have end portions denoted an ejector lock support portion. The ejector lock support portion supports the ejector rest portion when the stop is engaged.

Thus, the cogs have respective end portions which abut one another when the stop is engaged. Cut-outs of the ejector rod and cut-outs of the ejector lock have respective bottom portions. The bottom portions may extend between side portions separating cogs and cut-outs.

At a longitudinal position of the ejector rod where the cogs abut one another, the ejector defines by its length relative to the ejector lock at which position the cartridge or cartridge assembly is restricted from further movement by means of the stop in the receiving direction. The ejector may be suspended by a resilient member to move in the opposite direction of the receiving direction, in which case the cogs of the ejector rod travels away from the cogs of the ejector lock.

As mentioned above the cartridge or cartridge assembly may be supported at a disc shape or annular shape, which in one or more examples, is configured with a surrounding collar.

In one or more examples, the ejector rod has four cogs and four cut-outs and the ejector lock has four complementary cogs and four complementary cut-outs. This gives a good trade-off between the amount of rotation needed to turn the ejector rod and the ejector lock relative to each other from a securely locked position to an open position and mechanical robustness of the cogs.

In one or more examples, the cogs and cut-outs have an even angular size, e.g. 45 degrees or 60 degrees—in both cases minus an angular range to allow a clearance between a cog and a cut-out.

In one or more examples, one or more of the cut-outs and the cogs have a substantially rectangular shape.

Thus, the one or more cut-outs, such as ejector cut-outs and/or ejector lock cut-outs have edges orthogonal to the longitudinal axis and edges along the longitudinal axis. Due to the edges along the longitudinal axis a good engagement for retaining a relative angular position between the ejector rod and the lock is obtainable at least when the stop is engaged.

In one or more examples, the ejector cut-outs accommodate ejector lock cogs and ejector lock cut-outs accommodate ejector cogs in a complementary manner such that spaces are substantially filled out between cogs to resemble a cylindrical object. However, an angular clearance is typically needed between cogs to allow sufficiently low friction of a longitudinal movement and to allow for variations occurring during manufacture of the ejector and ejector lock.

In one or more examples, the cut-outs and the cogs comprise a triangular portion, an arc of a circle or another polygon or curve.

In one or more examples, the one or more cut-outs and the one or more cogs comprise a portion that is inclined relative to the longitudinal axis and relative to an axis orthogonal to the longitudinal axis.

The portion that is inclined relative to the longitudinal axis may be one or more of an end portion of one or more cogs, a bottom portion of one or more cut-outs, and a side portion of one or more cogs or cut-outs.

As an aspect of securing smooth longitudinal movement any angle between a side portion and a bottom portion should be 90 degrees or greater and any angle between a side portion and an end portion should be 90 degrees or greater. Thereby it should be prevented that a nose or overhang is formed, behind which cogs could be stuck in unfortunate positons.

In some of the aspects, wherein a portion is inclined, the ejector cut-outs may accommodate ejector lock cogs and ejector lock cut-outs may accommodate ejector cogs in a complementary manner such that spaces are substantially filled out between cogs to resemble a cylindrical object. However, an angular clearance is typically needed between cogs to allow sufficient low friction of a longitudinal movement.

In one or more examples, ejector cogs and lock cogs comprise an end portion that is inclined relative to the longitudinal axis at an angle of less than 40 degrees or less than 30 degrees or less than 20 degrees relative to the orthogonal of the longitudinal axis.

The inclined end portions may contribute such that when the stop is engaged by turning of the lock, further turning of the lock causes a longitudinally tightening force acting on the ejector and transmitted though the ejector to the cartridge and/or cartridge assembly. In this way it may be possible to at least substantially remove clearances that occur due to manufacture variations.

The end portions of lock cogs and end portions of ejector cogs are inclined substantially at the same angle such that the end portions are mutually parallel. In one or more examples, the bottom portions of the cut-outs may be inclined by substantially the same angle. Thereby the cogs fit into the cut-outs.

Thus the cogs have respective end portions which abut one another when the stop is engaged. Due to the inclined end portions and possibly manufacture variations, a desired tightening force or clearance reduction may occur at an angular position of the lock where the ejector cogs and the lock cogs do not align centre-to-centre, but somewhat offset therefrom.

Thereby it may be possible to tighten up the cartridge in a more precise longitudinal position as well as ensuring that cartridge length dimension tolerances do not result in or at least risk expelling fluid from the cartridge when pressing the needle towards the skin. Thus the risk of unintended rearward travel of the cartridge due to short cartridge length in combination with a too retracted lock position is reduced. Had the cartridge not been prevented from such unintended rearward travel, a plunger rod in contact with the stopper could possibly press out medicament too soon before the needle is properly inserted in the skin of the patient and thus the full dose would not be administered to the patient.

In one or more examples, the ejector comprises an ejector rod configured with a substantially cylindrical portion with one or more chamfers about the longitudinal axis to form a rotational asymmetric end portion; and wherein the ejector lock is configured with one or more complementary chamfer cuts to form a complementary rotational asymmetric end portion.

In one or more examples, the chamfer is positioned such that the end portion extends over a portion that is substantially orthogonal to the longitudinal axis. The end portion that extends over a portion that is substantially orthogonal to the longitudinal axis may extend over less than 180 degrees e.g. over less than 120 degrees or less than 90 degrees.

In one or more examples, the auto injector comprises a plunger rod; wherein the ejector comprises an ejector rod which is spring-loaded by a spring ejector member; wherein the plunger rod and the bore are configured for longitudinal relative movement. Thereby the ejector and the plunger rod can be closely integrated. Also, the plunger rod may be moved along the longitudinal axis, at least over some distance, without bringing the ejector rod along and vice versa.

The ejector rod may comprise a cylindrical object through which the bore extends; wherein the above-mentioned cut-outs and cogs are located at one end of the cylindrical object and wherein the above-mentioned disc shape or annular shape is arranged at the other end.

In one or more examples, the bore through the ejector rod and an exterior face of the plunger rod are configured with coupling means and complementary coupling means, respectively, that retains a relative angular position and allows a relative longitudinal movement. There may be formed a track extending along a straight line along the longitudinal axis on the wall of the bore that engages with a groove in the plunger rod, and/or vice versa. Thereby longitudinal movement is allowed while angular retention is provided.

In one or more examples, the plunger rod comprises an inner plunger rod part and an outer plunger rod part; wherein the inner plunger rod part and the outer plunger rod part are coupled by a thread; wherein the inner plunger part is retained in a bearing allowing rotation of the inner plunger part while preventing a longitudinal movement; and wherein the outer plunger rod part is retained in an angular position relative to the housing.

Thereby the outer plunger part may be actuated to move in the longitudinal direction by rotation of the inner plunger rod part. The outer plunger part may be configured to move a first stopper of the cartridge to expel at least a portion of the medicament from the cartridge.

In one or more examples, the inner plunger rod part is rotational driven by a drive module, which may comprise a motor and one or more of transmission and gearing to couple the motor to the inner plunger rod. The inner plunger rod part may comprise a spindle portion that engages with an inner thread in the outer plunger rod part.

In one or more examples, the outer plunger rod part is retained in the angular position relative to the housing in that there is formed a longitudinally extending plunger rod groove in the wall of the outer plunger rod; wherein the plunger rod groove engages with a longitudinally extending bead or track on the inner wall of the ejector rod bore. This configuration allows displacement, at least over some distances, of the outer plunger rod relative to the ejector rod and vice versa while retaining a relative angular position between them.

In one or more examples, the auto injector comprises an angle retaining slot and an angle retaining guide configured to engage with each other and arranged on or in the cartridge receiver or a member rigidly coupled to the cartridge receiver and at the ejector rod.

Thereby the ejector rod is suspended for angular retention with the cartridge receiver and for longitudinal displacement at least over some distances.

In one or more examples, the angle retaining slot is configured in a member that accommodates the ejector rod and plunger rod when in a retracted position; the angle retaining slot may sit next to the ejector rod when it is retracted. The member, which may accommodate a motor coupled to drive the inner plunger rod, may comprise a collar providing a curb or seat for a spring inflicting the spring-load on the ejector. The angle retaining guide is then arranged on the ejector rod.

In one or more examples, the ejector lock comprises an ejector lock guide pin configured to engage with a plunger rod track provided in the plunger rod, such that longitudinal movement of the plunger rod, at least over a predefined range, inflicts a turning of the ejector lock about the longitudinal axis.

Thereby it is possible to drive the auto injector with a single motor which at some displacements of the plunger rod inflicts turning of the lock and at other displacements of the plunger rod inflicts expel of a dose of medicament from the cartridge. At least the plunger rod track may be configured such that the plunger rod turns the ejector lock and disengages the stop at retracted positions where the plunger rod is at a position at a distance from the cartridge. The plunger rod track may be configured such that stop is engaged at advanced positions of the plunger rod, when at least it abuts or presses on a stopper of the cartridge.

In one or more examples, movement of the plunger rod in the receiving direction, at least over a range of longitudinal positions, inflicts a turning of the ejector lock to the first angular position. Thereby the stop is disengaged at retracted positions of the plunger rod.

In one or more examples, the ejector lock is configured with an ejector lock bore to accept at least an end portion of the outer plunger rod and an ejector lock guide pin that extends inwardly from a wall of the ejector lock bore; wherein the outer plunger rod is configured with a plunger rod track that engages with the ejector lock guide pin and extends from a plunger rod distal rim towards the cartridge assembly opening; wherein the plunger rod track has at least one track portion that leads the ejector lock guide pin from a first angle to a second angle that are angularly spaced apart to turn the ejector lock from the first angular position to the second angular position.

Thereby rotation of the inner plunger rod part may bring about a longitudinal movement of the outer plunger rod part to at least engage with the cartridge at least at some longitudinal positions of the outer plunger rod part and to inflict a rotation of the ejector lock at other longitudinal positions of the outer plunger rod part to either lock or unlock the ejector member rod.

In one or more examples, the track is configured such that it rotates the ejector lock via the guide pin to a position that unlocks the ejector rod when the outer plunger rod is at an extreme longitudinal position away from the cartridge receiver opening. The track may rotate the ejector lock via the guide pin to another position that locks the ejector rod when the outer plunger rod is at a less extreme longitudinal position away from the cartridge receiver opening. Thus, as the outer plunger rod member is moved from an extreme position in the direction opposite of insertion, the ejector lock moves from a position where the ejector is unlocked to a position where the ejector is locked.

It is appreciated that the angular position of the guide pin relative to the cogs and cut-outs and the angular position of the outer plunger rod relative to the ejector rod are synchronized such that the rotation inflicted to the ejector lock by the track via the guide pin is angularly positioned to allow the cogs to abut end-to-end in second angular position, and the cogs to be accommodated in the cut-outs in the first angular position. It is appreciated that in the first angular position, the ejector is unlocked by the ejector lock and in the second angular position the ejector is locked by the ejector lock.

In one or more examples, the length of the ejector member and ejector lock when adjoining each other such that the cogs are accommodated by the complementary cut-outs.

The ejector lock is arranged e.g. in a bearing that allows the lock to turn or be turned, at least a fraction of a revolution, while preventing a longitudinal movement.

In one or more examples, the at least one track portion that leads the guide pin from the first angle to the second angle is inclined about 40-50 degrees relative to the longitudinal axis.

In one or more examples, the at least one track portion that leads the guide pin from the first angle to the second angle is an intermediate portion that continues from a first longitudinally extending track portion and continues to second longitudinally extending track portion. In one or more examples, thereof the first track portion is wider than the second track portion. In one or more examples, wherein the first track portion is wider than the second track portion, the first track portion may comprise an inclined guide face or chicane that guides the guide pin into the intermediate track portion. The first track portion may extend from the first angle to the second angle. The inclined guide face may be inclined about 40-50 degrees relative to the longitudinal axis. The track is generally configured with track portions that extends longitudinally or at steep inclination angles; such that in the latter case, a turn is smoothly inflicted the guide pin typically by inclination angles not closer to the orthogonal of the longitudinal direction than about 30 degrees. Thereby, at least for that reason, the guide pin will not be stuck in the track.

In one or more examples, the track has a width at least at a portion of the track which is equal to a dimension of the guide pin plus a clearance, wherein the dimension of the guide pin may be its diameter or diameter or a circumferential circle or a width of the pin. The clearance may be smaller than 50% or smaller than 20% or smaller than 10% the dimension of the guide pin.

The first, entry, portion of the plunger rod track may be wider than the intermediate and second portion of the plunger rod track. Thereby the ejector lock guide pin may be received and guided into the track at wider angles. This may prevent the ejector lock guide pin from unintentionally obstructing movement of the plunger rod.

In one or more examples, the plunger rod track is configured as a recess that extends from the plunger rod distal rim of the outer plunger rod. The depth of the recess matches a length of the guide pin such that they engage sufficiently for turning the ejector lock.

In one or more examples, the ejector lock is coupled to a resilient member that biases the ejector lock towards the second angular position. Thereby more retracted positions of the plunger rod are required to disengage the ejector lock. This is particularly useful when an entry portion of the plunger rod track accepts the ejector lock guide pin at wider angles.

In some examples, the auto injector comprises a drive module with a motor and one or more of transmission and gearing to couple the motor to the inner plunger rod. The drive module may be power supplied from one or more of a battery and a power supply. The drive module may be controlled via a microprocessor programmed to control the plunger rod via the drive module in response to user activated controls such as push-buttons.

The auto injector may further comprise a resistance sensor. The resistance sensor may be configured to provide a resistance signal indicative of resistance against movement of the plunger rod. The processing unit may be coupled to the resistance sensor.

The processing unit may further be configured to:
control the drive module to move, such as advance, the plunger rod towards the extended plunger rod position with a plunger rod speed;
determine plunger rod position;
receive the resistance signal; and control the drive module to adjust movement of the plunger rod if the resistance signal is indicative of resistance against movement of the plunger rod above a high resistance threshold.

The high resistance threshold may be based on the plunger rod position.

The housing may accommodate the resistance sensor.

Also disclosed herein is a method for controlling an auto injector. The method comprises:
receiving a cartridge comprising a first stopper;
moving a plunger rod towards an extended plunger rod position with a plunger rod speed;
determining plunger rod position;
receiving a resistance signal indicative of resistance against movement of the plunger rod; and
adjusting movement of the plunger rod if the resistance signal is indicative of resistance against movement of the plunger rod above a high resistance threshold, wherein the high resistance threshold is based on the plunger rod position.

In this way is obtained an optimizing dosing accuracy through more fully emptying a drug cartridge during injection by applying more force to the stopper(s)—and maintain such elevated force over a period of time—thereby forcing deformation/compression of the stopper to better contact with (fill out) the interior cartridge shoulder area and thereby press out residual drug sitting here. Additionally is also provided an improved medicament utilization as less medicament may be wasted from each cartridge.

Plunger rod speed may further be optimized, e.g. leading to an optimization of the time of the injection procedure, e.g. time needed to inject the medicament and/or in preparing for injection. Further, patient safety is increased, e.g. by decreasing the risk of incorrect dosage of medicament.

An improved precision of medicament usage is additionally obtained, which allows for reducing the amount of medicament not being used. Thus, the cost of not used medicament may be reduced.

The high resistance threshold may be based on the plunger rod position. The high resistance threshold may be a first high resistance threshold and/or a second high resistance threshold and/or third high resistance threshold.

The processing unit may be configured to determine the high resistance threshold, e.g. based on the plunger rod position. The high resistance threshold may be a first high resistance threshold when the plunger rod position is between the retracted plunger rod position and a first plunger rod position. Alternatively or additionally, the high resistance threshold may be a second high resistance threshold when the plunger rod position is between a second plunger rod position and the extended plunger rod position.

The second high resistance threshold may be higher than the first high resistance threshold. When the second high resistance threshold corresponds to an extended plunger rod position at the end of the injection of the medicament, the high resistance threshold may be higher in order to ensure effective emptying of the cartridge without the risk of leakage at the stoppers or the septum at the end of injection because of the lower needle flow resistance contribution to the pressure in the cartridge.

The first high resistance threshold may be between 50-80 N, such as 50 N, 55 N, 60 N, 65 N, 70 N, 75 N, or 80 N. In an example, the first high resistance threshold is 55 N.

The second high resistance threshold may be between 70-100 N, such as between 75-85 N, or such as between 80-90 N, or such as 70 N, 75 N, 80 N, 85 N, or 90 N. In an example, the second high resistance threshold is 80 N.

The high resistance threshold may be a third high resistance threshold when the plunger rod position is between the first plunger rod position and the second plunger rod position. The high resistance threshold may be the third high resistance threshold when the plunger rod position is at a third plunger rod position. The third plunger rod position may be between the first plunger rod position and the second plunger rod position.

The third high resistance threshold may be higher than the first high resistance threshold. The third high resistance threshold may be lower than the second high resistance threshold. The third high resistance threshold may be between the first high resistance threshold and the second high resistance threshold.

The high resistance threshold, e.g. the third high resistance threshold, may be increasing as the plunger rod position is moved from the first plunger rod position to the second plunger rod position.

The distance between the extended plunger rod position and the first plunger rod position may be between 1-3 mm, such as 2 mm.

The distance between the retracted plunger rod position and the first plunger rod position may be between 0-60 mm.

The distance between the retracted plunger rod position and the first plunger rod position may be between 50-60 mm, such as 55 mm, 56 mm, or 57 mm.

The resistance sensor may be configured to measure pressure and/or force applied to a plunger rod front end of the plunger rod. The plunger rod front end may be configured to engage with the first stopper of the cartridge. The resistance sensor may be configured to measure pressure and/or force between the plunger rod and the stopper. For example, the resistance sensor may comprise a pressure transducer and/or a force transducer on the plunger rod front end. The plunger rod may comprise the resistance sensor.

Alternatively or additionally, the resistance sensor may be configured to determine electrical current through the drive module, and/or configured to determine electrical power consumed by the drive module. For example, the resistance sensor may be configured to measure electrical resistance, electrical current, and/or electrical voltage of the drive module. The resistance sensor may comprise an electrical resistance sensor, an electrical current sensor, and/or an electrical voltage sensor. The resistance signal may be based on electrical power consumed by the drive module, such as on the determined electrical power consumed by the drive module. The resistance signal may be based on electrical current through the drive module, such as on the measured electrical current through the drive module. The drive module may comprise the resistance sensor.

Instead of applying a dedicated force sensor, e.g. due to cost and architectural complexity of applying such a force sensor between a plunger and a cartridge stopper, a practical way to monitor equivalent plunger force and/or resistance may be through monitoring the current through the drive module, such as through the motor of the drive module. For electromechanical systems, this will correlate well to output force. The force acting upon an inductor inside a magnetic field can be expressed as F=B*I*l, where B is the magnetic field strength, I is the inductor current and l is the length of the inductor in the magnetic field.

The plunger rod position, such as a present plunger rod position, such as the plunger rod position at a specific moment in time, may be determined, e.g. by the processing unit. The plunger rod position may be determined based on detection from a sensor, such as a plunger rod position sensor.

The auto injector may comprise the plunger rod position sensor. The plunger rod position sensor may be configured to detect the position of the plunger rod and/or the position of the first stopper. The drive module may comprise the plunger rod position sensor.

The auto injector may comprise a tachometer. The plunger rod position sensor may comprise the tachometer. The plunger rod position sensor may be a tachometer. The tachometer may be configured to count the revolutions of the drive module, such as a motor of the drive module, such as the revolutions of the drive module from a set point, such as a point wherein the position of the plunger rod is known, such as the retracted plunger rod position, such as a fully retracted position of the plunger rod. The count of revolutions of the drive module may be used to determine the plunger rod position, i.e. the position of the plunger rod at a specific moment in time.

The tachometer may be configured to provide a tachometer signal indicative of a count of revolutions of the drive module. The processing unit may be coupled to the tachometer. The processing unit may be configured to receive the tachometer signal. The processing unit may be configured to determine the present plunger rod position based on the tachometer signal.

The processing unit may be coupled to the plunger rod position sensor. The processing unit may receive from the plunger rod position sensor a first plunger rod position sensor signal, such as the tachometer signal, indicative of the count of revolutions of the drive module. The processing unit may determine the position of the plunger rod based on the first plunger rod position sensor signal, e.g. the tachometer signal. The processing unit may receive a second plunger rod position sensor signal, e.g. from the plunger rod position sensor, indicative of the plunger rod being in a known position, such as in the retracted plunger rod position, such as a fully retracted position. The processing unit may be configured to determine the position of the plunger rod based on the first plunger rod position sensor signal, e.g. the tachometer signal, and the second plunger rod position sensor signal. The processing unit may be configured to determine the plunger rod position based on the tachometer signal and the retracted plunger rod position. For example, the processing unit may be configured to determine the plunger rod position based on the number of revolutions of the drive module since the plunger rod was in the retracted plunger rod position.

Adjusting the movement of the plunger rod may comprise decreasing the plunger rod speed.

Adjusting the movement of the plunger rod may comprise stopping the movement of the plunger rod.

Adjusting the movement of the plunger rod may comprise preventing movement of the plunger rod towards the retracted plunger rod position for a dwell time. Alternatively or additionally, adjusting the movement of the plunger rod may comprise maintaining the position of the plunger rod for a dwell time. Preventing retraction or movement towards the retracted plunger rod position may prevent back flow of medicament due to lowering of the pressure inside the cartridge.

Adjusting the movement of the plunger rod may comprise moving the plunger rod to the retracted plunger rod position. For example, the plunger rod may be moved to the retracted plunger rod position after the dwell time.

Adjusting the movement of the plunger rod may comprise gradually decreasing the plunger rod speed, stopping the plunger rod speed, preventing movement of the plunger rod towards the retracted plunger rod position, and moving the plunger rod to the retracted plunger rod position after the dwell time.

The movement of the plunger rod may be readjusted after adjusting the movement of the plunger rod. The processing unit may be configured to control the drive module to readjust the movement of the plunger rod after adjusting the movement of the plunger rod. For example, the movement of the plunger rod may be readjusted after adjusting the movement of the plunger rod if the resistance against movement of the plunger rod is below the high resistance threshold. The processing unit may be configured to control the drive module to readjust the movement of the plunger rod after adjusting the movement of the plunger rod, if the resistance signal is indicative of resistance against movement of the plunger rod below the high resistance threshold. Readjusting the movement of the plunger rod may comprise increasing the plunger rod speed.

The plunger rod speed may be varied. For example, the plunger rod speed may be based on the plunger rod position. The plunger rod speed may be a first plunger rod speed when the plunger rod position is between the retracted plunger rod position and a fourth plunger rod position. The plunger rod speed may be a second plunger rod speed when the plunger rod position is between a fifth plunger rod position and the extended plunger rod position. The second plunger rod speed may be lower than the first plunger rod speed. Alternatively, the second plunger rod speed may be higher than the first plunger rod speed. The processing unit may be configured to determine the plunger rod speed, e.g. based on the plunger rod position.

The fourth plunger rod position may be the first plunger rod position. The fifth plunger rod position may be the second plunger rod position. The first plunger rod position and the second plunger rod position may be the same plunger rod position. The fourth plunger rod position and the fifth plunger rod position may be the same plunger rod position.

The cartridge, such as the cartridge configured to be received by the auto injector, such as by the cartridge receiver of the auto injector, may have a cartridge outlet at a first cartridge end. The cartridge may comprise a cartridge back face, e.g. at the second cartridge end, such as opposite the cartridge outlet. The cartridge back face may comprise a cartridge back end opening. The cartridge back end opening may provide access for a plunger rod, such as the plunger rod of the auto injector, to the first stopper.

The cartridge compartment may contain a medicament. The cartridge outlet may be configured for fluid communication with the cartridge compartment, e.g. at the first cartridge end. The cartridge may be configured to expel medicament through the cartridge outlet. The cartridge outlet may be configured to be coupled with a needle, such as a hypodermic needle, to provide the medicament to be expelled through the needle.

The first stopper of the cartridge may be movable inside the cartridge compartment. The cartridge may comprise a second stopper movable inside the cartridge compartment. The second stopper may be between the first stopper and the cartridge outlet. The cartridge may comprise a third stopper movable inside the cartridge compartment. The third stopper may be between the second stopper and the cartridge outlet. The first stopper, the second stopper, and/or the third stopper may be movable inside the cartridge compartment towards the cartridge outlet, e.g. in a first stopper direction, such as towards a first cartridge end. For example, the medicament may be expelled through the cartridge outlet upon movement of the first stopper, the second stopper, and/or the third stopper, e.g. in the first stopper direction and/or towards the cartridge outlet.

It is envisaged that any embodiments or elements as described in connection with any one aspect may be used with any other aspects or embodiments, mutatis mutandis.

### BRIEF DESCRIPTION OF THE FIGURES

The above and other features and advantages of the present invention will become readily apparent to those skilled in the art by the following detailed description of exemplary embodiments thereof with reference to the attached drawings, in which:

FIG. 1 shows an exemplary auto injector;

FIGS. 2-3 show an exemplary auto injector with an exemplary cartridge as seen from two different directions;

FIG. 4 shows an exemplary auto injector with an electrical connector;

FIGS. 5A-B schematically illustrate parts of an exemplary auto injector;

FIGS. 6A-D schematically illustrate insertion and removal of an exemplary cartridge in an exemplary auto injector;

FIGS. 7A-F schematically illustrate an exemplary coupling between a blocking member and an ejector member;

FIGS. 8A-B schematically illustrate an exemplary blocking member;

FIG. 9 schematically illustrates an exemplary drive module and plunger rod; and

FIG. 10 schematically illustrates exemplary components of an exemplary auto injector.

FIG. 11 schematically illustrates an exemplary cartridge;

FIG. 12 shows an exemplary cartridge holder with a cartridge;

FIG. 13 shows a cross section of an exemplary cartridge assembly with a needle assembly;

FIG. 14 shows an exemplary cartridge receiver;

FIG. 15 shows an exemplary cartridge receiver with an ejector;

FIG. 16A shows a detailed view of the first section and second section of an exemplary cartridge receiver compartment; and FIG. 16B shows an inbound journey and an outbound journey of an exemplary cartridge retention member;

FIGS. 17A-17B are cross-sectional views of a first section and second section of an exemplary cartridge receiver compartment;

FIG. 18 shows a detailed view of alternative first section and second section of an exemplary cartridge receiver compartment;

FIG. 19 shows an exemplary outer plunger rod;

FIG. 20 shows an exemplary ejector;

FIG. 21 shows an exemplary ejector lock;

FIGS. 22A-22D show various positions of an exemplary ejector relative to an exemplary ejector lock;

FIG. 23 shows a cross section of an exemplary system comprising an auto injector and a cartridge assembly;

FIGS. 24A-D show cross sections of a portion of an exemplary system comprising an auto injector and a cartridge assembly; and

FIG. 25A-B show shows various positions of an exemplary ejector relative to the ejector lock in an embodiment where the cogs have inclined faces.

FIG. 26 shows a block diagram of an exemplary auto injector;

FIG. 27 schematically illustrates an exemplary auto injector;

FIGS. 28A-F show an exemplary graph of resistance threshold vs. plunger position;

FIG. 29 shows an exemplary graph of resistance vs plunger position;

FIGS. 30A-E show an exemplary graph of plunger speed vs. plunger position;

FIG. 31 shows a flow chart of an exemplary method;

FIG. 32 shows a flow chart of an exemplary method;

FIG. 33 shows a flow chart of an exemplary method;

FIG. 34 shows a flow chart of an exemplary method;

FIG. 35 shows a flow chart of an exemplary method;

FIG. 36 shows a block diagram of an exemplary auto injector;

FIG. 37 shows a flow chart of an exemplary method;

FIG. 38 shows a flow chart of an exemplary method;

FIG. 39 shows the temperature dependence of the viscosity of lonapegsomatropin in a medicament solution concentration of 22.0 mg/mL hGH; and

FIG. 40A and FIG. 40B show an exemplary measure of the injection force 1250 needed as a function of the plunger rod position when the cartridge contains, respectively, a 13.3 mg hGH/mL and a 5.2 mg hGH/mL lonapegsomatropin medicament solution.

### DETAILED DESCRIPTION

Various embodiments are described hereinafter with reference to the figures. Like reference numerals refer to like elements throughout. Like elements will, thus, not be described in detail with respect to the description of each figure. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

The term ‘user’ refers to a human being using the auto injector for self-administering a medicament. In this respect the user may also be designated a ‘patient’. Thus, one use case of the auto injector is self-administration of a medicament. The auto injector is described with this use case in mind. However, in another use case an assistant, e.g. a nurse or home carer, may operate the auto injector to administer the medicament into the patient. The latter use case is also enabled by the present disclosure of the auto injector. The user may use the auto injector in connection with his or her daily activities.

Throughout, the same reference numerals are used for identical or corresponding parts.

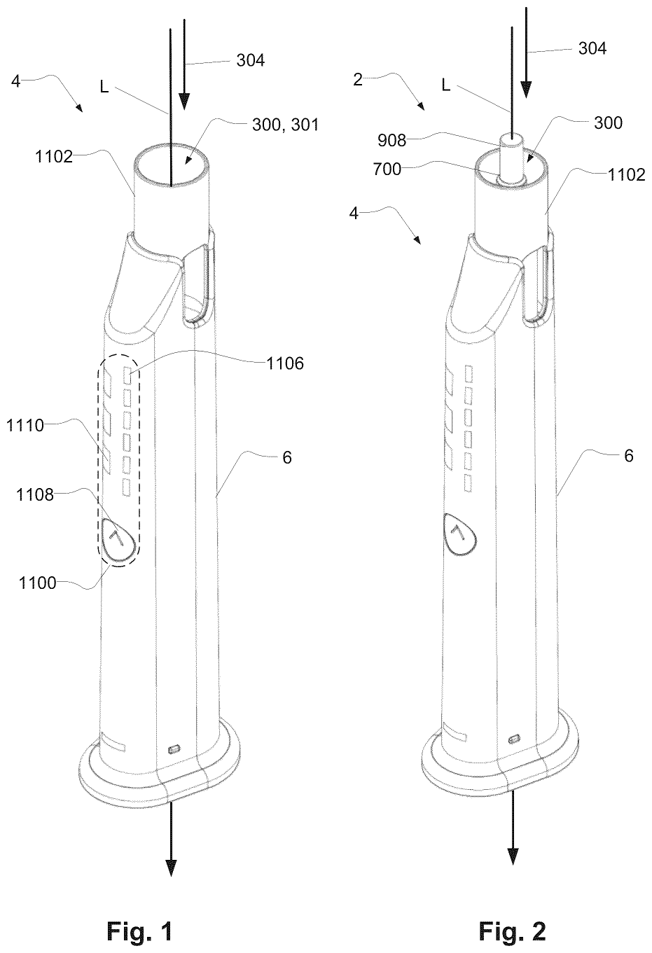

FIG. 1 shows an exemplary auto injector. The auto injector 4 may be configured for administering a medicament. The auto injector 4 may be an electronic auto injector, e.g. the auto injector 4 may be connectable to an electrical power supply (not shown), such as an external battery or a power plug.

The auto injector 4 comprises a housing 6. The auto injector 4 comprises a cartridge receiver 300. The cartridge receiver is configured to receive a cartridge and/or a cartridge assembly comprising a cartridge. The cartridge may contain the medicament.

The cartridge receiver 300 has a cartridge receiver opening 301. The cartridge receiver 300 is configured to receive the cartridge and/or the cartridge assembly through the cartridge receiver opening 301 in a cartridge receiving direction 304 along a longitudinal axis, L.

The auto injector 4 may comprise a user interface 1100, as illustrated. The auto injector 4 comprises a trigger member, such as the contact member 1102. The contact member 1102 may be configured to be pressed against an injection site on a patient's skin. The contact member 1102 may be movable in the cartridge receiving direction 304, relative to the housing, if pressed against the injection site. The contact member 1102 may be part of the user interface 1100.

The user interface 1100 comprises a first input member 1108, e.g. a button. The first input member 1108 may provide for a user input from a user. For example, the first input member 1108 may be used for receiving a push from a user to proceed to a next step.

The user interface 1100 comprises a first output member 1110 as illustrated, e.g. a plurality of LEDs. The first output member 1110 may provide for a user output to a user. The user interface 1100 may comprise a second output member (not shown), e.g. a speaker. The second output member may be configured to provide audible output to the user. For example, the first output member 1110 and/or the second output member may be used to indicate a step in the procedure to the user and/or to indicate an error message.

The auto injector 4 may comprise a cover (not shown) to protect the auto injector from dirt and filth when not in use.

The user interface 1100 may comprise a first LED 1106, which flashes when the battery needs recharging. By flashing is meant that the first LED 1106 may continuously emit light at a specific color. Alternative, the first LED 1106 may blink. The color of the light from the first LED 1106 may be red to indicate that the user need to pay attention to the battery level.

The first LED 1106 may flash until:
A) the difference between the calculated residual electrical battery voltage level and the predefined threshold value indicative of the minimum electrical battery voltage level needed for performing the auto injector process at the measured temperature is larger than the predetermined tolerance value, or
B) the calculated residual electrical battery voltage level is larger than the predefined threshold value at the measured temperature.

After the battery has been sufficiently re-charged, the first LED 1106 may stop flashing and a second LED may be turned on, e.g. emitting light having a different color than that of the first LED 1106, e.g. a green color compared to a red color of the first LED. The flashing of the second color is meant to indicate to the user, that the battery has been sufficiently recharged for performing the auto injector process at the temperature measured by the temperature sensor.

FIG. 2 shows an exemplary system 2 comprising an auto injector with a cartridge. The system 2 comprises the auto injector 4, as described in relation to FIG. 1, and an exemplary cartridge 700 received in the cartridge receiver 300 by front loading. The cartridge 700 is shown with a needle cover 908. The needle cover 908 extends out of the contact member 1102 to allow removal of the needle cover 908 from the cartridge 700.

By front loading is understood that at least the cartridge 700 is received with its needle end pointing out of the cartridge receiver opening 301. When the cartridge is being inserted and especially when it is fully inserted or almost fully inserted, the cartridge or cartridge assembly may be substantially covered by the housing or the contact member 1102. Especially in this situation the needle cover 908 serves as a protective means that makes it possible for a user at least to press on the needle cover 908 or a tip thereof to fully insert the cartridge without being injured by the needle. When the cartridge is fully inserted and sits in a retention position it is possible to detach the needle cover such that the auto injector is ready for use to inject the medicament or a portion thereof contained in the cartridge. After use, i.e. when a dose of medicament has been injected, the needle cover is attached such that the needle cover again serves as a protective means that makes it possible for a user at least to press on the needle cover 908 or a tip thereof to remove the cartridge without being injured by the needle.

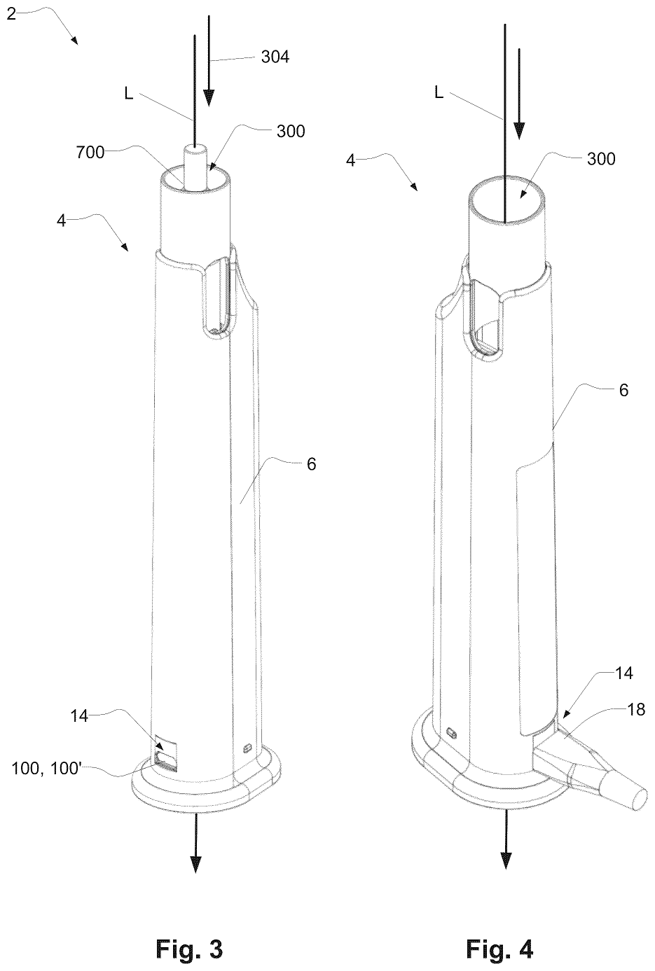

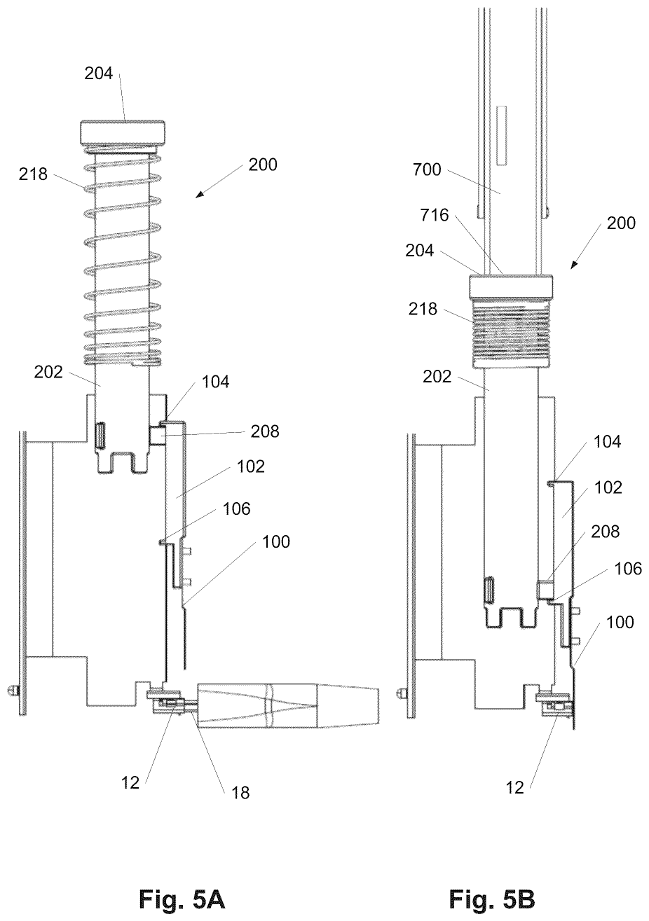

FIG. 3 shows the auto injector 4 with a cartridge, where the auto injector is turned 180 deg. compared to the view of the auto injector in FIGS. 1-2. The auto injector 4 comprises a first electrical connector 12 (see FIGS. 5A-B). The first electrical connector 12 is accessible via a connector opening 14 in the housing 6. The first electrical connector 12 accepts a second electrical connector 18 (see e.g. FIG. 4).

The connection of the second electrical connector 18 and the first electrical connector 12 may for example provide charging of a battery (not visible) of the auto injector 4. The battery may be accommodated by the housing 6. Alternatively or additionally, the connection of the second electrical connector 18 and the first electrical connector 12 may provide transferring of data to/from the auto injector 4, such as to/from a memory of the auto injector 4.

The auto injector 4 comprises a blocking member 100, 100′. The blocking member is configured to move between a blocking position and a non-blocking position. In the blocking position, the connector opening 14 is blocked, e.g. closed, as illustrated in FIG. 3. In the non-blocking position, the connector opening 14 is not blocked, e.g. open. In the non-blocking position a second electrical connector 18 (see e.g. FIG. 4) and the first electrical connector 12 may be connectable via the connector opening 14. In the blocking position, the blocking member 100, 100′ may prevent connection of a second electrical connector 18 and the first electrical connector 12.

The blocking member 100 may be movable along the longitudinal axis L, such as movable between the blocking position and the non-blocking position along the longitudinal axis L. For example, the blocking member 100 may be a sliding element, e.g. sliding along the longitudinal axis L.

Alternatively, the blocking member 100′ may be movable perpendicularly to the longitudinal axis L, such as movable between the blocking position and the non-blocking position perpendicular to the longitudinal axis L. For example, the blocking member 100′ may be a rotating element, e.g. rotating about the longitudinal axis L. The position of the blocking member 100, 100′ may be determined by insertion of a cartridge 700 in the cartridge receiver 300. The blocking member 100, 100′ may be in the blocking position when the cartridge 700 is received in the cartridge receiver 300, such as shown in FIG. 3. The blocking member 100, 100′ may be in the non-blocking position when the cartridge is not received in the cartridge receiver, such as shown in FIG. 4.

FIG. 4 shows an exemplary auto injector 4, as described in relation to previous figures, wherein a second electrical connector 18 is connected to the first electrical connector. The blocking member is in the non-blocking position to allow connection of the second electrical connector 18 to the first electrical connector 12 through the connector opening 14 of the housing 6.

The blocking member may be prevented to move to the blocking position. For example, the second electrical connector 18 may prevent the movement of the blocking member to the blocking position. For example, the second electrical connector 18 may obstruct the path of movement of the blocking member towards the blocking position.

Insertion of a cartridge in the cartridge receiver 300 may cause movement of the blocking member 100. For example, insertion of the cartridge in the cartridge receiver 300 may require movement of the blocking member to the blocking position. Thus, the blocking member being prevented from moving to the blocking position may prevent insertion of the cartridge. Thus, insertion of the cartridge in the cartridge receiver 300 may be prevented when the first electrical connector is connected to the second electrical connector 18.

FIG. 5A and FIG. 5B schematically illustrate selected parts of an exemplary auto injector as described in relation to previous figures.

FIG. 5A and FIG. 5B illustrate an ejector 200 of the auto injector. The ejector 200 comprises an ejector member 202. The ejector member 202 is movable along the longitudinal axis L. The ejector member 202 is movable between a first ejector position, shown in FIG. 5A, and a second ejector position, shown in FIG. 5B. The ejector member 202 is configured to follow movement of a cartridge 700 (only showed in part) when the cartridge 700 is received in the cartridge receiver 300 (see above figs.). As illustrated, when the cartridge 700 is received, the ejector member 202 is moved to the second ejector position. The ejector member 202 may be in the first ejector position when the cartridge 700 is not received in the cartridge receiver, as shown in FIG. 5A. The ejector member 202 may be in the second ejector position when the cartridge 700 is received in the cartridge receiver, as shown in FIG. 5B.

The ejector member 202 comprises an ejector abutment face 204. The ejector abutment face 204 is configured to abut a face, such as a cartridge back face 716, of the cartridge 700. By inserting the cartridge 700 into the cartridge receiver, the cartridge back face 716 may abut the ejector abutment face 204, and the ejector member 202 may be pushed towards the second ejector position.

The auto injector, such as the ejector 200 of the auto injector, comprises an ejector resilient member 218, such as a spring. The ejector resilient member 218 is configured to exert a force on the ejector member 202. For example, the ejector resilient member 218 may be configured to bias the ejector member 202 towards the first ejector position. For example, the ejector resilient member 218 may cause the ejector member 202 to be in the first ejector position, when a cartridge 700 is not received and/or being received in the cartridge receiver and/or being removed from the cartridge receiver. The ejector resilient member 218 may be compressed when the cartridge 700 is received in the cartridge receiver, as shown in FIG. 5B.

FIG. 5A and FIG. 5B illustrate a blocking member 100 of the auto injector. The ejector member 202 is coupled to the blocking member 100. The blocking member 100 comprises a first blocking coupling member 102. The ejector member comprises a second blocking coupling member 208. The first blocking coupling member 102 and the second blocking coupling member 208 are in engagement to translate movement of the ejector member 202 to the blocking member 100.

The blocking member 100 is in the blocking position when the ejector member is in the second ejector position, as shown in FIG. 5B. The blocking member 100 is in the non-blocking position when the ejector member 202 is in the first ejector position, as shown in FIG. 5A.

In the non-blocking position, a second electrical connector 18 can be connected to the first electrical connector 12, as shown in FIG. 5A. In the blocking position, the blocking member 100 is positioned in front of the first electrical connector 12. Thereby, the second electrical connector 18 cannot be connected to the first electrical connector 12, when the blocking member 100 is in the blocked position.

Conversely, as seen in FIG. 5A, the blocking member 100 is not able to move to the blocking position, due to the second electrical connector 18 being connected to the first electrical connector 12. Thus, the ejector member 202 may be prevented from moving to the second ejector position. Thus, insertion of the cartridge may be prevented when the second electrical connector 18 is connected to the first electrical connector 12.

The blocking member 100 comprises a first blocking member stop 104, and a second blocking member stop 106. The first blocking coupling member 102 is formed as a slot comprising the first blocking member stop 104 and the second blocking member stop 106.

The second blocking coupling member 208 may comprise a protrusion arranged to catch the first blocking member stop 104 by movement in one direction, and arranged to catch the second blocking member stop 106 by movement in another direction, e.g. along the longitudinal axis. For example, the second blocking coupling member 208 may catch the first blocking member stop 104, as shown in FIG. 5A, upon movement of the ejector member towards the first ejector position, such as upon removal of the cartridge 700 from the cartridge receiver. The second blocking coupling member 208 may catch the second blocking member stop 106, as shown in FIG. 5B, upon movement of the ejector member towards the second ejector position, such as upon insertion of the cartridge 700 in the cartridge receiver.

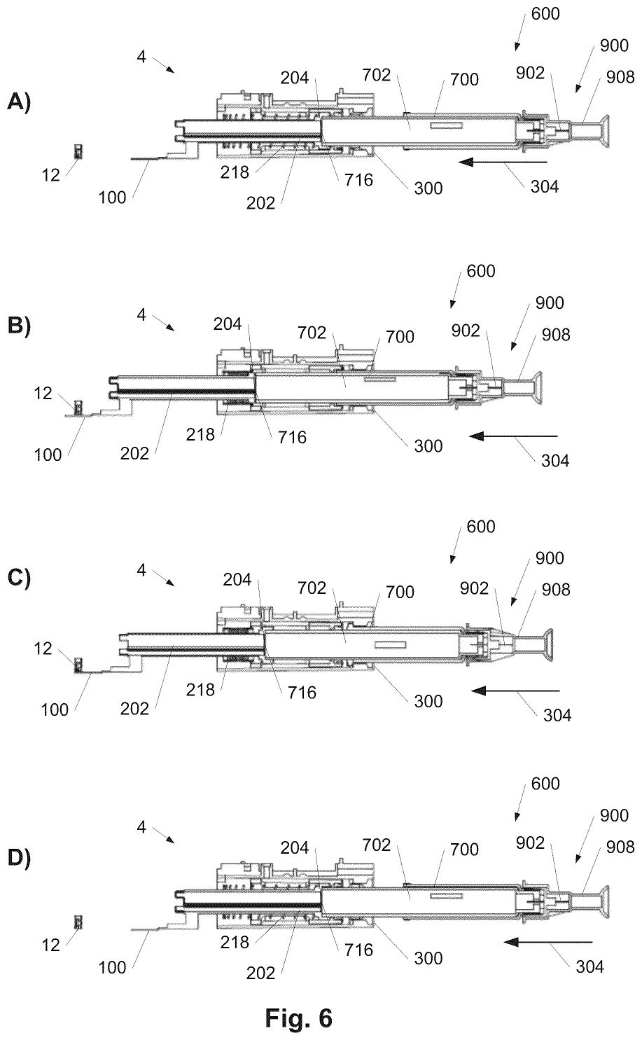

FIGS. 6A-6D schematically illustrate insertion and removal of an exemplary cartridge in an exemplary auto injector 4, such as the auto injector as described in relation to FIGS. 1-4. FIGS. 6A-6D only show selected parts of the exemplary auto injector 4.

The auto injector 4 comprises a first electrical connector 12, and a cartridge receiver 300 configured to receive a cartridge 700.

The auto injector 4 comprises an ejector member 202 and an ejector resilient member 218. The ejector member 202 comprises an ejector abutment face 204 configured to abut a face, such as a cartridge back face 716, of the cartridge 700. The auto injector further comprises a blocking member 100 coupled to the ejector member 202. In the example depicted, the ejector member 202 and the blocking member 100 are fixedly connected. The blocking member 100 is configured to block a connector opening to the first electrical connector 12, e.g. when the blocking member is in a blocked position.

Also illustrated in FIGS. 6A-6D is a cartridge assembly 600 comprising the cartridge 700. The cartridge 700 comprises a cartridge compartment 702. The cartridge compartment 702 may containing a medicament, or be configured to contain a medicament. The cartridge comprises a cartridge back face 716 configured to abut the ejector abutment face 204 of the ejector member 202.

The cartridge assembly 600 comprises a needle assembly 900. The needle assembly 900 comprises a needle 902, such as a hypodermic needle, and a needle cover 908. The needle cover 908 is covering the needle 902 such as to avoid contact with the needle 902. The needle cover 908 is removable. The needle cover 908 may be removed prior to initiating the injection of medicament.

FIG. 6A shows a first situation, wherein the cartridge 700 is about to be received in the cartridge receiver 300 in the cartridge receiving direction 304. The cartridge back face 716 has abutted the ejector abutment face 204. The ejector member 202 is in the first ejector position. The blocking member 100 is in the non-blocking position.

FIG. 6C shows a second situation following the first situation, wherein the cartridge 700 is moved to be received in the cartridge receiver 300. The cartridge 700 may be retained in the cartridge receiver 300 when received in the cartridge receiver 300. The cartridge receiver 300 is configured to selectively retain the cartridge 700 in the cartridge receiver 300. The ejector member 202 is in the second ejector position, and the blocking member 100 is in the blocking position. The ejector resilient member 218 is compressed. The cartridge 700 being retained in the cartridge receiver 300 prevents the ejector resilient member 218 from causing the ejector member 202 to move towards the first ejector position.

In case a second electrical connector had been connected to the first electrical connector 12, the blocking member 100 would be prevented from moving to the blocking position, and thus, the ejector member 202 would be prevented from moving to the second ejector position, since the ejector member 202 and the blocking member 100 are connected. Thus, the cartridge 700 would not be able to be received in the cartridge receiver 300, e.g. so as to be retained in the cartridge receiver 300, if the second electrical connector had been connected to the first electrical connector 12.

FIG. 6B shows an optional third situation between the first situation and the second situation, wherein the cartridge 700 is pushed further into the cartridge receiver 300 in the cartridge receiving direction 304. The ejector member is moved passed the second ejector position. The ejector resilient member 218 is compressed, and the blocking member 100 is moved passed the blocking position. This situation illustrates an example of how the cartridge receiver 300 may selectively retain the cartridge 700 in the cartridge receiver 300.

For example, the cartridge receiver 300 may retain the cartridge 700 following the cartridge 700 being pushed in the cartridge receiving direction causing movement of the ejector member 202 passed the second ejector position a first time. The cartridge receiver 300 may release the cartridge 700 following the cartridge 700 being pushed in the cartridge receiving direction and causing movement of the ejector member 202 passed the second ejector position a second time.

FIG. 6D shows a fourth situation, wherein the cartridge 700 is released from the cartridge receiver 300 and moved opposite the cartridge receiving direction 304 by the ejector resilient member 218 expanding. The ejector resilient member 218 causes the ejector member 202 to move towards the first ejector position. The retention member of the cartridge receiver 300 does not prevent movement of the cartridge 700, and the ejector resilient member 218 causes the ejector member 202 to move towards the first ejector position. By moving the ejector member 202 to the first ejector position, the blocking member 100 is moved to a non-blocking position. Thus, connection of a second electrical connector to the first electrical connector 12 is again possible.

Releasing the cartridge 700 from the cartridge receiver 300 may involve moving the cartridge in the cartridge receiving direction 304 as described in relation to FIG. 6B. Thus, the optional situation shown in FIG. 6B may optionally also be inserted between the situations of FIGS. 6C and 6D.

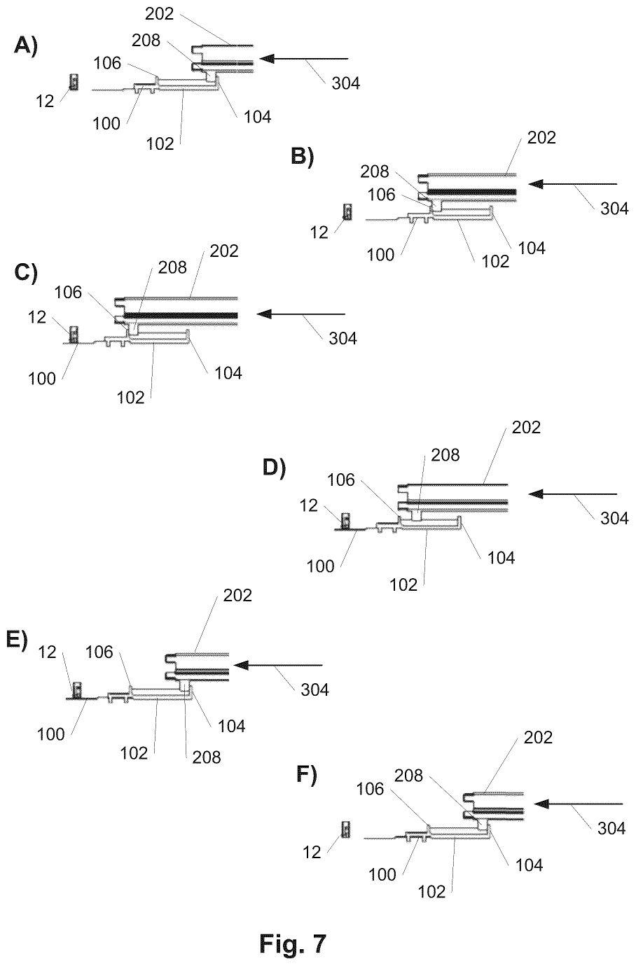

FIGS. 7A-7F schematically illustrate an exemplary coupling between a blocking member 100 and an ejector member 202. The blocking member 100 and ejector member 202 may be that of an exemplary auto injector, such as the auto injector as described in relation to FIGS. 1-4. FIGS. 7A-7F only show selected parts of the exemplary auto injector.

The auto injector, such as an ejector of the auto injector, comprises an ejector resilient member 218, such as a spring. The ejector resilient member 218 is configured to exert a force on the ejector member 202. For example, the ejector resilient member 218 may be configured to bias the ejector member 202 towards a first ejector position. The ejector member 202 may be movable between a first ejector position and a second ejector position. The first ejector position may be the position of the ejector member 202 when no cartridge is received in the cartridge receiver. The second ejector position may be the position of the ejector member 202 when a cartridge is received in the cartridge receiver. The ejector member 202 may be in other positions, such as a third ejector position and/or a fourth ejector position. The third ejector position and/or the fourth ejector position may be between the first ejector position and the second ejector position.

The blocking member 100 is configured to block a connector opening to the first electrical connector 12, e.g. when the blocking member 100 is in a blocked position.

The ejector member 202 is coupled to the blocking member 100. The blocking member 100 comprises a first blocking coupling member 102. The ejector member comprises a second blocking coupling member 208. The first blocking coupling member 102 and the second blocking coupling member 208 are in engagement to translate movement of the ejector member 202 to movement of the blocking member 100.

The blocking member 100 comprises a first blocking member stop 104, and a second blocking member stop 106. The first blocking coupling member 102 is formed as a slot comprising the first blocking member stop 104 and the second blocking member stop 106. The second blocking coupling member 208 is arranged to catch the second blocking member stop 106 by movement in one direction, e.g. in the cartridge receiving direction 304, and arranged to catch the first blocking member stop 104 by movement in an opposite direction, e.g. opposite the cartridge receiving direction 304.

FIG. 7A shows a first situation, e.g. when no cartridge is received in the cartridge receiver. The ejector member 202 is in the first ejector position, and the blocking member 100 is in the non-blocking position. Thus, a second electrical connector may be connected to the first electrical connector 12.

FIG. 7B shows a second situation, e.g. wherein a cartridge is being received in the cartridge receiver. The ejector member 202 is in the third ejector position. Compared to the previous figure, the ejector member 202 has moved in the cartridge receiving direction 304, e.g. caused by insertion of a cartridge in the cartridge receiver. The second blocking coupling member 208 abuts the second blocking member stop 106. Thus, from the third ejector position, movement of the ejector member 202 in the cartridge receiving direction 304 will result in movement of the blocking member 100 in the cartridge receiving direction 304.

FIG. 7C shows a third situation, e.g. wherein the cartridge has been further pushed in the cartridge receiving direction 304, e.g. for receiving the cartridge in the cartridge receiver. The ejector member 202 is in the second ejector position. The blocking member 100 is in the blocking position. Comparing with the previous figure, the ejector member 202 has moved, e.g. caused by the cartridge being received in the cartridge receiver, in the cartridge receiving direction 304. The second blocking coupling member 208 has moved with the ejector member 202, and by abutment with the second blocking member stop 106 the movement of the ejector member 202 to the second ejector position has caused the blocking member 100 to move to the blocking position.

FIG. 7D shows a fourth situation, wherein the ejector member 202 is in a position, wherein second coupling member 208 does not abut any of the first blocking member stop 104 or the second blocking member stop 106. For example, such a position may be between the second ejector position and the third ejector position and/or the fourth ejector position. For example, the ejector member 202 may be in such a position after the cartridge has been received in the cartridge receiver. In the illustrated situation, e.g. in the illustrated position of the ejector member 202, movement of the ejector member 202 does not immediately translate into movement of the blocking member. The engagement of the first blocking coupling member 102 and the second blocking coupling member 208 allows a distance of slack between movement of the ejector member 202 and the blocking member 100.

FIG. 7E shows a fifth situation, e.g. wherein the cartridge is being released from the cartridge receiver, thus being moved opposite the cartridge receiving direction 304. The ejector member 202 is in the fourth ejector position. The blocking member is in the blocking position. Compared to the previous figure, the ejector member 202 has moved opposite the cartridge receiving direction 304 to the fourth ejector position, e.g. caused by the ejector resilient member (see previous figs.). The second blocking coupling member 208 abuts the first blocking member stop 104. Thus, from the fourth ejector position, movement of the ejector member 202 opposite the cartridge receiving direction 304 will result in movement of the blocking member 100 opposite the cartridge receiving direction 304.

FIG. 7F shows a sixth situation, e.g. wherein the cartridge has been removed from the cartridge receiver. The ejector member 202 is in the first ejector position. The blocking member 100 is in the non-blocking position. Comparing with the previous figure, the ejector member 202 has moved, e.g. caused by the ejector resilient member (see previous figs.) and the cartridge being removed from the cartridge receiver. The second blocking coupling member 208 has moved with the ejector member 202, and by abutment with the first blocking member stop 104 the movement of the ejector member 202 to the first ejector position has caused the blocking member 100 to move to the non-blocking position.

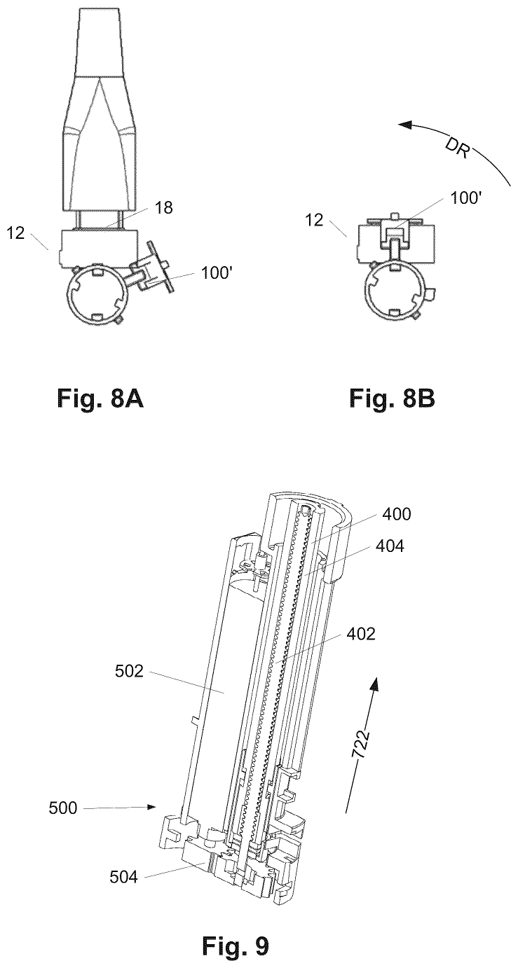

FIG. 8A and FIG. 8B show an exemplary blocking member 100′ of an exemplary auto injector, such as the auto injector of FIGS. 1-4. The blocking member 100′ as illustrated in FIG. 8A and FIG. 8B is a rotational blocking member. The blocking member 100′ is configured to rotate in a direction of rotation DR in response to translational movement of the ejector member in the cartridge receiving direction.

FIG. 8A shows the blocking member 100′ being in the non-blocking position. A second electrical connector 18 is connected to the first electrical connector 12.

FIG. 8B shows the blocking member 100′ being in the blocking position. Connection of a second electrical connector to the first electrical connector 12 is prevented by the blocking member 100′. Compared to FIG. 8A, the blocking member 100′ has been rotated in the direction of rotation DR, to the blocking position.

FIG. 9 schematically illustrates an exemplary drive module 500 and plunger rod 400. Such as a drive module 500 and a plunger rod 400 for an auto injector as described in relation to previous figures.

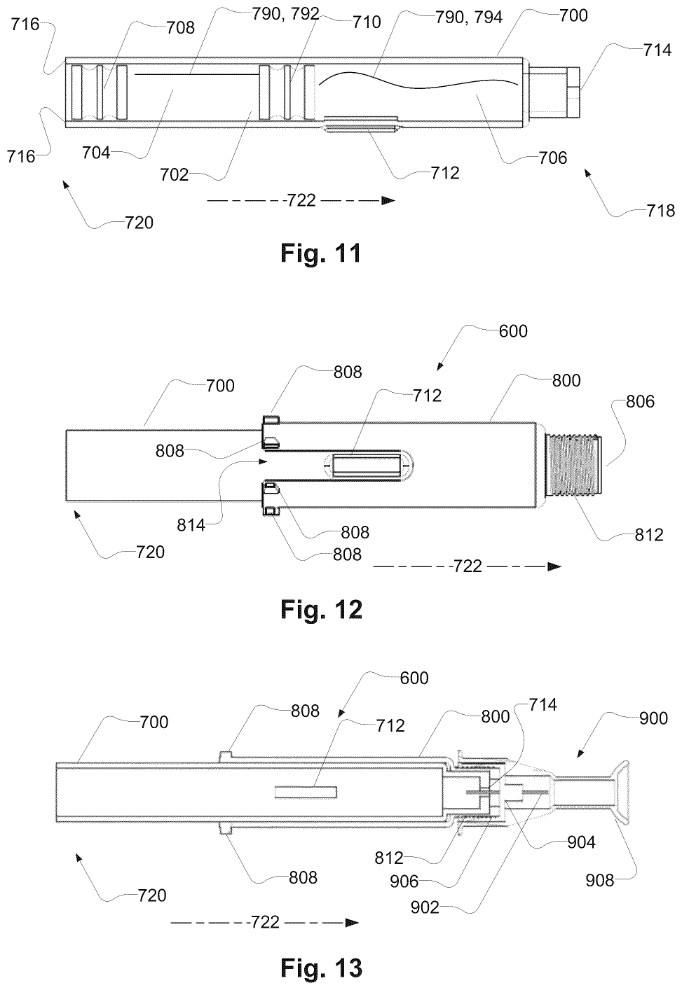

The plunger rod 400 is configured to advance a first stopper of a cartridge, such as a cartridge described in relation to FIG. 11, such as a cartridge received in the auto injector, such as received in the cartridge receiver of the auto injector. The plunger rod 400 comprises an outer plunger rod 404 with an inner thread, and an inner plunger rod 402 with an outer thread. The thread of the inner plunger rod 402 is in engagement with the thread of the outer plunger rod 404. The outer plunger rod 404 is prevented from rotating relative to the housing of the auto injector. The movement of the plunger rod 400 comprises rotation of the inner plunger rod 402. The rotation of the inner plunger rod 402 results in translational movement of the outer plunger rod 404, due to the outer plunger rod 404 being prevented from rotating. The outer plunger rod 404, when moved translationally in the first stopper direction 722, is configured to abut the first stopper of the cartridge, and to move the first stopper in the first stopper direction 722.

The drive module 500 is coupled to actuate the plunger rod 400. The drive module 500 is electrically connected to a battery for receiving electrical power. The drive module 500 comprises a motor 502, such as an electro-mechanical motor, such as a DC motor. The drive module 500 comprises a transmission 504 for coupling the motor 502 to the inner plunger rod 402 of the plunger rod 400.

Although the example shown comprises a motor 502, which may be an electro-mechanical motor, it will be readily understood that the auto injector 4 may be realised having an alternative drive module, such as comprising a solenoid motor, a shape memory metal engine, an arrangement of springs and/or a pressurized gas configured to actuate the plunger rod 400.

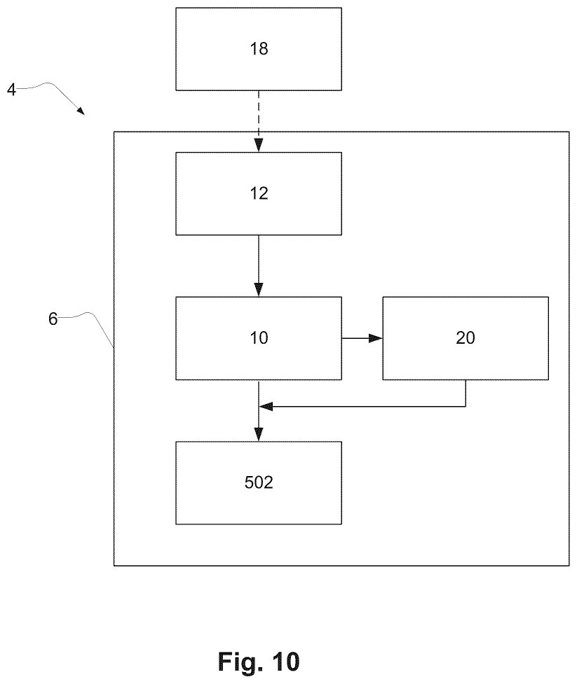

FIG. 10 schematically illustrates exemplary components of an exemplary auto injector 4, such as the auto injector 4 as described in relation to previous figures. A second electrical connector 18 may be connected to a first electrical connector 12. By doing so a battery 10 of the auto injector may be charged. The battery 10 may supply electrical power to a motor 502. A processing unit 20 may be electrically powered by electrical power from the battery 10. The processing unit 20 may control the flow of electrical power to the motor 502. For example, the processing unit 20 may control the motor 502 to turn on or off. The processing unit 20, the motor 502, the battery 10 and the first electrical connector 12 is accommodated in the housing 6 of the auto injector 4.

FIG. 11 schematically illustrates an exemplary cartridge 700, such as a cartridge 700 being configured to be received in the cartridge receiver of an auto injector, such as the auto injector described in relation to previous figures.

The cartridge 700 comprises a cartridge compartment 702. The cartridge compartment 702 may be configured for containing a medicament. The cartridge 700 has a first end 718 and a second end 720. The cartridge 700 comprises a cartridge outlet 714 at the first cartridge end 718. The cartridge may be configured to expel medicament through the cartridge outlet 714. The cartridge outlet 714 may be sealed by a needle penetrable sealing. The sealing may be made from rubber and optionally comprise a piercing which enables the needle to penetrate the sealing, while sealing the medicament when the needle is not penetrating the sealing.

The cartridge comprises a first stopper 708 movable inside the cartridge compartment, e.g. in a first stopper direction 722, e.g. towards the first cartridge end. For example, the medicament may be expelled through the cartridge outlet 714 upon movement of the first stopper 708 in the first stopper direction. The cartridge comprises a cartridge back face 716 at the second cartridge end. The cartridge back face 716 comprises a cartridge back end opening for providing access to the first stopper 708 for a plunger rod.

As illustrated, the cartridge 700 may be a dual chamber cartridge. The cartridge comprises a second stopper 710 movable inside the cartridge compartment 702, e.g. in the first stopper direction 722, e.g. towards the first cartridge end. The cartridge compartment 702 comprises a first cartridge sub-compartment 704 and a second cartridge sub-compartment 706. The first cartridge sub-compartment 704 is between the first stopper 708 and the second stopper 710. The second cartridge sub-compartment 706 is between the second stopper 710 and the cartridge outlet 714. The second cartridge sub-compartment 706 may comprise a medicament, such as a dry medicament, such as a medicament dried by lyophilization. The cartridge comprises a bypass section 712 for providing fluid communication between the first cartridge sub-compartment and the second cartridge sub-compartment. The bypass section 712 provides fluid communication between the first cartridge sub-compartment and the second cartridge sub-compartment when the second stopper 710 is positioned in the bypass section 712.

The first cartridge sub-compartment 704 contains a first medicament component 792 of the medicament 790. The first medicament component 792 may be a liquid as illustrated. The second cartridge sub-compartment 706 contains a second medicament component 794 of the medicament 790. The second medicament component 794 may be a dry composition. By positioning of the second stopper 710 within the bypass section 712, the first medicament component 792 may be transmitted into the second cartridge sub-compartment 706 via the bypass section 712, thereby mixing the first medicament component 792 and the second medicament component 794 to achieve the combined medicament 790.

The cartridge 700 may generally have a cylindrical form. However, the bypass section 712 may form a protrusion from the generally cylindrical form.

FIG. 12 shows a cartridge holder with a cartridge. The cartridge holder 800 accommodates at least a portion of the cartridge 700 by frictional coupling. Like the cartridge 700, the cartridge holder 800 may have a generally cylindrical form. An inner diameter of the cartridge holder 800 matches with an outer diameter of the cartridge. The cartridge holder may be made from a plastics material, whereas the cartridge typically is made from glass or a glass-like material; the cartridge may also be made from a plastics material.

As shown, the cartridge holder 800 accommodates the first cartridge end 718 of the cartridge. The cartridge holder 800 has a cartridge holder slot 814 which accommodates the bypass section 712 which is shown in FIG. 11 as a protruding member. The cartridge holder introduces coupling options to the cartridge in the form of a needle assembly coupling portion 812, which as shown may take the form of a threading, and cartridge retention members 808. The cartridge retention members 808 may take the form of protrusions that extends from the generally cylindrical form of the cartridge holder 800. The cartridge retention members 808 may be located at or close to the rim of the cartridge holder at the opposite end of the cartridge assembly outlet opening 806. In one or more examples, the cartridge retention members 808 are arranged at a greater distance from the rim than shown.

The needle assembly coupling portion 812 makes it possible to couple the cartridge 700, via the cartridge holder 800, to a needle assembly as will be described in greater detail below.

FIG. 13 shows a cross section of a cartridge assembly with a needle assembly. Note that the cartridge 700 is shown without stoppers, but with the bypass section 712.

The needle assembly 900 is coupled to the cartridge holder by a respective needle assembly coupling portion 812 and cartridge holder coupling portion 906 of the needle assembly and the cartridge holder. The coupling portions 812 and 906 may be in the form of threading.

The needle assembly 900 comprises a needle hub 904 that holds a needle 902. The needle hub 904 may have a bore at its one end into which the needle extends and on sidewalls of which the needle assembly coupling portion 812 is arranged. The needle assembly 900 also comprises a needle cover 908 that may be coupled by frictional coupling to the needle hub.

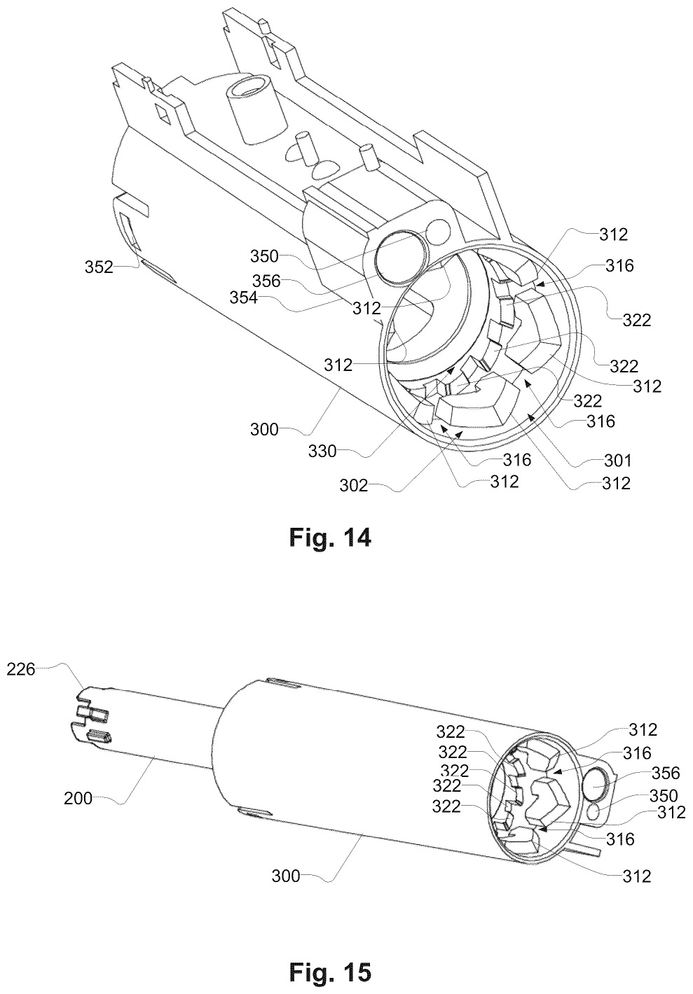

FIG. 14 shows a cartridge receiver. The cartridge receiver 300 has a cartridge receiver compartment 302 configured to receive a cartridge assembly 600 through a cartridge receiver opening 301. The cartridge receiver compartment 302 has a first section, at a first distance from the cartridge receiver opening 301, with inwardly extending first guide members 312 that are spaced apart to form passages 316 between the inwardly extending first guide members 312. The inwardly extending first guide members 312 form a first bore accepting the cartridge assembly 600 when inserted through the cartridge receiver opening 301. The diameter of the bore is slightly larger than an outer diameter of the cartridge assembly 600 excluding the cartridge retention members 808, but is smaller than the diameter of a circle circumscribing the cross section of the cartridge assembly 600 and the cartridge retention members 808. The cartridge retention members 808 can thereby not pass a first guide member 312 unless the cartridge is turned about the longitudinal axis, L, such that the cartridge retention members pass through the passages 316.

The cartridge receiver compartment 302 has an additional second section 330, with an annular shape, at a second distance, more remote than the first distance, from the cartridge receiver opening 301. The second section 330 has second guide members 322. The second guide members 322 are arranged with inclined faces and at angular positions about the longitudinal axis such that a cartridge retention member 808 having passed the passage 316 next to a first guide member 312 in the receiving direction is guided behind the first guide member 312 by an angular turning about the longitudinal axis, L.

The second guide members 322 form a second bore also accepting the cartridge assembly 600. The diameter of the second bore is substantially the same as the first bore, i.e. slightly larger than an outer diameter of the cartridge assembly 600 excluding the cartridge retention members 808, but smaller than the diameter of a circle circumscribing the cross section of the cartridge assembly 600 and the cartridge retention members 808. The cartridge retention members 808 can thereby not pass a second guide member 322. So when a cartridge retention member 808 lands on the second guide members 322, a turning of the cartridge assembly is inflicted.

The cartridge receiver 300 comprises flanges and coupling means such as opening 352 for coupling to other components of the auto injector (not shown). The cartridge receiver 300 also comprises a base 354 comprising a bore 356 for a spring (not shown) providing a spring-bias to the contact member 1102, which may be guided by a guide rod (not shown) accommodated in a bore 350.

FIG. 15 shows the cartridge receiver with an ejector. In this view the cartridge receiver 300 is shown in a different perspective than in FIG. 14. It is shown that an ejector 200 extends out of the cartridge receiver 300 from the other end of the cartridge receiver than the cartridge receiver opening end.

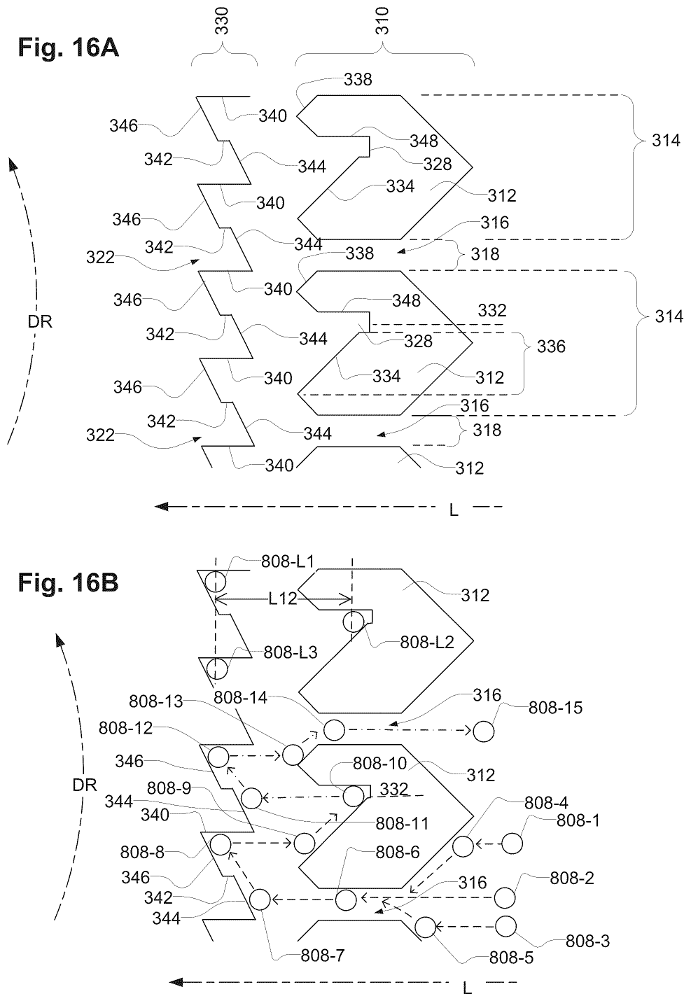

FIG. 16A shows a detailed view of the first section 310 and the second section 330 of the cartridge receiver compartment. In this view, portions of the first section 310 and the second section 330 are cut up along the longitudinal axis and fold out from their generally annular shape. The functional aspects of the first section 310 and the second section 330 are described in connection with FIG. 16B below. The dashed line at the bottom of FIG. 16A indicates the orientation of the longitudinal axis, L, and points in the receiving direction, which in this depiction is to the left. The cartridge receiver opening (not shown in this depiction) is located towards the right hand side. Thus, the first section 310 is arranged closer to the cartridge receiver opening than the second section 330. The curved dashed line to the left, DR, indicates a direction of rotation about the longitudinal axis when the first section 310 and the second section 330 are arranged as annular members.

The first section 310 comprises first guide members 312 and the second section 330 comprises second guide members 322.

The first guide members 312 extends over first guide member angles 314 and are spaced apart to form passages 316 at passage angles 318 between the first guide members 312.

The second guide members 322 have first faces 324 and second faces 326 (see e.g. FIG. 18). The first faces and the second faces are arranged alternately and separated by first riser portions 340. The first faces are, in the exemplary embodiment of FIG. 16A, divided into sections 344 and 346 separated by a second riser portion 342. Similarly, the second faces are, in the exemplary embodiment of FIG. 16A, divided into sections 344 and 346 separated by second riser portions 342. The first faces are inclined about a radial axis to the longitudinal axis and angularly arranged to extend at least partly over passage angles 318 and first guide member angles 314.

The first guide members 312 have a first guide face that faces the cartridge receiver opening, i.e. to the right hand side in this depiction, and forms a convex pointing shape with an apex directed towards the cartridge receiver opening.

The first guide members 312 also have a face that faces away from the cartridge receiver opening, i.e. to the left hand side in this depiction, and forms a concave shape with a slope portion 334, at slope angles 336, leading towards a retention portion 328 at or about a bottom portion of the concave shape at a retention angle 332. The slope portion 334 is inclined relative to the longitudinal axis and relative to the orthogonal thereto such that a turning of the cartridge is inflicted when a cartridge retention member 808 is pushed towards the slope portion 334 by a spring-loaded ejector 200. The turning brings the cartridge retention member 808 to the retention portion 328. To limit further turning of the cartridge assembly, a retention face 348 is provided substantially along the longitudinal axis.

Further, the first guide members 312 also have an eject face 338 with a slope that is inclined relative to the longitudinal axis and relative to the orthogonal thereto such that a turning of the cartridge is inflicted when a cartridge retention member 808 is pushed towards the slope portion 338 by a spring-loaded ejector 200.

FIG. 16B shows an inbound journey and an outbound journey of a cartridge retention member. The cartridge retention member 808 is depicted as an object with a circular cross section, e.g. in the form of a guide pin, but it may have other forms. The cartridge retention member 808 is shown at different positions indexed by a numeral following the reference numeral; for instance 808-1 indicates the position of the cartridge retention member 808 at position 1.

Dashed lines indicate a so-called inbound journey of the cartridge retention member 808 and dashed-dotted lines indicate a so-called outbound journey. To avoid cluttering the figure, not all reference numerals are inserted, however the reference numerals used in FIG. 16A apply to FIG. 16B for like-shaped elements.

When a cartridge assembly 600 with a cartridge retention member 808 is inserted, starting out in the receiving direction, the cartridge retention member 808 follows an inbound journey. As examples the inbound journey may start at position 1 or position 2 or position 3 at different angles. At position 2 the cartridge retention member 808 may pass straight into the passage 316 to position 6, whereas at position 1 the first guide member 312 inflicts a turning of the cartridge retention member, starting at position 4, where the cartridge retention member lands on the first guide member and continuing by the inflicted turning and longitudinal movement such that it is guided into the passage 316. Also at position 3 on the other side of the passage 316, a first guide member 312 inflicts a turning of the cartridge retention member, starting at position 5, where the cartridge retention member lands on the first guide member and continuing by the inflicted turning and longitudinal movement such that it is guided into the passage 316. Thus, substantially irrespective of the angle at which the cartridge retention member is received, it is guided into the passage 316.

Continuing its journey from a position, such as position 6, in the passage, in the receiving direction, the cartridge retention member lands on a second guide member 322 of the second section 330 and in particular on a first section 344 thereof. Due to the inclined face of the first section 344, a turning of the cartridge retention member 808 is inflicted, such that the cartridge retention member 808 turns from position 7 to position 8, where it meets one of the first riser portions 340 which prevents further rotation. At this position, a user inserting the cartridge assembly will feel that the cartridge assembly stops moving, and will therefore intuitively release the force used for insertion. At this position 8, a release of the force will make the spring-loaded ejector push the cartridge assembly and the cartridge retention member 808 outwards, opposite the receiving direction, to position 9. At position 9 the cartridge retention member 808 lands on the slope portion 334 of a first guide member 312 leading towards the retention portion 328 at or about a bottom portion of the concave shape at a retention angle 332. Due to the spring-loaded ejector working to push the cartridge retention member 808 outwards, the cartridge retention member 808 and hence the cartridge stays in a retention position, position 10.

It should be noted that as the cartridge retention member travels out over the second riser portion 342, it passes a point of no return and the inbound journey is generally not reversible. So, should the user ease the force used for insertion before position 8, but after the point of no return, the cartridge still ends up in the retention position.

While in the retention position the cartridge and the cartridge assembly may be prevented from moving in the receiving direction by a lock that introduces a stop. Thereby the cartridge stays in its retention position even if a force overcoming the spring-load on the cartridge or cartridge assembly is applied e.g. while the needle penetrates the skin. When the lock is released again to remove the stop, an outbound journey can start.

An outbound journey starts out from position 10 and is initiated when the spring-loaded force is overcome in the receiving direction, e.g. by a user pressing on a needle cover of the cartridge assembly. The cartridge retention member then lands on the second face 326 (see e.g. FIG. 18) and in particular a first section 344 thereof at position 808-11. Therefrom, it is brought to positon 12. At this position, a user ejecting the cartridge assembly will feel that the cartridge assembly stops moving, since the cartridge retention member 808 meets a first riser portion 340, and will therefore intuitively release the force used for ejecting the cartridge assembly. In releasing the force, the spring-loaded ejector will push the cartridge assembly and the cartridge retention member 808 outwards, opposite the receiving direction, to position 13, where the cartridge retention member 808 meets an eject face 338 that guides the cartridge retention member 808 towards a position 14 in the passage 316 and onwards to a position 15 where the cartridge assembly is fully ejected and can be handled as needed e.g. to remove the cartridge from the cartridge assembly and dispose the cartridge.

It is noted that the direction of rotation, DR, is defined by the direction of the slopes of the first face and second face since they define in which direction turning is inflicted.

With respect to the length of an ejector rod 202 and the length of ejector cogs 226 thereof, to be explained in greater detail further below, it is noted that the cartridge retention member 808 should be allowed to travel between a first and a second extreme positions, L1 and L2, spaced apart at a longitudinal distance L12. At the position L2, the cartridge retention member 808 is at its retention position, i.e. an advanced position towards the cartridge receiver opening. At the position L1, the cartridge retention member 808 is at a ‘deepest’ position, in the receiving direction, given by the second face 326 or section 346 thereof. Thus, the ejector should be able to travel the distance L12. In one or more examples, the position L3 may be located at a ‘deeper’ position than L1, in which case the ejector should allow the cartridge assembly retention member 808 to travel between L3 and L2.

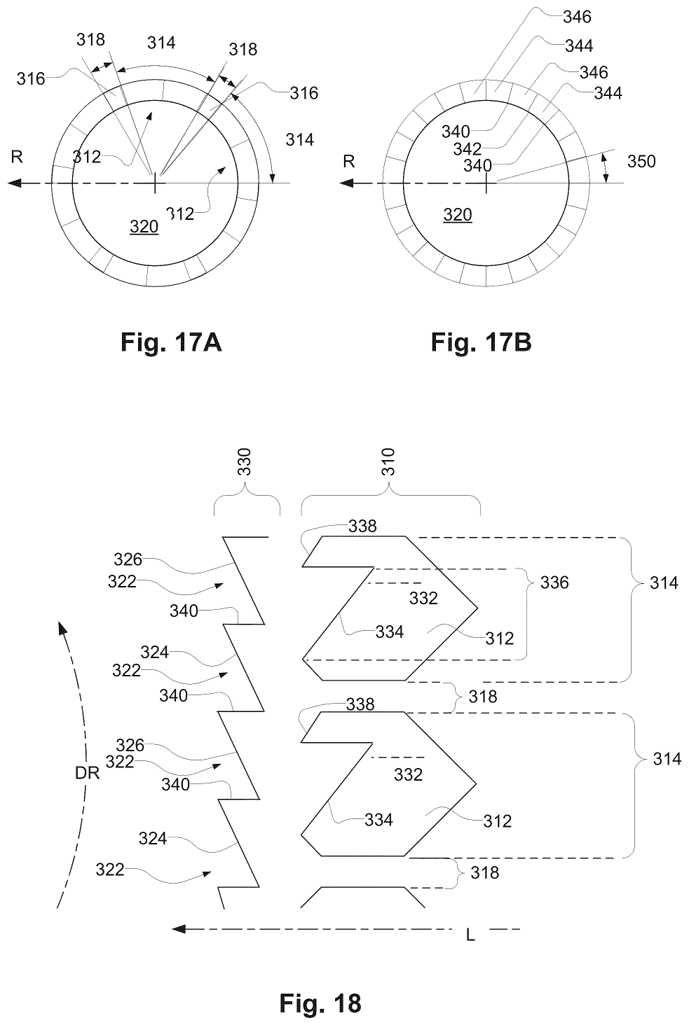

FIG. 17A is a cross-sectional view of the first section of the cartridge receiver compartment. This cross-sectional view is orthogonal to the longitudinal axis and shows first guide member angles 314 and passage angles 318 that extend over the first guide members 312 and the passage 316, respectively. The outwardly pointing apex of the first guide members is shown at a centre angle of the first guide member angles 314. The first bore is indicated by reference numeral 320.

The arrow designated by capital ‘R’ indicates a radial axis orthogonal to the longitudinal axis.

FIG. 17B is a cross-sectional view of the second section of the cartridge receiver compartment. This cross-sectional view is orthogonal to the longitudinal axis and shows the first sections 344 and the second sections 346 separated by first riser portions 340 and second riser portions 342.

Each of one of the first sections 344 and each of one of the second sections 346 may extend over section angles. Section angles may by e.g. about 15 degrees.

FIG. 18 shows a detailed view of alternative first section and second section of the cartridge receiver compartment. The first guide members 312 of the first section 310 have a concave shape with a slope portion 334 that extends across the retention angle 332.

The second guide members 322 of the second section 330 comprises a first face 324 that extends at least partly over passage angles 318 and first guide member angles 314. A second face 326 extends between first faces, alternately. The first riser portions 340 separate the first faces 324 and the second faces 326.

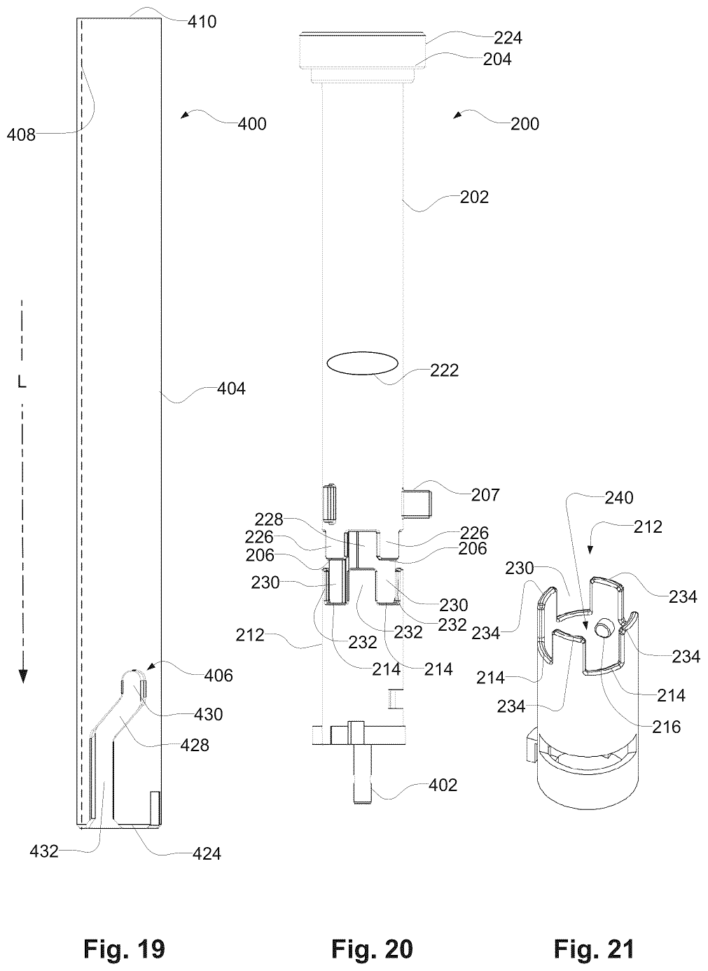

FIG. 19 shows an outer plunger rod 404. The outer plunger rod 404 is comprised by the plunger 400 and has a plunger rod front end 410 with a dimension that allows it to extend inside the cartridge to move a stopper therein. The outer plunger rod 404 may be moved by an inner plunger rod (not shown) and the inner plunger rod and the outer plunger rod may be coupled by threading such that turning of the inner plunger rod inflicts a longitudinal movement of the outer plunger rod 404. The outer plunger rod 404 may be retained at an angle about the longitudinal axis by means of a longitudinally extending plunger rod groove 408 in the outer wall of the outer plunger rod.

The outer plunger rod 404 is configured with a plunger rod track 406. The track 406 may extend from the rim of the outer plunger rod 404 at the other end than the plunger rod front end 410. The plunger rod track 406 has at least a first track portion 428 that leads an ejector lock guide pin 216 (see e.g. FIG. 21) from a first angle to a second angle that are angularly spaced apart to turn the ejector lock 212 (see e.g. FIG. 21) from the first angular position to the second angular position. A second track portion 432 extends from a plunger rod distal rim 424 along the longitudinal axis, L, towards and to connect with the first track portion 428 which is inclined with respect to the longitudinal axis e.g. at an angle of about 45°, e.g. about 30-45°, with respect to the longitudinal axis, L. Thereby the second track portion 432 accommodates the ejector lock guide pin 216 when the outer plunger rod 404 is in a forward position, towards the cartridge receiver opening 301, to expel a medicament by moving the first stopper 708 in the first stopper direction as mentioned above. When the second track portion 432 accommodates the ejector lock guide pin 216, the ejector lock 212 is angularly positioned to prevent the ejector rod 202 from moving backwards. A third track portion 430 connects with the first track portion 428 and continues along the longitudinal axis towards the plunger rod front end 410. Thereby the third track portion 430 accommodates the ejector lock guide pin 216 when the outer plunger rod 404 is in a backward position, opposite the cartridge receiver opening 301, whereat the outer plunger rod 404 is moved backwards, away from the first stopper 708. Thus, the longitudinal position of the outer plunger rod 404 has the dual function of:
engaging/disengaging with/from the first stopper 708 to expel medicament or being withdrawn from the first stopper 708 to forgo expel of medicament or removing the cartridge assembly 600; and
locking/unlocking the ejector rod 202 via rotation of the ejector lock 212.

This is explained in greater detail below.

Thus, the outer plunger rod part 404 is configured with a plunger rod track 406 that engages with the ejector lock guide pin 216 and extends from a plunger rod distal rim 424 towards plunger rod front end 410 and thus the cartridge receiver opening 301.

FIG. 20 shows an ejector and an ejector lock. The ejector is generally designated 200 and comprises an ejector rod 202. The ejector lock is configured to engage with the ejector rod by turning to thereby introduce a stop that prevents the ejector rod 202 from moving in the receiving direction.

The ejector rod 202 has an ejector collar 224 arranged about an ejector support face 204, which supports the cartridge at a cartridge back face 716, which may have the form of a rim. The ejector rod 202 has an ejector rod bore 222 to form a longitudinal passage all through the ejector rod 202. The ejector rod bore 222 allows the outer plunger rod 404 to move along the longitudinal axis.

The ejector rod 202 is configured with one or more ejector cut-outs 228 to form one or more ejector cogs 226 between the ejector cut-outs 228. Complementary therewith, the ejector lock 212 is configured with one or more ejector lock cogs 232 between one or more ejector lock cut-outs 230, respectively. The ejector lock 212 is supported e.g. in a bearing that allows the lock to turn or be turned, at least a fraction of a revolution, while preventing a longitudinal movement. The ejector lock 212 may have a flange or a recess that engages with a complementary recess or protrusion, respectively, to maintain the ejector lock 212 in a fixed longitudinal position while allowing it to be turned at least a fraction of a revolution. The ejector cut-outs 228 are also denoted ejector slots 228.

As shown, the ejector lock 212 has an angular position such that the ejector lock cogs 232 align with the ejector cut-outs 228. The ejector rod 202 can thus move in the receiving direction until an end portion 206 of the ejector cogs abuts a bottom portion 214, of the ejector lock cut-outs 230, since the ejector lock cogs 232 and the ejector cogs 226 have substantially the same length. Thus, a bottom portion 214 of the ejector lock cut-outs 230 abuts the end portion 206 of the ejector cogs 226. The end portion 206 of the ejector rod 202 is also denoted an ejector rest portion 206.

When the ejector lock 212 is turned such that ejector cogs 226 align with ejector lock cogs 232, a stop is introduced and the ejector rod 202 is prevented from moving in the receiving direction. The stop is introduced because, in that angular position of the ejector lock 212, the end portion 206 of the ejector rod 202 abuts the ejector lock support portion 234 of the ejector lock 212. The ejector lock support portion 234 of the ejector lock 212 is also denoted an ejector lock support portion 234. The ejector rod 202 may be prevented from turning, by means of an angle retaining guide 207 when it engages with an angle retaining slot 238 (see e.g. FIG. 18), wherein the angle retaining slot 238 is arranged on or in the cartridge receiver 300 or a member rigidly coupled to the cartridge receiver 300.

FIG. 21 shows an ejector lock. The ejector lock 212 is shown in greater detail here. It can be seen that the ejector lock 212 is configured with an ejector lock bore 240 accepting at least an end portion of the outer plunger rod 404. An ejector lock guide pin 216 sits in the ejector lock bore 240 and extends inwardly from a wall thereof.

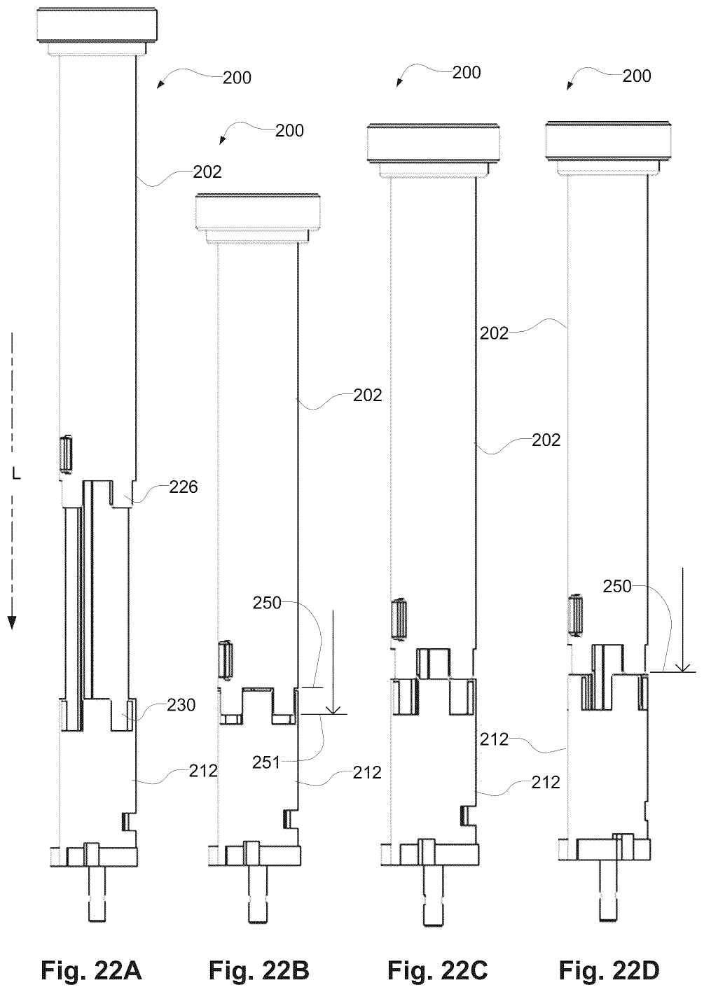

FIG. 22A through 22D shows various positions of the ejector relative to the ejector lock. In FIG. 22A the ejector rod 202 is shown in a longitudinal forward extreme position, relative to the ejector lock 212, where it is spring-biased to be when a cartridge assembly is not inserted through the cartridge receiver opening, e.g. as shown in FIG. 1. It can be seen that the ejector cogs 226 are aligned with ejector lock cut-outs 230, although displaced far from each other, such that the stop is disengaged and the ejector rod 202 is thus allowed to move to an extreme position in the receiving direction. Thereby the inbound journey and the outbound journey described in connection with FIG. 16B can take place during insertion or ejection of a cartridge assembly.

In FIG. 22B the ejector rod 202 is shown in a longitudinal backward extreme position e.g. when a cartridge assembly retention member 808 is in contact with the second section 330 of the retention mechanism. This position of the cartridge assembly retention member 808 corresponds to the position L1 or L3 shown in FIG. 16B.

In FIG. 22C the ejector rod 202 is shown in a longitudinal position where the cartridge assembly retention member 808 is in the retention position abutting the bottom of the concave shape of the first guide member 312 of the first section 310. This position of the cartridge assembly retention member 808 corresponds to the position L2 shown in FIG. 16B.

In FIG. 22D the ejector rod 202 is shown in the same longitudinal position as in FIG. 22C, but the ejector lock 212 is turned such that the stop is engaged. In this situation the ejector cogs 226 abuts the ejector lock cogs 232 end-to-end. Thus, an end portion 206 of the ejector cogs 226 abuts the end portion 234 of the ejector lock cogs 232.

Thus, at least both of the length of the ejector rod 202 and the length of the cogs and the cut-outs should be dimensioned to allow the cartridge assembly retention member 808 to travel between position L1 and L2 and L3 and L2.

Thus, the elongated ejector 200 is suspended to move along the longitudinal axis, L, and is enabled to move beyond a stop position 250 (see e.g. FIG. 22B) at a first angular position of ejector lock 212 and is prevented from moving beyond the stop position 250 by the ejector cogs 226 abutting the ejector lock cogs 232 at a second angular position of the ejector lock 212 (see e.g. FIG. 22D). At the first angular position of ejector lock, the elongated ejector is enabled to move beyond the stop position, such as further until an extreme position 251 since the ejector cogs enter the ejector lock cut-outs 230 between the ejector lock cogs 232. In one or more examples, the ejector rod is a substantially cylindrical rod coaxially arranged with the ejector lock, which comprises a substantially cylindrical portion.

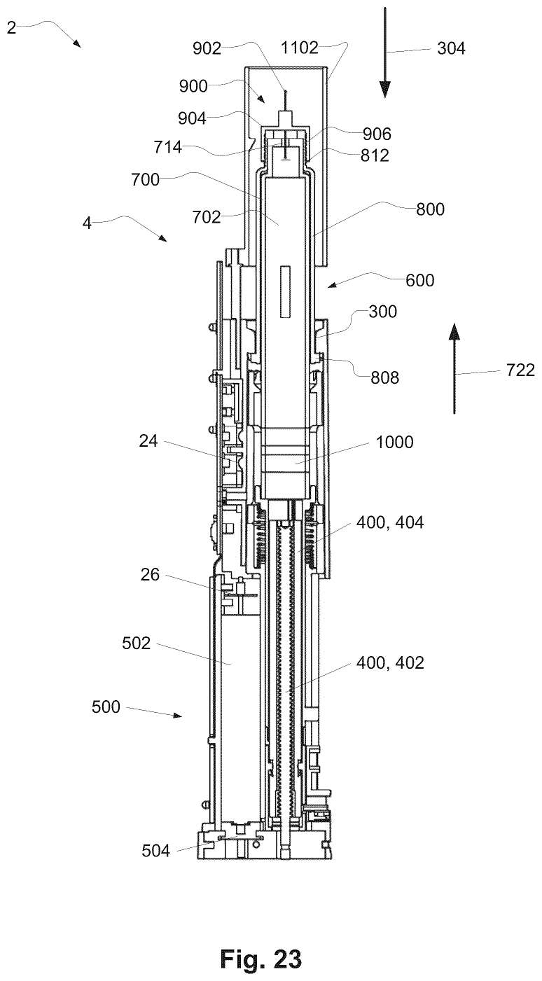

FIG. 23 shows a cross section of an exemplary system 2 comprising an auto injector 4, as described, for example, in relation to FIG. 1, and a cartridge assembly 600. The cartridge assembly 600 comprises a cartridge 700 with a cartridge compartment 702, a needle assembly 900, and a cartridge code feature 1000. The cartridge assembly 600 is received in the auto injector 4. The auto injector 4 comprises the ejector rod 202 as described above. The ejector rod 202 is suspended for longitudinal movement and is spring-loaded by an ejector spring 236 which spring-loads the ejector rod 202 in the direction opposite of the receiving direction. Thereby, during insertion of the cartridge assembly 600, the spring force exercised by the ejector spring 236 has to be overcome to insert the cartridge assembly 600 into a position where it is held in a retention position.

The cartridge assembly 600 comprises a cartridge holder 800. The cartridge holder 800 is configured for retention of the cartridge 700 in the cartridge receiver 300 of the auto injector 4. The cartridge holder 800 comprises a cartridge retention member 808. The cartridge retention member 808 engages with the cartridge receiver 300 for reception and retention of the cartridge 700 and the cartridge assembly 600 in the cartridge receiver 300.

The needle assembly 900 comprises a needle 902 and a needle hub 904. The needle assembly 900 is attached to the cartridge 700, e.g. by the needle hub 904 having a cartridge holder coupling portion 906, e.g. a threaded coupling portion, being in engagement with a needle assembly coupling portion 812 of the cartridge holder 800. The needle 902 extends through the cartridge outlet 714 of the cartridge 700. The cartridge outlet 714 may be blocked by a resilient sealing being penetrated by the needle 902, when the needle assembly 900 is attached to the cartridge 700.

The auto injector 4 comprises a code sensor 24 configured to read the cartridge code feature 1000. When the cartridge assembly 600 is inserted, as shown, the cartridge code feature 1000 is lined up with the code sensor 24.

The auto injector 4 comprises a plunger rod 400. The plunger rod 400 is configured to advance a first stopper of the cartridge 700. The plunger rod 400 comprises an outer plunger rod 404 with an inner thread, and an inner plunger rod 402 with an outer thread. The thread of the inner plunger rod 402 is in engagement with the thread of the outer plunger rod 404. The outer plunger rod 404 is prevented from rotating relative to the housing of the auto injector. The movement of the plunger rod 400 comprises rotation of the inner plunger rod 402. The rotation of the inner plunger rod 402 results in translational movement of the outer plunger rod 404, due to the outer plunger rod 404 being rotationally restricted. The outer plunger rod 404, when moved translationally in the first stopper direction 722, is configured to abut the first stopper of the cartridge 700, and to move the first stopper in the first stopper direction 722.

The drive module 500 is coupled to actuate the plunger rod 400. The drive module 500 is electrically connected to a battery for receiving electrical power. The drive module 500 comprises a motor 502, such as an electro-mechanical motor, such as a DC motor. The drive module 500 comprises a transmission 504 for coupling the motor 502 to the inner plunger rod 402 of the plunger rod 400.

Although the example shown comprises a motor 502, which may be an electro-mechanical motor, it will be readily understood that the auto injector 4 may be realised having an alternative drive module, such as comprising a solenoid motor, a shape memory metal engine, an arrangement of springs and/or a pressurized gas configured to actuate the plunger rod 400.

The auto injector 4 comprises an ejection sensor 26, such as a plunger rod position sensor. The ejection sensor 26 is configured to detect the position of the plunger rod 400. In the illustrated example, the ejection sensor 26 comprises a tachometer configured to count/detect the revolutions of the motor 502. Thus, the position of the plunger rod 400 may be determined based on the count of revolutions of the motor 502. The ejection sensor 26 may, based on the detection of the position of the plunger rod 400, detect the expelling of medicament and/or air in the cartridge compartment. The position of the plunger rod 400 may be indicative of the position of the first stopper of the cartridge 700, e.g. the most advanced position of the plunger rod 400 during reception of the cartridge 700 may be indicative of the position of the first stopper of the cartridge 700.

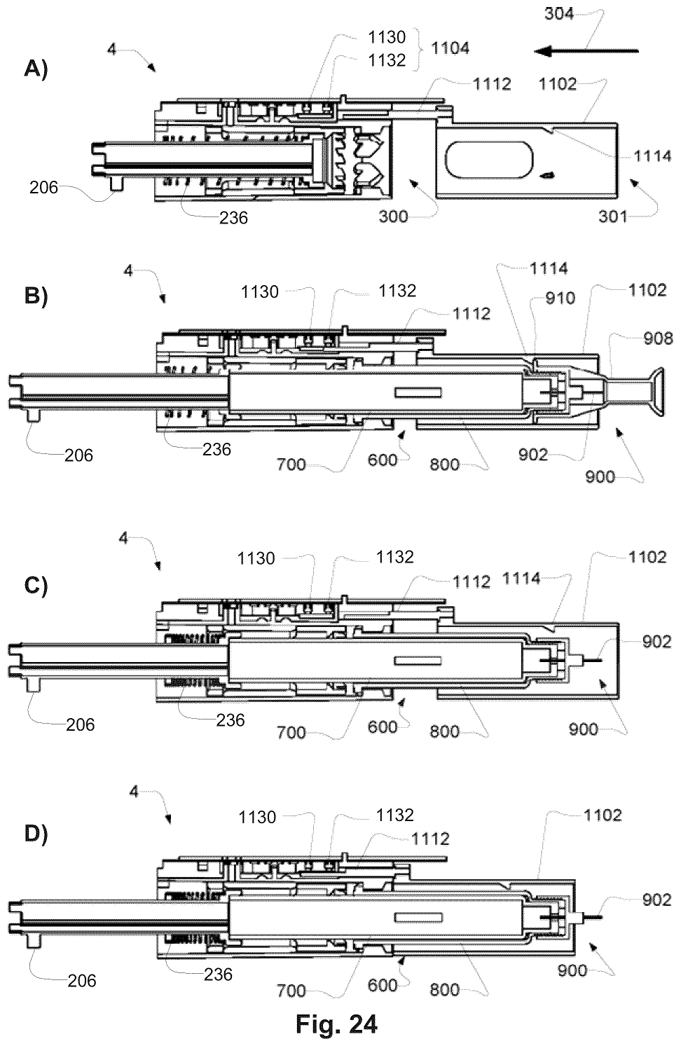

FIGS. 24A-D show cross sections of a portion of an exemplary system comprising an auto injector and a cartridge assembly. The auto injector 4 comprises a cartridge receiver 300 configured for receiving and retaining a cartridge. The auto injector 4 comprises a contact member 1102. The contact member 1102 may be movable between an extended contact member position and a retracted contact member position. The contact member 1102 comprises a contact member protruding part 1112. The contact member protruding part 1112 is configured to move with the contact member 1102. The contact member 1102 may be biased, e.g. by a contact member spring (not shown), towards the extended contact member position.

The contact member comprises a needle cover engagement member 1114. The needle cover engagement member 1114 is configured to abut a needle cover abutment face, e.g. of a needle cover positioned on the cartridge 700 inserted into the cartridge receiver 300.

The auto injector 4 comprises a contact member sensor 1104 configured to detect a position of the contact member 1102. The contact member sensor 1104 comprises a first contact member sensor 1130 and a second contact member sensor 1132. The first contact member sensor 1130 and the second contact member sensor 1132 may be optical sensors. The contact member sensor 1104 detects the position of the contact member 1102 by the contact member protruding part 1112 covering the first contact member sensor 1130 when the contact member 1102 is in a first contact member position, and the contact member protruding part 1112 covering the second contact member sensor 1132 when the contact member 1102 is in a second contact member position.

The first contact member position may be detected by the first contact member sensor 1130 being covered and the second contact member sensor 1132 being covered. The second contact member position may be detected by the first contact member sensor 1130 not being covered and the second contact member sensor 1132 being covered. The extended contact member position may be detected by the first contact member sensor 1130 not being covered and the second contact member sensor 1132 not being covered.

FIG. 24A schematically illustrates the auto injector 4 with no received cartridge and/or cartridge assembly. The contact member 1102 is in the extended contact member position. A cartridge may be inserted into the cartridge receiver 300 in the cartridge receiving direction 304 through the contact member 1102 defining a cartridge receiver opening 301.

FIG. 24B schematically illustrates the auto injector 4 with a cartridge assembly 600 received. The cartridge assembly comprises a cartridge 700, a cartridge holder 800 and a needle assembly 900. The needle assembly comprises a needle 902 and a needle cover 908. The needle cover 908 has a needle cover abutment face 910. The needle cover abutment face 910 engages the needle cover engagement member 1114 of the contact member 1102. The contact member 1102 is in the second contact member position, e.g. caused by the presence of the needle cover 908 and the abutment of the needle cover abutment face 910 on the needle cover engagement member 1114. The contact member protruding part 1112 covers the second contact member sensor 1132. The contact member protruding part 1112 does not cover the first contact member sensor 1130.

FIG. 24C schematically illustrates the auto injector 4 with a cartridge assembly 600 received. Compared to FIG. 24B, the needle cover 908 has been removed. The contact member 1102 is in the extended contact member position. The contact member 1102 is allowed to be moved to the extended contact member position since the needle cover abutment face 910 does not about the needle cover engagement member 1114. The contact member protruding part 1112 has moved with the contact member 1102. The contact member protruding part 1112 does not cover the second contact member sensor 1132. The contact member protruding part 1112 does not cover the first contact member sensor 1130.

FIG. 24D schematically illustrates the auto injector 4 with a cartridge assembly 600 received. The contact member 1102 is in the first contact member position. The first contact member position may be the retracted contact member position, or close to the retracted contact member position. The contact member 1102 may have been moved to the first contact member position by the contact member 1102 being pressed against an injection site, thereby inserting the needle 902 into the injection site. The contact member protruding part 1112 has moved with the contact member 1102. The contact member protruding part 1112 covers the first contact member sensor 1130. The contact member protruding part 1112 covers the second contact member sensor 1132.

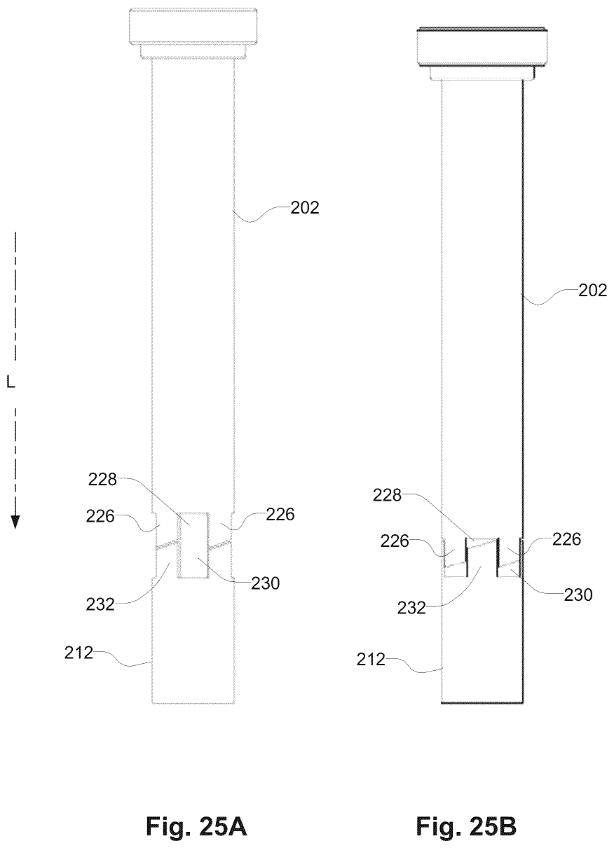

FIG. 25A-B shows various positions of the ejector relative to the ejector lock in an embodiment where the cogs have inclined faces. In FIG. 25A the ejector rod 202 and the ejector lock 212 are in a mutual position where the stop is engaged. However, it can be seen that the ejector lock cogs 232 and the ejector cogs 226 have inclined end portions that abut one another. Therefore, a further turning of the ejector lock 212 by a few degrees or a fraction of a degree may move the ejector 200 in the direction opposite of the receiving direction to displace it up to and tightly against the cartridge or cartridge assembly. Thereby, it is possible to reduce or eliminate clearances that would otherwise allow the cartridge to move slightly e.g. by as little as a fraction of a millimeter, but that could cause an inaccurate dose being expelled. The amount of tightening force may be controlled e.g. by monitoring current drawn by a motor coupled to drive the ejector lock.

In FIG. 25B the ejector 200 and the ejector lock 212 are in a mutual position where the stop is disengaged. Bottom portions of the ejector cut-outs 228 and/or the ejector lock cut-outs 230 may be inclined by the same angle as the end portions of the cogs to match the cogs or, as shown, have a substantially flat bottom portion, substantially orthogonal to the longitudinal axis.

According to a first item there is provided an auto injector (4) for administering injection of a medicament from a cartridge containing the medicament, the auto injector (4) comprising: a housing (6); a cartridge receiver (300) with a cartridge receiver compartment (302) configured to receive a cartridge assembly (600), with at least one cartridge retention member (808), when inserted through a cartridge receiver opening (301) along a longitudinal axis (L) in a receiving direction; wherein the cartridge receiver (300) has a passage (316) through which the at least one cartridge retention member (808) travels at least in the receiving direction, and a member (312) preventing movement beyond a retention position in a direction opposite of the cartridge receiving direction (304); an elongated ejector (200) that is configured with: an ejector support face (204) for supporting the cartridge or cartridge assembly (600) and an ejector rod (202) configured with one or more ejector cut-outs (228) to form one or more ejector cogs (226) between the ejector cut-outs (228); and an ejector lock (212) supported for turning at least a fraction of a revolution and maintained in a longitudinal position relative to the housing (6); wherein the ejector lock (212) is configured with one or more ejector lock cogs (232) between one or more ejector lock cut-outs (230); wherein the elongated ejector (200) is suspended to move along the longitudinal axis (L) and is enabled to move beyond a stop position (250) at a first angular position of ejector lock (212) and is prevented from moving beyond the stop position (250) by the ejector cogs (226) abutting the ejector lock cogs (232) at a second angular position of the ejector lock (212).

At the first angular position of ejector lock, the elongated ejector is enabled to move beyond the stop position since the ejector cogs enter the ejector lock cut-outs. In one or more examples, the ejector rod is a substantially cylindrical rod coaxially arranged with the ejector lock, which comprises a substantially cylindrical portion.

The ejector lock may be coupled directly or indirectly to drive means comprising a motor to rotate to and from the first angular position and the second angular position. In one or more examples, the ejector lock is rotated by manual operation.

In one or more examples, the ejector rod 202 has an ejector rod bore 222, forming a longitudinal passage through the ejector rod 202, wherein a plunger rod 400 is arranged to move longitudinally. The Plunger rod may comprise an outer plunger rod with an inner, longitudinal thread, and an inner plunger rod, such as a threaded rod, coupled such that rotation of the inner plunger rod causes the outer plunger rod to move longitudinally. The outer plunger rod may be prevented from rotating, while the inner plunger rod is supported to rotate and kept in a fixed longitudinal position. The inner plunger rod may be driven by a motor.

In one or more examples, the ejector lock is operated to rotate to and from the first angular position and the second angular position by being coupled to the plunger rod, whereby a longitudinal displacement of the plunger rod rotates the ejector lock.

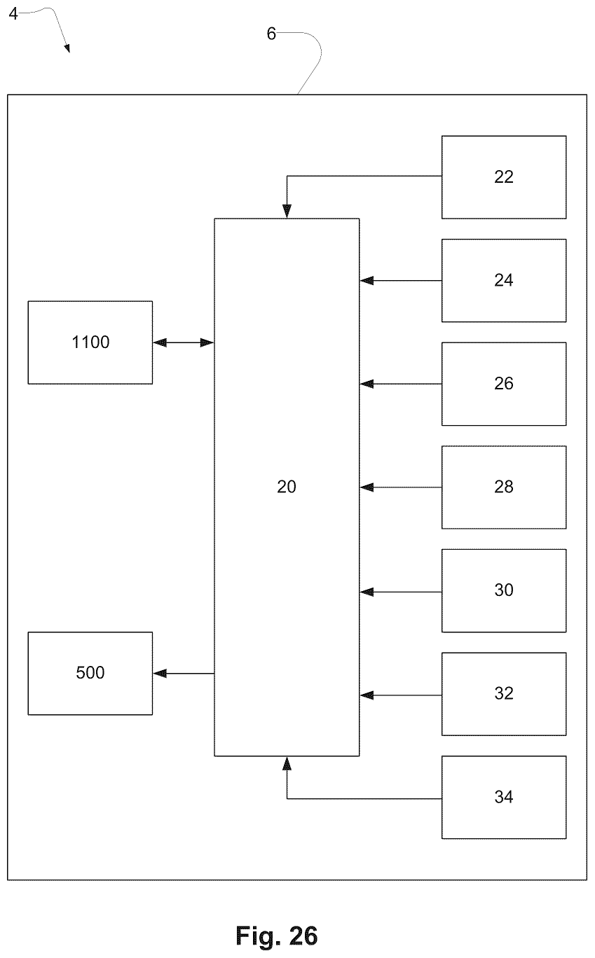

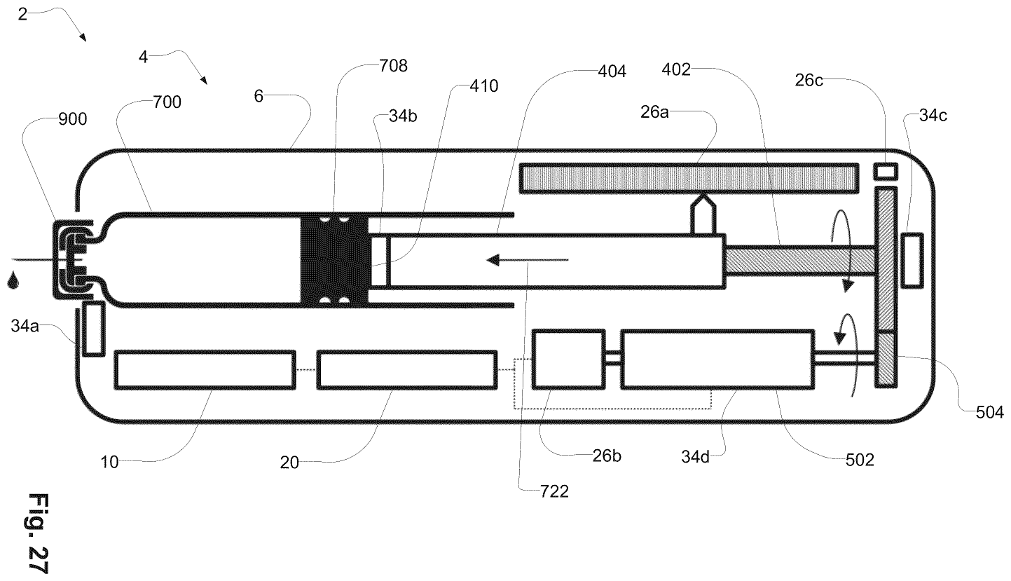

FIG. 26 shows a block diagram of an exemplary auto injector 4. The auto injector 4 comprises a plurality of sensors 22, 24, 26, 28, 30, 32, 34, a processing unit 20, a drive module 500, and a user interface 1100. The sensors 22, 24, 26, 28, 30, 32, 34 are coupled to the processing unit 20. The user interface 1100 is coupled to the processing unit 20. The processing unit is coupled to the drive module 500.

The processing unit 20 receives signals from the sensors 22, 24, 26, 28, 30, 32, 34 and the user interface 1100. The processing unit 20 is configured to control the drive module 500. The processing unit 20 may control the drive module 500 based on one or more of the received signals from the sensors 22, 24, 26, 28, 30, 32, 34 and the user interface 1100.

The auto injector 4 comprises an orientation sensor 22. The orientation sensor 22 is configured to provide an orientation signal indicative of the orientation of a cartridge received in the auto injector 4. For example, the orientation sensor 22 may be configured to detect the orientation of the auto injector 4. The orientation of the cartridge may be determined based on the orientation of the auto injector 4. The orientation sensor 22 may be configured to detect the direction of gravity. For example, the orientation sensor 22 may comprise an accelerometer.

The processing unit 20 is coupled to the orientation sensor 22. The processing unit 20 is configured to receive the orientation signal. The processing unit 20 may determine the orientation of the cartridge based on the orientation signal. The processing unit 20 may control the drive module 500 based on the orientation signal. For example, the processing unit 20 may be configured to control the drive module 500 to move a plunger rod based on the orientation signal. For example, the processing unit 20 may be configured to control the drive module 500 to move the plunger rod towards an extended plunger rod position only if the cartridge outlet is pointing upwards. Alternatively or additionally, the processing unit 20 may provide user output via the user interface 1100 based on the orientation signal.

The auto injector 4 comprises a code sensor 24. The code sensor 24 is configured to read a cartridge code feature. The code sensor 24 is configured to provide a code signal indicative of a cartridge code feature. For example, the code sensor may be configured to read/detect a color code.

The processing unit 20 is coupled to the code sensor 24. The processing unit 20 is configured to receive the code signal. The processing unit 20 may determine the cartridge code feature of the cartridge assembly based on the code signal. The processing unit 20 may be configured to determine a first plunger rod position and/or a second plunger rod position based on the code signal. The processing unit 20 may control the drive module 500 based on the code signal. For example, the processing unit 20 may be configured to control the drive module 500 to move the plunger rod towards the extended plunger rod position based on the code signal. Alternatively, or additionally, the processing unit 20 may provide user output via the user interface 1100 based on the code signal.

The auto injector 4 comprises a plunger rod position sensor 26. The plunger rod position sensor 26 is configured to detect the position of the plunger rod of the auto injector 4, and provide a plunger rod position sensor signal indicative of the position of the plunger rod. The plunger rod position sensor 26 may comprise a tachometer coupled to the drive module 500.

The processing unit 20 is coupled to the plunger rod position sensor 26. The processing unit 20 is configured to receive the plunger rod position sensor signal. The processing unit 20 may determine the position of the plunger rod based on the plunger rod position sensor signal. The processing unit 20 may control the drive module 500 based on the plunger rod position sensor signal. For example, the processing unit 20 may be configured to control the drive module 500 to start, stop or continue movement of the plunger rod based on the plunger rod position sensor signal. For example, the processing unit 20 may be configured to determine a plunger rod position based on the plunger rod position sensor signal. Alternatively or additionally, the processing unit 20 may provide user output via the user interface 1100 based on the plunger rod position sensor signal.

The processing unit 20 is coupled to the cartridge sensor 28. The processing unit 20 is configured to receive the cartridge sensor signal. The processing unit 20 may control the drive module 500 based on the cartridge sensor signal. For example, the processing unit 20 may be configured to control the drive module 500 to start movement of the plunger rod if a cartridge assembly is received, and/or only if a cartridge assembly is received. Alternatively or additionally, the processing unit 20 may provide user output via the user interface 1100 based on the cartridge sensor signal.

The code sensor 24 and the cartridge sensor 28 may be the same sensor, e.g. the code sensor 24 may be configured to detect reception of a cartridge assembly and subsequently read the cartridge code feature.

The auto injector 4 comprises a needle sensor 30. The needle sensor 30 is configured to detect a needle, and/or a needle assembly, and/or a needle cover of a needle assembly, of the cartridge assembly, when the cartridge assembly is received in the auto injector 4. The needle sensor 30 provides a needle signal indicative of the presence of a needle, and/or a needle assembly, and/or a needle cover of a needle assembly, of the cartridge assembly.

The processing unit 20 is coupled to the needle sensor 30. The processing unit 20 is configured to receive the needle signal. The processing unit 20 may control the drive module 500 based on the needle signal. For example, the processing unit 20 may be configured to control the drive module 500 to start movement of the plunger rod only if a needle is present, and/or only if a needle cover is not present, such as removed. Detection of a needle cover may be indicative of a needle being present. The processing unit 20 may be configured to control the drive module 500 to start only if a needle cover has been detected, and afterwards removed. Alternatively or additionally, the processing unit 20 may provide user output via the user interface 1100 based on the needle signal.

The auto injector 4 comprises a temperature sensor 32. The temperature sensor 32 is configured to detect a temperature, such as a temperature of the auto injector and/or of the cartridge and/or of the medicament. The temperature sensor 32 is configured to provide a temperature signal indicative of the temperature.

The processing unit 20 is coupled to the temperature sensor 32. The processing unit 20 is configured to receive the temperature signal. The processing unit 20 may be configured to determine the temperature, such as the temperature of the auto injector and/or of the cartridge and/or of the medicament based on the temperature signal. The processing unit 20 may control the drive module 500 based on the temperature signal. For example, the processing unit 20 may be configured to control the drive module 500 to move the plunger rod towards the extended plunger rod position based on the temperature signal. Alternatively or additionally, the processing unit 20 may provide user output via the user interface 1100 based on the temperature signal.

The auto injector 4 comprises a resistance sensor 34. The resistance sensor 34 is configured to detect resistance against movement of the plunger rod of the auto injector 4. The resistance sensor 34 may be configured to detect resistance against movement of the plunger rod based on measurements of the drive module 500. For example, the resistance sensor 34 may be configured to detect the electrical current of a motor of the drive module 500. For example, the resistance sensor 34 may be configured to determine the electrical current through the drive module. Alternatively or additionally, the resistance sensor 34 may be configured to measure pressure and/or force applied to a plunger rod front end of the plunger rod. The resistance sensor 34 is configured to provide a resistance signal indicative of resistance against movement of the plunger rod.

The processing unit 20 is coupled to the resistance sensor 34. The processing unit 20 is configured to receive the resistance signal. The processing unit 20 may be configured to determine the resistance against movement of the plunger rod based on the resistance signal. The processing unit 20 may control the drive module 500 based on the resistance signal. For example, the processing unit 20 may be configured to control the drive module 500 to adjust movement of the plunger rod based on the resistance signal. For example, the processing unit 20 may be configured to control the drive module 500 to start, stop or continue movement of the plunger rod based on the resistance signal.

Movement of the plunger rod results in a plunger rod speed. The processing unit 20 may be configured to determine the plunger rod speed. The processing unit 20 may be configured to control the drive module 500 to adjust, such as readjust, the movement of the plunger rod, if the resistance signal is indicative of resistance against movement of the plunger rod above a high resistance threshold. The processing unit 20 may further be configured to control the drive module 500 to adjust, such as readjust, the movement of the plunger rod, wherein adjusting the movement of the plunger rod may comprise increasing or decreasing the plunger rod speed. Alternatively or additionally, the processing unit 20 may provide user output via the user interface 1100 based on the resistance signal. The high resistance threshold may be based on the plunger rod position. The processing unit 20 may be configured to determine the high resistance threshold, e.g. based on the plunger rod position.

The processing unit 20 may be configured to determine the high resistance threshold based on the plunger rod position sensor signal, e.g. received from the plunger rod position sensor 26.

The auto injector 4 is illustrated comprising all of the above mentioned sensors. However, alternatively, the auto injector may comprise only one or any combination of one or more of the above mentioned sensors.

The auto injector comprises a user interface 1100. The user interface 1100 may comprise one or more input members, e.g. a first input member, for receiving a user input. The user interface is configured to provide a user input signal indicative of the received user input.

The processing unit 20 is coupled to the user interface 1100. The processing unit 20 is configured to receive the user input signal. The processing unit 20 may control the drive module 500 based on the user input signal. For example, the processing unit 20 may be configured to control the drive module 500 to move the plunger rod towards the extended plunger rod position based on the user input signal.

The auto injector comprises a housing 6 accommodating the sensors 22, 24, 26, 28, 30, 32, 34, processing unit 20, user interface 1100 and drive module 500.

FIG. 27 schematically illustrates a system 2 comprising an exemplary auto injector 4 with an inserted cartridge assembly comprising a cartridge 700 and a needle assembly 900. The auto injector 4 as shown in FIG. 27 illustrates different ways of implementing sensing of plunger rod position and resistance against movement of the plunger rod.

The plunger rod comprises an outer plunger rod 404 with an inner thread, and an inner plunger rod 402 with an outer thread. The thread of the inner plunger rod 402 is in engagement with the thread of the outer plunger rod 404. The outer plunger rod 404 is prevented from rotating relative to the housing 6 of the auto injector 4. Rotation of the inner plunger rod 402 results in translational movement of the outer plunger rod 404, due to the outer plunger rod 404 being rotationally restricted. The outer plunger rod 404, when moved translationally in the first stopper direction 722, is configured to abut the first stopper 708 of the cartridge 700, and to move the first stopper in the first stopper direction 722. The plunger rod front end 410 is configured to abut the first stopper 708.

A motor 502 is coupled to drive the plunger rod via a transmission 504. The motor 502 rotates a first part of the transmission 504, which rotates a second part of the transmission 504, which is coupled to rotate the inner plunger rod 402.

The motor 502 is controlled by a processing unit 20. The auto injector 4, such as the motor 502 and/or the processing unit 20, is powered by a battery 10, such as a rechargeable battery.

Position of the plunger rod, such as the position of the outer plunger rod 404 and/or the position of the plunger rod front end 410, may be determined by one or more position sensors 26a, 26b, 26c. For example, as illustrated, the plunger rod position may be determined by a position sensor 26a configured to sense position through a linear sensor coupled to the plunger rod, such as the outer plunger rod 404. Alternatively or additionally, as also illustrated, the plunger rod position may be determined by a position sensor 26b, such as a tachometer, configured to count/detect the revolutions of the motor 502. Alternatively or additionally, as also illustrated, the plunger rod position may be determined by a position sensor 26c, such as a tachometer, configured to count/detect the revolutions of the transmission 504 and/or a part of the transmission 504.

Resistance against movement of the plunger rod may be determined by one or more resistance sensors 34a, 34b, 34c, 34d. For example, as illustrated the resistance against movement of the plunger rod may be determined by a resistance sensor, such as a force sensor, 34a positioned in front of the cartridge 700, when the plunger rod advances the first stopper 708, the cartridge will press against the sensor 34a. Alternatively or additionally, as also illustrated, the resistance against movement of the plunger rod may be determined by a resistance sensor, such as a force sensor, 34b positioned on the plunger rod front end 410. Alternatively or additionally, as also illustrated, the resistance against movement of the plunger rod may be determined by a resistance sensor, such as a force sensor, 34c positioned to sense the reaction force from the plunger rod on first stopper 708, e.g. the sensor 34c may be positioned behind the inner plunger rod 402. Alternatively or additionally, as also illustrated, the resistance against movement of the plunger rod may be determined by a resistance sensor 34d configured to measure/detect the amount of current and/or power drawn by the motor 502.

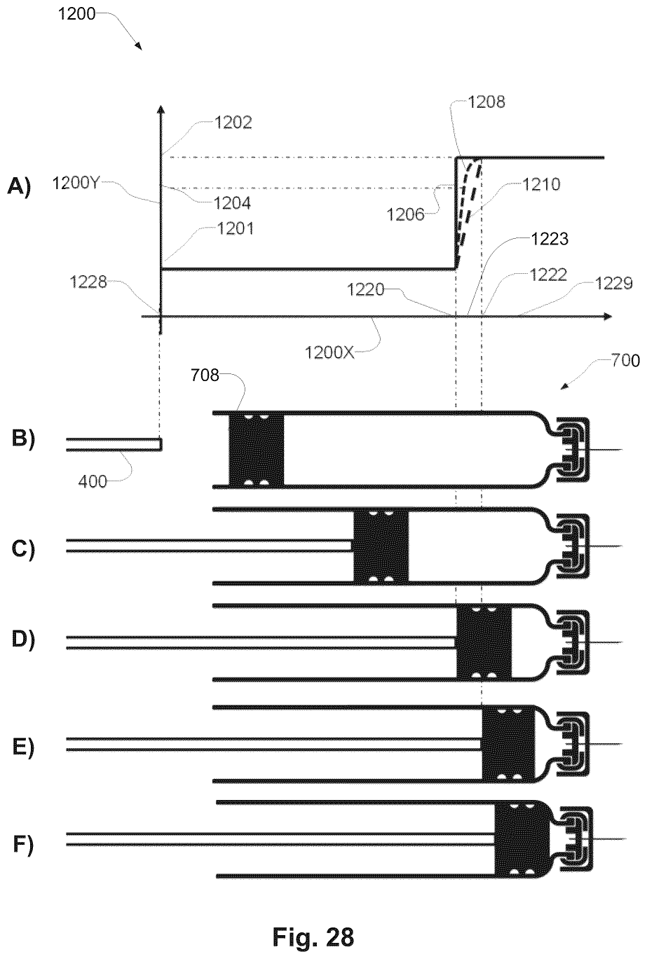

FIG. 28A shows a resistance graph 1200 illustrating a high resistance threshold depending on stopper position/plunger rod position, such as the high resistance threshold and the plunger rod position as described in relation to previous figures, and/or the stopper position associated with the plunger rod position as described in relation to previous figures. A plunger rod 400 is configured to move the first stopper 708, thus the position of the first stopper 708 is determined by the position of the plunger rod 400. Therefore, the position of the first stopper 708 may be corresponding to a position of the plunger rod 400. The plunger rod position may designate a plunger rod front end, such as the part of the plunger rod making contact with the first stopper 708.

The resistance graph 1200 has a first axis 1200X indicating stopper position/plunger rod position and a second axis 1200Y indicating resistance. Solid and dashed lines illustrate different examples of how the high resistance threshold may vary depending on stopper position/plunger rod position.

FIGS. 28B-F illustrates a plunger rod 400 and a cartridge 700 with a first stopper 708 in situations of corresponding exemplary plunger rod positions explained in the following. FIG. 28B shows the plunger rod 400 being in a retracted plunger rod position 1228. FIG. 28C shows the plunger rod 400 being in a position between the retracted plunger rod position 1228 and a first plunger rod position 1220. The first stopper 708 has been moved accordingly. FIG. 28D shows the plunger rod 400 being in the first plunger rod position 1220. The first stopper 708 has been moved accordingly to a first stopper position. FIG. 28E shows the plunger rod 400 being in a second plunger rod position 1222. The first stopper 708 has been moved accordingly to a second stopper position. FIG. 28F shows the plunger rod 400 being in a position between the second plunger rod position 1222 and an extended plunger rod position 1229. The first stopper 708 has been moved accordingly. The plunger rod position illustrated in FIG. 28F may be the extended plunger rod position 1229.

As illustrated by the graph in FIG. 28A, the high resistance threshold may be a first high resistance threshold 1201 when the plunger rod position is between the retracted plunger rod position 1228 and the first plunger rod position 1220. The high resistance threshold may be a second high resistance threshold 1202 when the plunger rod position is between the second plunger rod position 1222 and the extended plunger rod position 1229.

The second high resistance threshold 1202 may be higher than the first high resistance threshold 1201. For example, the first high resistance threshold 1201 may between 50-80 N, such as 50 N, 55 N, 60 N, 65 N, 70 N, 75 N, or 80 N. For example, the second high resistance threshold 1202 may be between 70-100 N, such as between 75-85 N, or such as between 80-90 N, or such as 70 N, 75 N, 80 N, 85 N, or 90 N. In an example, the first high resistance threshold 1201 500 N, and the second high resistance threshold 1202 is 80 N.

As illustrated by the solid line, the high resistance threshold may be the second high resistance threshold 1202 when the plunger rod position is between the first plunger rod position 1220 and the extended plunger rod position 1229. Alternatively or additionally, the high resistance threshold may be a third high resistance threshold 1204 when the plunger rod position is between the first plunger rod position 1220 and the second plunger rod position 1222, such as when the plunger rod position is at a third plunger rod position 1223. The third high resistance threshold 1204 may be higher than the first high resistance threshold 1201. The third high resistance threshold 1204 may be lower than the second high resistance threshold 1202.

The high resistance threshold may be increasing as a function of the plunger rod position. For example, as illustrated, the high resistance threshold may be increasing as the plunger rod is moved from the first plunger rod position 1220 to the second plunger rod position 1222. The solid and dashed lines illustrate exemplary ways the high resistance threshold may increase as the plunger rod is moved from the first plunger rod position 1220 to the second plunger rod position 1222. A first slope 1206 illustrates a step-change increase. A second slope 1208 illustrates a non-linear increase. A third slope 1210 illustrates a linear increase.

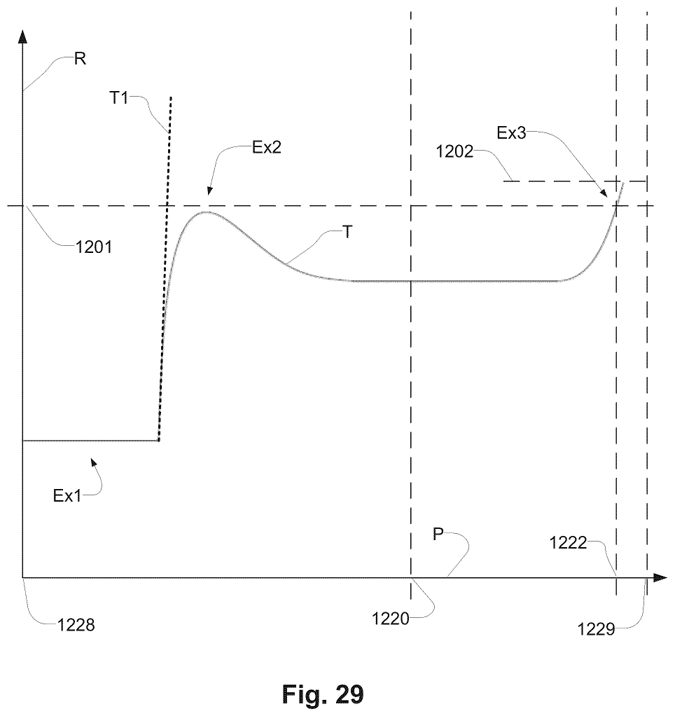

FIG. 29 shows an exemplary trace T of resistance R against movement of the plunger rod dependent on the position of the plunger rod P. The plunger rod is moved from a retracted position 1228 to an extended position 1229. In the beginning of the movement, the resistance against movement of the plunger rod is constant Ex1, e.g. the plunger rod does not yet push a stopper. Afterwards, a plunger rod front end of the plunger rod abuts a first stopper of the cartridge, and the resistance against movement of the plunger rod increases Ex2. The increased resistance is caused by the resistance against movement of the first stopper, e.g. due to frictional force. The resistance may decrease slightly after the first stopper has started moving, as illustrated. When the plunger rod approaches the extended plunger rod position 1229, the resistance may increase again Ex3, e.g. due to the first stopper approaching an end of the cartridge.

The trace T is an example of resistance against plunger rod movement when the cartridge received is a new and/or unused and/or normal cartridge. Other situations, such as situations wherein the cartridge received is apparently flawed, are exemplified by additional exemplary trace, T1.

Trace T1 illustrates an exemplary situation wherein the resistance against movement increases above a first high resistance threshold 1201, e.g. before the plunger rod position has passed the first plunger rod position 1220. Such situation may for example indicate that the first stopper is blocked from moving, e.g. the cartridge may be flawed. Following such situation, the plunger rod may be retracted to the retracted position and an error message may be provided through a user interface.

At a certain plunger rod position, such as the first plunger rod position 1220, the high resistance threshold may be changed, e.g. in order to allow for a higher resistance before aborting the movement of the plunger rod. As illustrated, at the end of the forward movement of the plunger rod, the resistance R increases, e.g. at the second plunger rod position 1222, to a resistance above the first high resistance threshold 1201. However, since the high resistance threshold at the second plunger rod position is a second high resistance threshold 1202, the movement of the plunger rod is continued. Eventually, as illustrated, the resistance against movement may reach the second high resistance threshold 1202, e.g. between the second plunger rod position and the extended plunger rod position 1229, and the movement of the plunger rod may be stopped.

The thresholds, such as the first high resistance threshold 1201 and/or the second high resistance threshold 1202 may be individually determined for the cartridge received. For example, a processing unit may be configured to determine one or more of the thresholds, based on a cartridge code feature of the cartridge and/or cartridge assembly received.

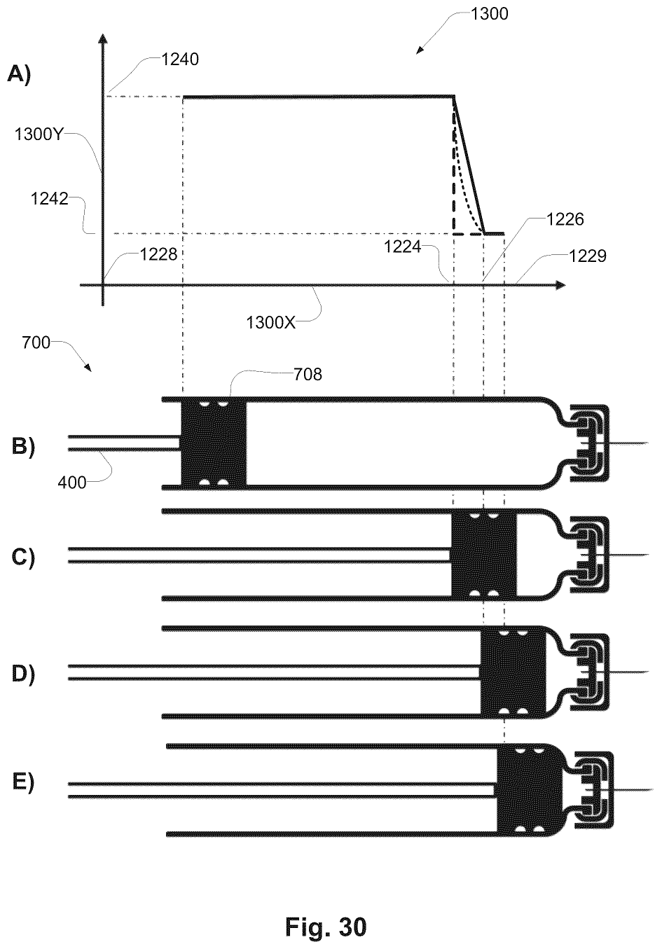

FIG. 30A shows a speed graph 1300 illustrating a plunger rod speed depending on stopper position/plunger rod position, such as the plunger rod speed and the plunger rod position as described in relation to previous figures, and/or the stopper position associated with the plunger rod position as described in relation to previous figures. A plunger rod 400 is configured to move the first stopper 708, thus the position of the first stopper is determined by the position of the plunger rod 400. Therefore, the position of the first stopper may be corresponding to a position of the plunger rod 400. The plunger rod position may designate a plunger rod front end, such as the part of the plunger rod making contact with the first stopper 708.

The speed graph 1300 has a first axis 1300X indicating stopper position/plunger rod position and a second axis 1300Y indicating speed, such as plunger rod speed. Solid and dashed lines illustrate different examples of how plunger rod speed may vary depending on stopper position/plunger rod position.

FIGS. 30B-E illustrate a plunger rod 400 and a cartridge 700 with a first stopper 708 in situations of corresponding exemplary plunger rod positions explained in the following. FIG. 30B shows the plunger rod 400 being in a position between a retracted plunger rod position 1228 and a fourth plunger rod position 1224. FIG. 30C shows the plunger rod 400 being in the fourth plunger rod position 1224. The first stopper 708 has been moved accordingly to a fourth stopper position. FIG. 30D shows the plunger rod 400 being in a fifth plunger rod position 1226. The first stopper 708 has been moved accordingly to a fifth stopper position. FIG. 30E shows the plunger rod 400 being in a position between the fifth plunger rod position 1226 and an extended plunger rod position 1229. The first stopper 708 has been moved accordingly. The plunger rod position illustrated in FIG. 30E may be the extended plunger rod position 1229.

As illustrated by the graph in FIG. 30A, the plunger rod speed may be based on the plunger rod position. For example, the plunger rod speed may be a first plunger rod speed 1240 when the plunger rod position is between the retracted plunger rod position 1228 and the fourth plunger rod position 1224. The plunger rod speed may be a second plunger rod speed 1242 when the plunger rod position is between the fifth plunger rod position 1226 and the extended plunger rod position 1229. The second plunger rod speed 1242 may be lower than the first plunger rod speed 1240. Alternatively, the second plunger rod speed 1242 may be higher than the first plunger rod speed 1240 in order to effectively empty the cartridge.

A plunger rod position may coincide with another plunger rod position. For example, the fourth plunger rod position 1224 may be the first plunger rod position 1220 as mentioned in relation to FIG. 28. The fifth plunger rod position 1226 may be the second plunger rod position 1222 as mentioned in relation to FIG. 28.

The plunger rod speed may be decreasing as a function of the plunger rod position. For example, the plunger rod speed may be decreasing as the plunger rod is moved from the fourth plunger rod position 1224 to the fifth plunger rod position 1226. The solid line illustrates an exemplary linear decrease of the plunger rod speed as the plunger rod is moved from the fourth plunger rod position 1224 to the fifth plunger rod position 1226. Other examples may be non-linear decrease and step-change decrease as exemplified by the dashed lines.

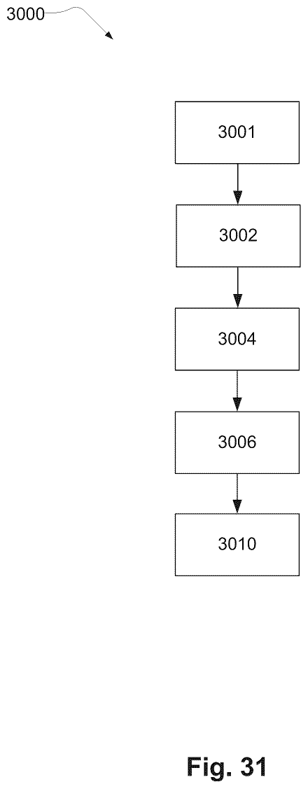

FIG. 31 shows a flow chart of an exemplary method 3000 for operating and/or controlling an auto injector, such as the auto injector as described in relation to previous figures.

The method 3000 comprises receiving 3001 a cartridge comprising a first stopper; moving 3002 a plunger rod towards an extended plunger rod position; determining 3004 plunger rod position; receiving a resistance signal 3006; and adjusting 3010 the movement of the plunger rod.

Receiving 3001 the cartridge may comprise receiving the cartridge in a cartridge receiver of the auto injector.

Moving 3002 the plunger rod may comprise moving the plunger rod from a retracted plunger rod position. Moving 3002 the plunger rod may comprise moving the plunger rod in a first plunger rod direction.

Determining 3004 plunger rod position may be determined by a processing unit of the auto injector. Determining 3004 plunger rod position may be based on detection from a sensor, such as a plunger rod position sensor, e.g. comprising a tachometer.

Receiving a resistance signal 3006 may comprise receiving the resistance signal from a resistance sensor. The resistance signal may be indicative of resistance against movement of the plunger rod, such as movement towards the extended plunger rod position, such as movement in the first plunger rod direction.

Adjusting 3010 the movement may comprise stopping the movement of the plunger rod. Alternatively or additionally, adjusting 3010 the movement may comprise preventing movement of the plunger rod towards the retracted plunger rod position for a dwell time, e.g. in order to prevent back flow of medicament. Alternatively or additionally, adjusting 3010 the movement may comprise maintaining the position of the plunger rod for a dwell time, e.g. in order to prevent back flow of medicament. Alternatively or additionally, adjusting 3010 the movement may comprise moving the plunger rod to the retracted plunger rod position. Alternatively or additionally, adjusting 3010 the movement may comprise decreasing the plunger rod speed.

Adjusting 3010 the movement of the plunger rod may be based on the resistance signal. For example, the movement of the plunger rod may be adjusted such that the resistance is held below a high resistance threshold. Adjusting 3010 the movement of the plunger rod may comprise adjusting the movement of the plunger rod if the resistance signal is indicative of resistance against movement of the plunger rod above a high resistance threshold. The high resistance threshold may be based on the plunger rod position, e.g. the high resistance threshold may be a first high resistance threshold when the plunger rod position is within a one range, and a second high resistance threshold when the plunger rod position is within a second range.

Steps of the exemplary method 3000, e.g. the steps of moving 3002 a plunger rod; determining 3004 plunger rod position; receiving a resistance signal 3006; and adjusting 3010 the movement of the plunger rod, may be controlled by a processing unit, such as the processing unit of the auto injector.

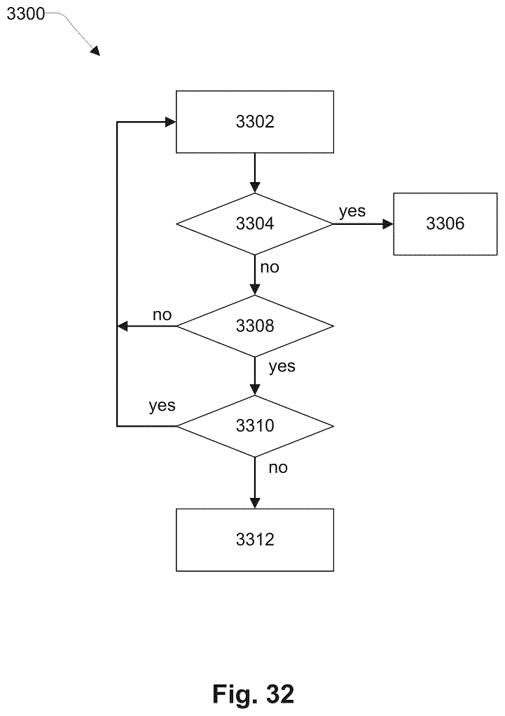

FIG. 32 shows a flow chart of an exemplary method 3300 for moving the plunger rod of an auto injector.

Initially, the plunger rod is moved 3302, e.g. with a first plunger rod speed, e.g. in a first plunger rod direction.

The resistance against the movement of the plunger rod is monitored, such as continuously monitored. By a first resistance criterion 3304, it is determined whether resistance against movement of the plunger rod exceeds a second high resistance threshold. If the resistance against movement of the plunger rod does not exceed the second high resistance threshold (first resistance criterion 3304 is answered no), by a second resistance criterion 3308, it is determined whether resistance against movement of the plunger rod exceeds a first high resistance threshold. If the resistance against movement of the plunger rod does not exceed the first high resistance threshold (second resistance criterion 3304 is answered no), the movement of the plunger rod is continued 3302. The first plunger threshold may be lower than the second high resistance threshold.

The position of the plunger rod is monitored, such as continuously monitored. If the resistance against movement of the plunger rod does exceed the first high resistance threshold (second resistance criterion 3308 is answered yes), by a first position criterion 3310, it is determined whether the plunger rod has reached and/or passed a predetermined plunger rod position, such as a first plunger rod position, a second plunger rod position, a third plunger rod position, a fourth plunger rod position and/or a fifth plunger rod position (see e.g. FIGS. 28 and 30 for exemplary positions). If the plunger rod position has reached and/or passed the predetermined plunger rod position (first position criterion 3310 is answered yes), the movement of the plunger rod is continued 3302. Thus, the first high resistance threshold may be exceeded if the plunger rod has reached and/or passed the predetermined plunger rod position.

If the plunger rod position has not reached and/or passed the predetermined plunger rod position (first position criterion 3310 is answered no), the movement of the plunger rod is stopped 3312, and an error may be communicated to the user, e.g. via a user interface. Thus, an error may be assumed if the first high resistance threshold is exceeded before the plunger rod has reached and/or passed the predetermined plunger rod position.

If the resistance against movement of the plunger rod does exceed the second high resistance threshold (first resistance criterion 3304 is answered yes), the movement of the plunger rod is stopped 3306 and end of injection may be assumed. In stopping 3306 the movement of the plunger rod, the plunger rod may be locked in its position for a dwell time, e.g. to prevent a sudden drop in pressure in the cartridge, e.g. to prevent back flow of medicament

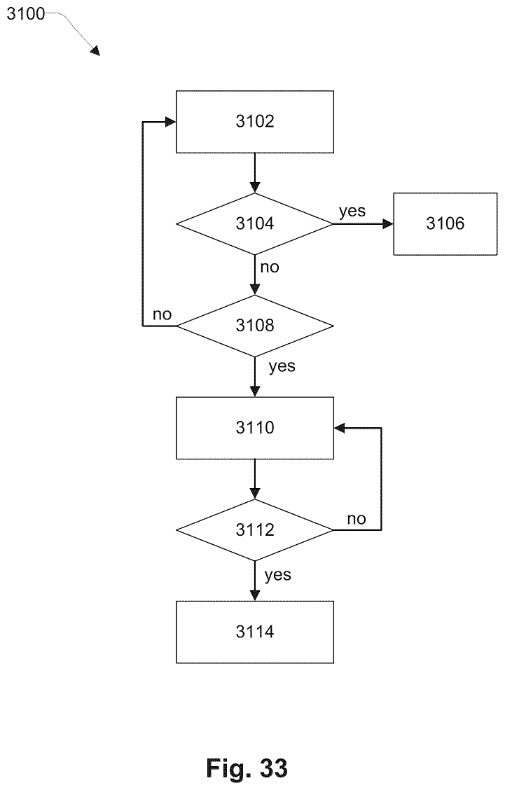

FIG. 33 shows a flow chart of an exemplary method 3100 for moving the plunger rod of an auto injector.

Initially, the plunger rod is moved 3102 with a first plunger rod speed, e.g. in a first plunger rod direction.

The resistance against the movement of the plunger rod is monitored, such as continuously monitored. By a first resistance criterion 3104, it is determined whether resistance against movement of the plunger rod exceeds a first high resistance threshold. If the resistance against movement of the plunger rod does exceed the first high resistance threshold (first resistance criterion 3104 is answered yes), the movement of the plunger rod is stopped 3106 and an error may be communicated to the user, e.g. via a user interface.

The position of the plunger rod is monitored, such as continuously monitored. If the resistance against movement of the plunger rod does not exceed the first high resistance threshold (first resistance criterion 3104 is answered no), by a first position criterion 3108, it is determined whether the plunger rod has reached and/or passed a predetermined plunger rod position, such as a first plunger rod position, a second plunger rod position, a third plunger rod position, a fourth plunger rod position and/or a fifth plunger rod position (see e.g. FIGS. 28 and 30 for exemplary positions). If the plunger rod position has not reached and/or passed the predetermined plunger rod position (first position criterion 3108 is answered no), the movement of the plunger rod is continued 3102 with the first plunger rod speed.

If the plunger rod position has reached and/or passed the predetermined plunger rod position (first position criterion 3108 is answered yes), the plunger rod is moved 3110 with a second plunger rod speed, e.g. in the first plunger rod direction. The second plunger rod speed may be lower than the first plunger rod speed. By lowering the plunger rod speed, the amount of medicament needing to be forced though the needle per time, is reduced, thereby reducing the amount of force needed to advance the stopper.

By a second resistance criterion 3112, it is determined whether resistance against movement of the plunger rod exceeds a second high resistance threshold. If the resistance against movement of the plunger rod does not exceed the second high resistance threshold (second resistance criterion 3112 is answered no), the movement of the plunger rod is continued 3110 with the second plunger rod speed.

If the resistance against movement of the plunger rod does exceed the second high resistance threshold (second resistance criterion 3112 is answered yes), the movement of the plunger rod is stopped 3114 and end of injection may be assumed. In stopping 3114 the movement of the plunger rod, the plunger rod may be locked in its position for a dwell time, e.g. to prevent a sudden drop in pressure in the cartridge, e.g. to prevent back flow of medicament.

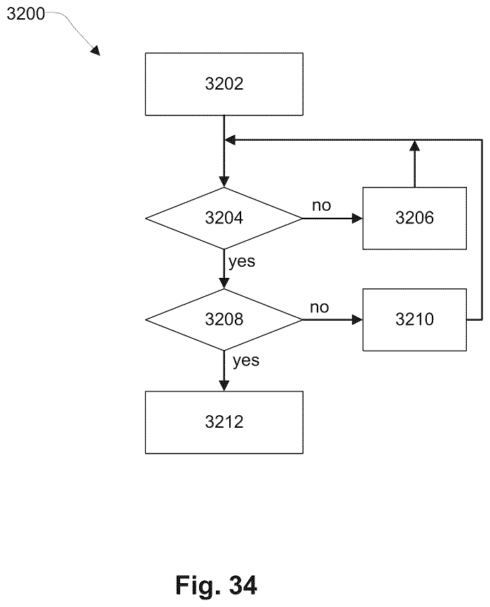

FIG. 34 shows a flow chart of an exemplary method 3200 for moving the plunger rod of an auto injector.

Initially, the plunger rod is moved 3202, e.g. with a first plunger rod speed, e.g. in a first plunger rod direction.

The resistance against the movement of the plunger rod is monitored, such as continuously monitored. By a resistance criterion 3204, it is determined whether resistance against movement of the plunger rod exceeds a high resistance threshold, such as a first high resistance threshold and/or a second high resistance threshold.

If the resistance against movement of the plunger rod does not exceed the high resistance threshold (resistance criterion 3204 is answered no), the speed of the movement of the plunger rod is increased 3206.

If the resistance against movement of the plunger rod does exceed the high resistance threshold (resistance criterion 3204 is answered yes), it is determined by a speed criteria 3208 whether the plunger rod speed is zero, i.e. the plunger rod is not moving. If the plunger rod speed is not zero (speed criteria 3208 is answered no) the plunger rod speed is reduced 3210. If the plunger rod speed is zero (speed criteria 3208 is answered yes) the process is stopped 3212. In stopping 3212, the plunger rod may be locked in its position for a dwell time, e.g. to prevent a sudden drop in pressure in the cartridge, e.g. to prevent back flow of medicament.

The high resistance threshold of resistance criterion 3204 may be determined based on the position of the plunger rod. The plunger rod position may also be used to determine whether in stopping 3212 the process, the medicament has been expelled sufficiently and/or an error caused the process to stop too early. A corresponding message may be provided to the user, e.g. via the user interface.

By the method 3200, the speed is adjusted to be as high as possible without exceeding the resistance thresholds.

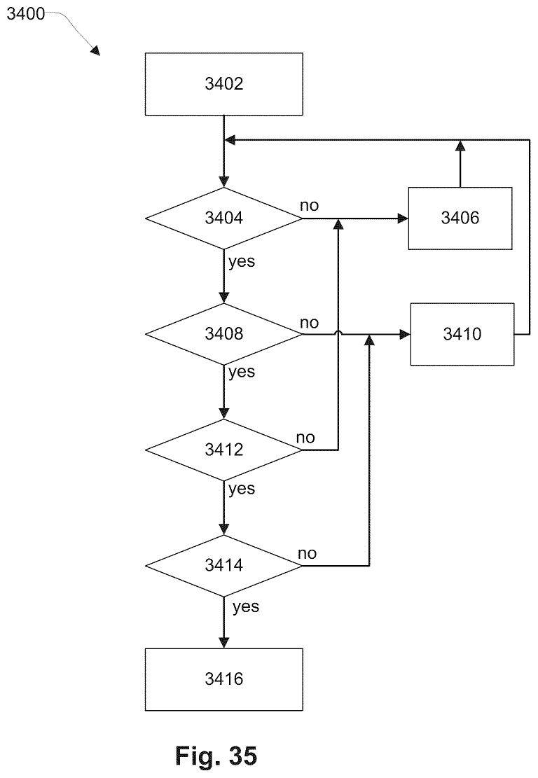

FIG. 35 shows a flow chart of an exemplary method 3400 for moving the plunger rod of an auto injector.

Initially, the plunger rod is moved 3402, e.g. with a first plunger rod speed, e.g. in a first plunger rod direction.

The resistance against the movement of the plunger rod is monitored, such as continuously monitored. By a first resistance criterion 3404, it is determined whether resistance against movement of the plunger rod exceeds a first high resistance threshold.

If the resistance against movement of the plunger rod does not exceed the first high resistance threshold (first resistance criterion 3404 is answered no), the speed of the movement of the plunger rod is increased 3406.

The position of the plunger rod is monitored, such as continuously monitored. If the resistance against movement of the plunger rod does exceed the first high resistance threshold (first resistance criterion 3404 is answered yes), by a first position criterion 3408, it is determined whether the plunger rod has reached and/or passed a predetermined plunger rod position, such as a first plunger rod position, a second plunger rod position, a third plunger rod position, a fourth plunger rod position and/or a fifth plunger rod position (see e.g. FIGS. 28 and 30 for exemplary positions).

If the plunger rod position has not reached and/or passed the predetermined plunger rod position (first position criterion 3408 is answered no), the speed of the movement of the plunger rod is decreased 3410.

If the plunger rod position has reached and/or passed the predetermined plunger rod position (first position criterion 3408 is answered yes), the movement of the plunger rod may be continued. Thus, the first high resistance threshold may be exceeded if the plunger rod has reached and/or passed the predetermined plunger rod position. In this case, by a second resistance criterion 3412, it is determined whether resistance against movement of the plunger rod exceeds a second high resistance threshold.

If the resistance against movement of the plunger rod does not exceed the second high resistance threshold (second resistance criterion 3412 is answered no), the speed of the movement of the plunger rod is increased 3406.

If the resistance against movement of the plunger rod does exceed the second high resistance threshold (second resistance criterion 3412 is answered yes), it is determined by a speed criteria 3414 whether the plunger rod speed is zero, i.e. the plunger rod is not moving.

If the plunger rod speed is not zero (speed criteria 3414 is answered no) the plunger rod speed is reduced 3410. If the plunger rod speed is zero (speed criteria 3414 is answered yes) the process is stopped 3416. In stopping 3416, the plunger rod may be locked in its position for a dwell time, e.g. to prevent a sudden drop in pressure in the cartridge, e.g. to prevent back flow of medicament. In stopping 3416 end of injection may be assumed.

By the method 3400, the speed is adjusted to be as high as possible without exceeding the resistance thresholds.

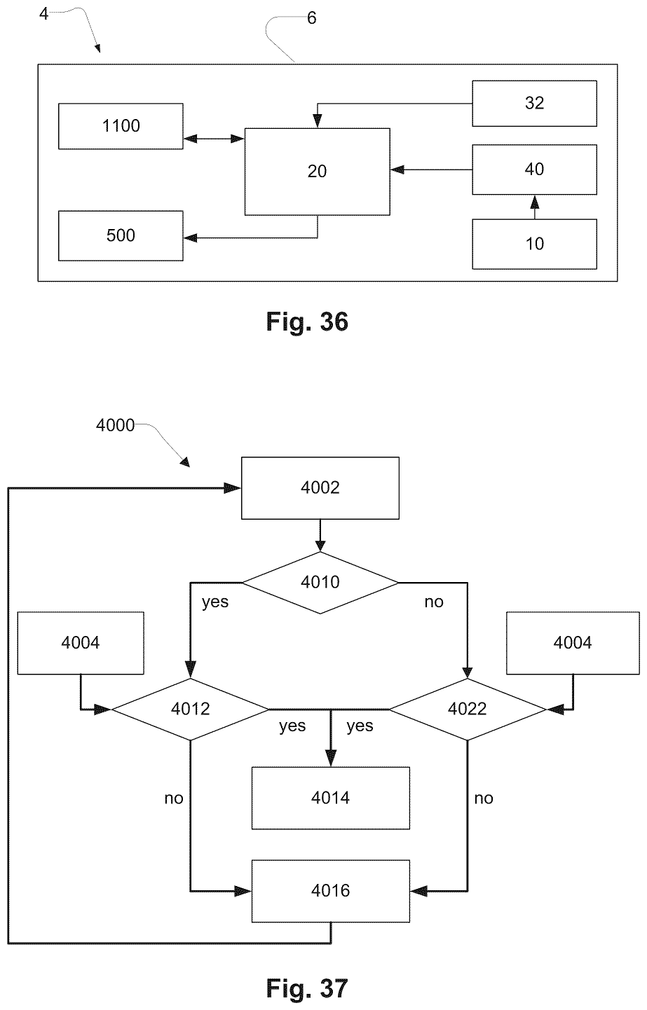

FIG. 36 shows a block diagram of an exemplary auto injector 4. The auto injector 4 comprises a re-chargeable battery 10, a battery calculation module 40 configured to calculate a residual electrical battery voltage level of the re-chargeable battery 10, thereby proving a measure of the remaining residual electrical battery voltage level, a drive module 500, a temperature sensor 32 configured to measure a temperature of the auto injector 4, a processing unit 20, and a user interface 1100. The user interface 1100, the temperature sensor 32, the drive module 500, and the battery calculation module 40 are coupled to the processing unit 20. The processing unit 20 is also coupled to the drive module 500.

By temperature of the auto injector is meant a temperature measured by the auto injector temperature sensor anywhere inside the auto injector. In one or more examples, the temperature of the auto injector is one or more of:
an ambient temperature; and/or
a temperature near the medicament in the cartridge; and/or
a temperature indicative of a temperature of the medicament in the cartridge; and/or
a temperature of the auto injector near the battery; and/or
a temperature indicative of the temperature of the battery; and/or
any combination of the above.

The processing unit 20 receives a value of the measured temperature from the temperature sensor 32. The processing unit 20 also receives a value of the calculated residual electrical battery voltage level of the re-chargeable battery 10 from the battery calculation module 40. Based on at least these two values, the processing unit 20 obtains a predefined threshold value indicative of the minimum electrical battery voltage level needed for performing an auto injector process at the temperature measured by the temperature sensor.

The auto injector process may be one or more of:
a first plunger rod movement process, where the plunger rod 400 is moved from the retracted plunger rod position to a locking plunger rod position, where a cartridge 700 is locked inside the auto injector 4;
a second plunger rod movement process, where the plunger rod 400 is moved from the locking plunger rod position to a first stopper plunger rod position bringing the plunger rod 400 in contact with the first stopper 708;
a third plunger rod movement process, where the plunger rod 400 moves the first stopper 708 to cause a second stopper 710 inside the cartridge to move to a bypass section 712 for establishing a fluid connection between a first cartridge sub-compartment 704 and a second cartridge sub-compartment 706 inside the cartridge compartment 702;
a fourth plunger rod movement process, where the plunger rod 400 moves the first stopper 708 to contact the second stopper 710 for mixing of medicament components the first cartridge sub-compartment 704 and the second cartridge sub-compartment 706;
a fifth plunger rod movement process, where the plunger rod 400 is moved to the extended plunger rod position, where at the extended plunger rod position, medicament has been expelled from the cartridge, such as fully expelled from the cartridge;
a re-setting of the auto injector 4 to an original position where the cartridge 700 can be removed from the auto injector;
a full medicament delivery process comprising a combination of the above processes.

The auto injector process may be a medicament reconstitution process and/or a medicament expelling process, or similar. For example, the auto injector process may a full injection cycle including the medicament reconstitution process, the medicament expelling process, and a re-setting of the auto injector to an original position where the cartridge can be removed from the auto injector thereby allowing for insertion of a new cartridge in the auto injector.

The predefined threshold value may be defined to be the minimum voltage level needed to perform a full injector cycle. Alternatively, the predefined threshold value may be obtained indirectly by calculating the estimated count of remaining injection cycles without a recharging of the battery.

The processing unit 20 further compares the calculated residual electrical battery voltage level with the obtained predefined threshold value.

If the comparison results in the processing unit 20 finding the residual electrical battery voltage level to be larger than the predefined threshold value needed to perform the auto injector process at the measured temperature, the auto injector will initiate the auto injector process.

Alternately, if the comparison results in the processing unit 20 finding the residual electrical battery voltage level to be smaller than the predefined threshold value needed to perform the auto injector process at the measured temperature, the auto injector will instruct the user to recharge the battery 10. The processing unit 20 may send a signal to the user interface 1100 with instructions/information for the user.

FIG. 37 shows a flow chart of a method 4000 for determining whether battery voltage level is sufficiently high for the processing unit 20 to allow the auto injector process to proceed. The method alternatively detects whether the battery need to be recharged first. The method 4000 may be run when the auto injector is turned on.

The method 4000 comprises measuring 4002 the temperature of the auto injector 4, e.g. the temperature close to the re-chargeable battery, by means of the temperature sensor 32. A measure 4004 of the battery voltage level is also measured in the same sequence by means of the battery calculation module 40.

The method 4000 further comprises determining 4010 if the temperature is above a predetermined threshold temperature, such as 15 degrees Celsius or such as 12 degrees Celsius. If the temperature is above the predetermined threshold temperature (yes option in 4010), the processing unit determines 4012 if there is sufficient electrical battery voltage level to perform an auto injector process, e.g. a full injection cycle. The determination is performed by comparing the measured battery voltage level indicative of the residual battery voltage level, with the predefined threshold value needed to perform the auto injector process at the measured temperature.

If there is sufficient electrical battery voltage level to perform the auto injector process (yes option in 4012), the processing unit 20 communicates to the user 4014 that the auto injector is ready for use, i.e. the auto injector process may proceed.

If there is not sufficient electrical battery voltage level to perform the auto injector process (no option in 4012), the processing unit communicates to the user 4016 that the battery needs to be recharged before the auto injector is ready for use, i.e. before the user may perform the auto injector process. Before the auto injector process can be initiated, the method 4000 is repeated.

If the temperature measured in 4002 is determined to be below the predetermined threshold temperature (no option in 4010), the processing unit determines 4022 if the electrical battery voltage level is sufficient to perform not one, but two auto injector processes, e.g. two full injection cycles.

If the electrical battery voltage level is sufficient to perform two auto injector processes (yes option in 4022), the processing unit communicates 4014 to the user that the auto injector is ready for use, i.e. the auto injector process may proceed.

If the electrical battery voltage level is not sufficient to perform two auto injector processes (no option in 4022), the processing unit communicates 4016 to the user to re-charge the battery before the auto injector is ready for use. Before the auto injector process can be initiated, the method 4000 is repeated. The temperature is normally not measured again till the devices makes a new self-test validation, i.e. runs the method 4000 again.

For temperatures below the predetermined threshold temperature, e.g. 15 or 12 degrees Celsius, the battery voltage threshold may be 3850 mV voltage, meaning that measuring to confirm this voltage will ensure operational battery capacity to a minimum of two injection cycles. A voltage below this threshold would be interpreted as capacity less than two injection cycles.

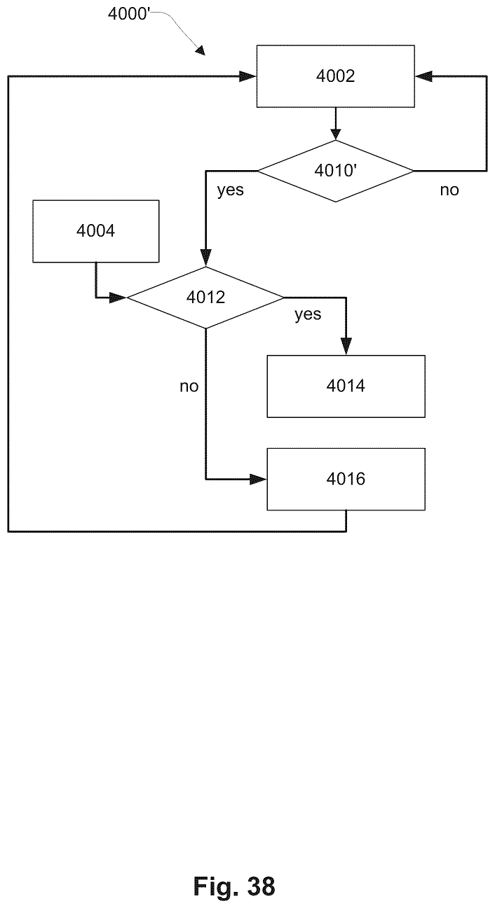

In an alternative method 4000′ as illustrated in FIG. 38, the auto injector may be configured to require the temperature to be above a first temperature threshold value before the auto injector will be allow to start the auto injector process. If the temperature measured in 4002 is found to be below the first threshold temperature, such as 15 degrees Celsius or such as 12 degrees Celsius in 4010′ (no option in 4010′), the auto injector will prevent an initiation of the auto injector process. Before the auto injector process can be initiated, the method 4000 is repeated until the temperature is measured to be above the first temperature threshold value. If the temperature measured in 4002 is found to be above the first threshold temperature, in 4010′ (yes option in 4010′), the process proceeds as described in FIG. 37.

The temperature sensor may sit within a Nordic Semiconductor nRF8001 chip as part of Bluetooth chip. The voltages responsible for determining remaining battery charge level may be measured using the ADC input on the micro-processor, e.g. a Atmel ATXmega256A3U-MH processor.

The medicament may be a human growth hormone. However, this is only an exemplary use of the auto injector. The medicament may be a depot version or prodrug, such as a long-acting version, of human growth hormone. The medicament may be lonapegsomatropin. The second medicament component may be a dry composition of human growth hormone.

The viscosity of the human growth hormone lonapegsomatropin reconstituted medicament product solution is increased compared to e.g. water. The viscosity of the reconstituted medicament product solution impacts the performance of the auto injector. Hydraulic pressure loss e.g. impacts the injection time at constant drug product solution pressure and the hydraulic pressure loss depends on the viscosity. Hence viscosity influences the injection time.

The viscosity is also likely to influence mixing dynamics during the reconstitution process, and hence impacts the time needed for the reconstitution performed with the auto injector. Three viscosities at different strength of the human growth hormone (hGH) lonapegsomatropin are summarized in Table 1.

TABLE 1
Lonapegsomatropin medicament viscosities given as mg hGH.

| | Medicament Solution | Viscosity |
|---|---|---|
| Strength | Concentration | (at 25° C.) |
| mg hGH | mg hGH/mL | mPa · s |
| 0.7 | 2.14 | 1.3 |
| 3.0-5.2 | 11.0 | 3.13 |
| 6.3-16.0 | 22.0 | 7.53 |

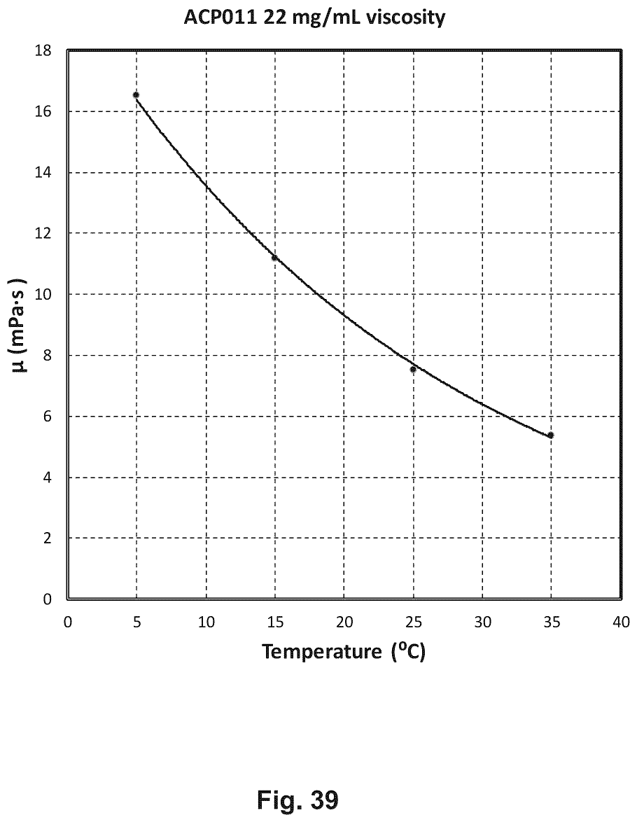

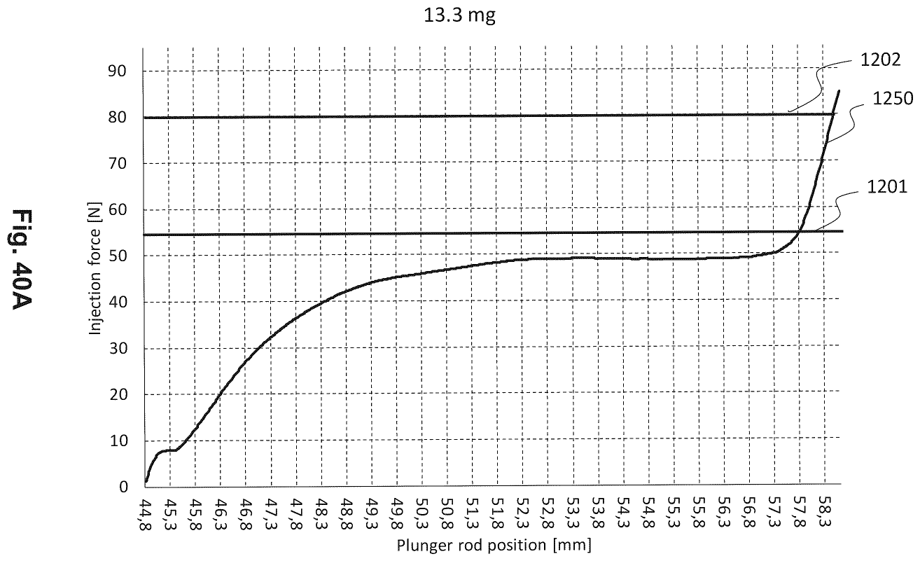

The viscosity of lonapegsomatropin is shown in table 1 at 25 degrees Celsius. However, as seen in FIG. 39 showing the temperature dependence of the viscosity of lonapegsomatropin in a medicament solution concentration of 22.0 mg/mL hGH, the viscosity increases significantly when the temperature is lowered. Thus, the viscosity of the reconstituted lonapegsomatropin medicament solution is strongly temperature dependent. Hence the requirement to the battery voltage level needed for performing an auto injector process such as e.g. a full injection cycle, depends strongly on the temperature of the auto injector and the medicament.

Table 2 shows specifics on the auto injector for delivery of lonapegsomatropin at different lonapegsomatropin medicament solution concentrations.

TABLE 2
Auto injector and lonapegsomatropin medicament specificities.

| | lonapegsomatropin Solution Concentration | | | | | |
|---|---|---|---|---|---|---|
| | 13.3 mg | | 13.3 mg | | 5.2 mg | |
| | hGH/mL | | hGH/mL | | hGH/mL | |
| Temperature | 25° | C. | 15° | C. | 25° | C. |
| Viscosity | 7.53 | mPa · s | 11.2 | mPa · s | 3.13 | mPa · s |
| Volume | 0.605 | ml | 0.605 | ml | 0.473 | ml |
| Cartridge type | Dual chamber cartridge V-LK S ®/Vetter | | | | | |
| | Pharma Fertigung GmbH & Co. KG | | | | | |
| Needle | Omnican ®fine 0.25 mm × 4 mm (31G × 5/32)/ | | | | | |
| | B Braun, Melsungen Germany | | | | | |
| First high resistance | 55N | | 55N | | 55N | |
| threshold | | | | | | |
| Second high | 80N | | 80N | | 80N | |
| resistance threshold | | | | | | |
| Injection Time | 6.9 | s | 10.8 | s | 5.4 | s |

The auto injector attempts a constant stopper velocity of 90 mm/min during the whole of an injection cycle. When doing so, the auto injector monitors the plunger rod force (motor current). If it exceeds 55 N (and 80 N in last stage), the auto injector reduces the plunger rod velocity until the plunger rod force is within an acceptable level. Whether the condition is met depends on the actual stopper friction which may vary from approximately 6 N to 22 N. The plunger rod force further depends on the actual inner needle diameter of the needle, since the resistance scales with the inner diameter to fourth power.

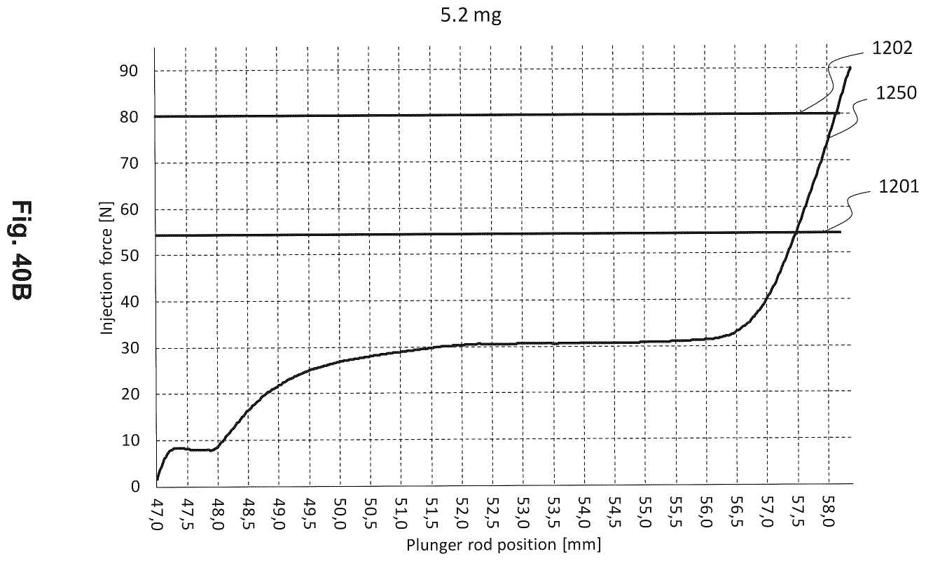

FIG. 40A and FIG. 40B show a measure of the injection force 1250 needed as a function of the plunger rod position when the cartridge contains, respectively, a 13.3 mg hGH/mL and a 5.2 mg hGH/mL lonapegsomatropin medicament solution. The first high resistance threshold 1201 and the second high resistance threshold 1202 are also illustrated in both figures. As seen by comparing FIG. 40A and FIG. 40B, the injection force depends strongly on the concentration of the lonapegsomatropin medicament solution, which as seen in Table. 1 is directly correlated to the viscosity of the lonapegsomatropin medicament solution. Thus, the higher the concentration of the lonapegsomatropin medicament solution—and thereby also the viscosity of the lonapegsomatropin medicament solution—is, the higher the needed injection force—and thereby also the battery power requirement—is.

Although particular features have been shown and described, it will be understood that they are not intended to limit the claimed invention, and it will be made obvious to those skilled in the art that various changes and modifications may be made without departing from the spirit and scope of the claimed invention. The specification and drawings are accordingly to be regarded in an illustrative rather than restrictive sense. The claimed invention is intended to cover all alternatives, modifications and equivalents.

### REFERENCES

2 system
4 auto injector
6 housing
10 battery
12 first electrical connector
14 connector opening
18 second electrical connector
20 processing unit
22 orientation sensor
24 code sensor
26 plunger rod position sensor
28 cartridge sensor
30 needle sensor
32 temperature sensor
34 resistance sensor
40 battery calculation module
100, 100′ blocking member
102 first blocking coupling member
104 first blocking member stop
106 the second blocking member stop
200 ejector
202 ejector member
204 ejector abutment face
206 end portion
207 angle retaining guide
208 second blocking coupling member
212 ejector lock
214 bottom portion
216 ejector lock guide pin
218 ejector resilient member
222 ejector rod bore
224 ejector collar
226 ejector cog
228 ejector cut-out
230 ejector lock cut-out
232 ejector lock cog
234 end portion
236 ejector spring
240 ejector lock bore
250 stop position
251 extreme position
300 cartridge receiver
301 cartridge receiver opening
302 cartridge receiver compartment
304 receiving direction
310 first section
312 first guide members
314 first guide member angle
316 passage
318 passage angle
322 second guide members
324 first face
326 second face
328 retention portion
330 second section
332 retention angle
334 slope portion
336 slope angle
338 eject face
340 first riser portions
342 second riser portion
344 section
346 section
348 retention face
350 bore
352 opening
354 base
356 bore
400 plunger rod
402 inner plunger rod
404 outer plunger rod
406 plunger rod track
408 plunger rod track
410 plunger rod front end
424 plunger rod distal rim
428 first track portion
430 third track portion
432 second track portion
500 drive module
502 motor
504 transmission
600 cartridge assembly
700 cartridge
701 dual chamber cartridge
702 cartridge component
704 first cartridge subcomponent
706 second cartridge subcomponent
708 first stopper
710 second stopper
712 bypass section
714 cartridge outlet
716 cartridge back face
718 first end
720 second end
722 first stopper direction
790 medicament
792 first medicament component
794 second medicament component
800 cartridge holder
806 cartridge assembly outlet opening
808 cartridge retention member
812 needle assembly coupling portion
814 cartridge holder slot
900 needle assembly
902 needle
904 needle hub
906 cartridge holder coupling portion
908 needle cover
910 needle cover abutment face
1000 cartridge code feature
1100 user interface
1102 contact member
1104 contact member sensor
1106 first LED
1108 first input member
1110 first output member
1112 contact member protruding part
1114 needle cover engagement member
1130 first contact member sensor
1132 second contact member sensor
1200 resistance graph
1200X position axis
1200Y resistance axis
1201 first high resistance threshold
1202 second high resistance threshold
1204 third high resistance threshold
1206 first slope
1208 second slope
1210 third slope
1220 first plunger rod position
1222 second plunger rod positon
1223 third plunger rod position
1224 fourth plunger rod position
1226 fifth plunger rod position
1228 retracted plunger rod position
1229 extended plunger rod position
1240 first plunger rod speed
1242 second plunger rod speed
1250 injection force as a function of the plunger rod position
1300 speed graph
1300X position axis
1300Y speed axis
3000 method
3001 receiving
3002 moving
3004 determining
3006 receiving
3010 adjusting
3100 method
3102 move plunger rod at first speed
3104 first resistance criteria
3106 stop movement of plunger rod
3108 first position criteria
3110 move plunger rod at second speed
3112 second resistance criteria
3114 stop movement of plunger rod
3200 method
3202 move plunger rod at first speed
3204 resistance criteria
3206 increase speed
3208 speed criteria
3210 decrease speed
3212 stop movement of plunger rod
3300 method
3302 move plunger rod at first speed
3304 first resistance criteria
3306 stop movement of plunger rod
3308 second resistance criteria
3310 first position criteria
3312 stop movement of plunger rod
3400 method
3402 move plunger rod at first speed
3404 first resistance criteria
3406 increase speed
3408 first position criteria
3410 decrease speed
3412 second resistance criteria
3414 speed criteria
3416 stop movement of plunger rod
4000, 4000′ method
4002 measure the temperature of the auto injector
4004 measure the battery voltage level
4010, 4010′ temperature above a predetermined threshold temperature
4012 sufficient electrical voltage to perform an auto injector process
4014 auto injector is ready for use
4016 battery recharging needed
4022 sufficient electrical voltage to perform two auto injector processes

1. An auto injector for administering a medicament, the auto injector comprising:
a cartridge receiver configured to receive a cartridge, the cartridge comprising a cartridge outlet, a cartridge compartment containing the medicament, and a first stopper;
a plunger rod configured to move the first stopper inside the cartridge compartment for expelling the medicament through the cartridge outlet;
a drive module configured to move the plunger rod from a retracted plunger rod position to an extended plunger rod position;
a temperature sensor configured to measure a temperature of the auto injector;
a re-chargeable battery configured to power at least the drive module when moving the plunger rod;
a battery calculation module configured to calculate a residual electrical battery voltage level of the re-chargeable battery;
a processing unit coupled to the temperature sensor, the battery calculation module, and the drive module;
wherein the processing unit is configured to:
receive the measured temperature from the temperature sensor;
receive the calculated residual electrical battery voltage level of the re-chargeable battery from the battery calculation module;
obtain a predefined threshold value indicative of the minimum electrical battery voltage level needed for performing an auto injector process at the temperature measured by the temperature sensor;
compare the calculated residual electrical battery voltage level with the obtained predefined threshold value; and
initiate the auto injector process only if the calculated residual electrical battery voltage level is larger than the predefined threshold value.

2-35. (canceled)

36. The auto injector according to claim 1, wherein the processing unit is further configured to instruct the user to recharge the battery if the calculated residual electrical battery voltage level is smaller than the predefined threshold value needed to perform the auto injector process at the measured temperature.

37. The auto injector according to claim 1, wherein the temperature sensor, the battery calculation module, and the processing unit are configured to perform the steps outlined in claim 1 and to instruct the user to recharge the battery if the calculated residual electrical battery voltage level is smaller than the predefined threshold value needed to perform the auto injector process at the measured temperature without a cartridge being received in the auto injector.

38. The auto injector according to claim 1, wherein the auto injector process is one or more of:
a first plunger rod movement process, where the plunger rod is moved from the retracted plunger rod position to a locking plunger rod position, where a cartridge is locked inside the auto injector;
a second plunger rod movement process, where the plunger rod is moved from the locking plunger rod position to a first stopper plunger rod position bringing the plunger rod in contact with the first stopper;
a third plunger rod movement process, where the plunger rod moves the first stopper to cause a second stopper inside the cartridge to move to a bypass section for establishing a fluid connection between a first cartridge sub-compartment and a second cartridge sub-compartment inside the cartridge compartment;
a fourth plunger rod movement process, where the plunger rod moves the first stopper to contact the second stopper for mixing of medicament components the first cartridge sub-compartment and the second cartridge sub-compartment;
a fifth plunger rod movement process, where the plunger rod is moved to the extended plunger rod position, where at the extended plunger rod position, medicament has been expelled from the cartridge;
a re-setting of the auto injector to an original position where the cartridge can be removed from the auto injector; and
a full medicament delivery process comprising a combination of the above processes.

39. The auto injector according to claim 1, wherein the temperature of the auto injector is one or more of:
an ambient temperature; and/or
a temperature near the medicament in the cartridge; and/or
a temperature indicative of a temperature of the medicament in the cartridge; and/or
a temperature of the auto injector near the battery; and/or
a temperature indicative of the temperature of the battery; and/or
any combination of the above.

40. The auto injector according to claim 1, wherein the predefined threshold value indicative of the electrical voltage needed for performing the auto injector process is set to a first fixed threshold value for temperatures above a predetermined threshold temperature, and to a second fixed threshold value for temperatures at or below the predetermined threshold temperature.

41. The auto injector according to claim 40, wherein the second threshold value is two times higher than the first threshold value.

42. The auto injector according to claim 40, wherein the second threshold value is between 3000 MV and 4500 MV.

43. The auto injector according to claim 40, wherein the predetermined threshold temperature is at or below 15 degrees Celsius.

44. The auto injector according to claim 1, wherein the predefined threshold value increases when the measured temperature decreases.

45. The auto injector according to claim 40, wherein the processing unit is further configured to prevent initiating of the auto injector process if the measured temperature is at or below the predetermined threshold temperature.

46. The auto injector according to claim 1, wherein the initiation of the auto injector process only occurs if the calculated residual electrical battery voltage level is larger than the predefined threshold value by at least a predetermined tolerance value.

47. The auto injector according to claim 46, wherein the predetermined tolerance value is at least 5% larger than the minimum electrical battery voltage level needed to performing the auto injector process at the measured temperature.

48. The auto injector according to claim 46, wherein the predetermined tolerance value increases with decreasing temperature.

49. The auto injector according to claim 1, wherein the cartridge further comprises a cartridge code feature, the cartridge code feature comprising information indicative of at least the medicament viscosity at at least one predefined temperature, and wherein the auto injector further comprises a cartridge code sensor coupled to the processing unit and configured to receive the information indicative of at least the medicament viscosity at at least one predefined temperature from the cartridge code feature when a cartridge is received in the auto injector.

50. The auto injector according to claim 49, wherein the predefined threshold value indicative of the minimum electrical battery voltage level needed for performing the auto injector process at the measured temperature is also depending on the medicament viscosity, and wherein the processing unit is further configured to:
receive information indicative of at least the medicament viscosity at at least one predefined temperature from the cartridge code feature;
obtain the predefined threshold value indicative of the minimum value of the electrical voltage needed for performing the auto injector process based both on the temperature as measured by the temperature sensor and on the information indicative of at least the medicament viscosity at at least one predefined temperature from obtained from the cartridge code feature.

51. The auto injector according to claim 49, wherein the medicament viscosity is temperature dependent, and wherein the viscosity of medicament at any specific temperature is stored in the cartridge code feature as a curve.

52. The auto injector according to claim 1, wherein the auto injector further comprises a user interface coupled to the processing unit, wherein the processing unit is configured to instruct a user via the user interface to re-charge the battery if:
A) the difference between the calculated residual electrical battery voltage level and the predefined threshold value indicative of the minimum electrical battery voltage level needed for performing the auto injector process at the measured temperature is smaller than the predetermined tolerance value, or
B) the calculated residual electrical battery voltage level is smaller than the predefined threshold value at the measured temperature.

53. The auto injector according to claim 52, wherein the user interface comprises a plurality of LEDs including a first LED, and wherein the user interface instructs the user to re-charge the battery by flashing the first LED.

54. The auto injector according to claim 53, wherein the first LED flashes until:
A) the difference between the calculated residual electrical battery voltage level and the predefined threshold value indicative of the minimum electrical battery voltage level needed for performing the auto injector process at the measured temperature is larger than the predetermined tolerance value, or
B) the calculated residual electrical battery voltage level is larger than the predefined threshold value at the measured temperature.

55. The auto injector according to claim 1, wherein the battery calculation module is configured for calculating at least one of:
a time since the re-chargeable battery was last charged;
an age of the re-chargeable battery; and
an electrical voltage used since the re-chargeable battery was last charged;
wherein the processing unit is configured to obtain an updated predefined threshold value indicative of the minimum electrical battery voltage level needed for performing the auto injector process at the measured temperature based on at least one of:
the time since the re-chargeable battery was last charged;
the age of the re-chargeable battery; and
the electrical voltage used since the re-chargeable battery was last charged.

56. The auto injector according to claim 1, wherein the auto injector further comprises a temperature control unit coupled to the processing unit, wherein the temperature control unit is configured to heat the auto injector, and wherein the processing unit is further configured to:
obtain the measured temperature measured by the temperature sensor;
initiate a heating of the auto injector if the measured temperature is below a predetermined heating-initiation temperature;
obtain a second measure of measured temperature measured by the temperature sensor; and
stop the heating of the auto injector when the second temperature is above a predetermined heating-stop temperature and/or after the auto injector has been heated for a predetermined time.

57. The auto injector according to claim 1, wherein the cartridge outlet is an injection needle having an inner needle diameter, and wherein the predefined threshold value indicative of the electrical voltage needed for performing the auto injector process at the measured temperature depends on the inner needle diameter.

58. The auto injector according to claim 57, wherein the inner needle diameter is between 145 μm and 160 μm.

59. The auto injector according to claim 57, wherein the needle is a 31 gauge needle.

60. The auto injector according to claim 1, wherein movement of the plunger rod from the retracted plunger rod position to a locking plunger rod position positioned between the retracted plunger rod position and the extended plunger rod position locks the cartridge inside the auto injector, wherein the processing unit is further configured for preventing movement of the plunger rod to the locking plunger rod position if:
the calculated residual electrical voltage of the re-chargeable battery is smaller than the value of the electrical voltage needed for performing the auto injector process at the measured temperature, or
the difference between the calculated residual electrical voltage of the re-chargeable battery and the value of the electrical voltage needed for performing the auto injector process at the measured temperature is smaller than the predetermined tolerance value.

61. The auto injector according to claim 1, wherein the auto injector further comprises:
a housing accommodating the cartridge receiver, the plunger rod, the drive module, the temperature sensor, the re-chargeable battery, the battery calculation module (40) and the processing unit;
a connector opening in the housing allowing the auto injector to be connectable to an electrical voltage supply for re-charging the re-chargeable battery;
an elongated ejector comprising an ejector member movable along a longitudinal axis between a first ejector position and a second ejector position and being configured to follow movement of the cartridge along the longitudinal axis when the cartridge is received in the cartridge receiver;
a blocking member coupled to the ejector member (202), the blocking member being configured to move between a blocking position wherein the connector opening is blocked and a non-blocking position wherein the connector opening is not blocked, wherein the blocking member is in the blocking position when the ejector member is in the second ejector position, and wherein the blocking member is in the non-blocking position when the ejector member is in the first ejector position.

62. The auto injector according to claim 61, wherein in the first ejector position the cartridge is not in the auto injector.

63. The auto injector according to claim 61, wherein ejector member is an ejector resilient member, and wherein when the cartridge is received within the auto injector, the ejector resilient member is compressed and the blocking member is moved to the blocking position.

64. The auto injector according to claim 61, wherein the auto injector further comprises an ejector lock configured for rotating at least a fraction of a revolution from an initial angular position to a first angular position when the plunger rod moves from the retracted plunger rod position towards the extended plunger rod position, wherein the rotation of the ejector lock retains the ejector member in a longitudinal and/or rotational position.

65. The auto injector according to claim 61, wherein the ejector member comprises an ejector support face that supports the cartridge and the cartridge holder if the cartridge is received in the cartridge receiver, wherein when the rotation of the ejector lock retains the ejector member in a longitudinal and/or rotational position, the cartridge and the cartridge holder are also retained in a longitudinal and/or rotational position.

66. The auto injector according to claim 61, wherein the blocking member remains in the blocking position during the auto injector process.

67. A system comprising an auto injector according to claim 1 and a cartridge comprising a cartridge outlet, a cartridge compartment containing the medicament, and a first stopper.

68. A method for ensuring that a re-chargeable battery in an auto injector contains sufficient voltage level to allow for the auto injector to perform an auto injector process,
wherein the auto injector comprises:
a cartridge receiver configured to receive a cartridge containing medicament;
a temperature sensor configured to measure a temperature of the auto injector;
a re-chargeable battery configured to power at least a drive module for moving a plunger rod in the auto injector;
a battery calculation module configured to calculate a residual electrical battery voltage level of the re-chargeable battery;
a processing unit coupled to the temperature sensor, the battery calculation module, and the drive module, the processing unit being configured to perform the method;
wherein the method comprises:
receiving the measured temperature from the temperature sensor;
receiving the calculated residual electrical battery voltage level of the re-chargeable battery from the battery calculation module;
obtaining a predefined threshold value indicative of the minimum electrical battery voltage level needed for performing the auto injector process at the measured temperature;
comparing the calculated residual electrical battery voltage level with the obtained predefined threshold value;
initiating the auto injector process only if the calculated residual electrical battery voltage level is larger than the predefined threshold value.

69. The method according to claim 68, further comprising instructing the user to recharge the battery if the calculated residual electrical battery voltage level is smaller than the predefined threshold value needed to perform the auto injector process at the measured temperature.
